(19) United States
(12) Patent Application Publication
(10) Pub. No.: US 2022/0177446 A1
(43) Pub. Date: Jun. 9, 2022

(54) ALKYNE COMPOUNDS AS IRAK INHIBITORS, PREPARATION METHODS AND MEDICINAL USES THEREOF

(71) Applicant: Eternity Bioscience Inc., Cranbury, NJ (US)

(72) Inventors: Hongbin Li, Newtown, CT (US); Bin Cao, Cranbury, NJ (US); Jian Liu, Edison, NJ (US); Linghang Zhuang, Chalfont, PA (US); Tongyuan Li, Fort Lee, NJ (US); Suxing Liu, Edison, NJ (US)

(21) Appl. No.: 17/540,907

(22) Filed: Dec. 2, 2021

Related U.S. Application Data
(60) Provisional application No. 63/120,966, filed on Dec. 3, 2020.

(51) Int. Cl. C07D 401/12; C07D 401/14

(52) U.S. Cl. CPC C07D 401/12; C07D 401/14

(57) ABSTRACT

This application discloses alkyne compounds of formula (I) useful as IRAK inhibitors and therapeutic agents for treatment of IRAK, especially IRAK-4, mediated disease or disorders, including autoimmune diseases, cancers, neurodegenerative disorders, viral diseases, and inflammatory disorders, hereditary disorders, and so on. The application also discloses pharmaceutical compositions containing these compounds, and synthetic methods and medical uses of the compounds.

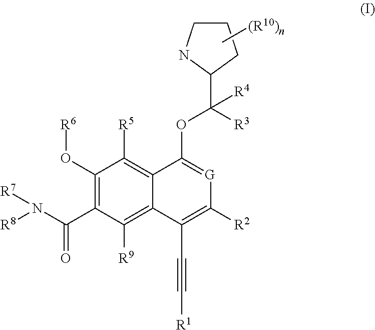

### CROSS-REFERENCE TO RELATED APPLICATIONS

This application claims priority under 35 U.S.C. § 119(e) to U.S. Provisional Patent Application No. 63/120,966, filed on Dec. 3, 2020, the disclosure of which is incorporated herein by reference in its entirety.

### FIELD OF THE DISCLOSURE

The present disclosure relates to compounds and methods useful in the inhibition of the function of interleukin-1 receptor-associated kinase (IRAK) and accordingly may have a beneficial impact on the therapies of IRAK mediated diseases or disorders.

### BACKGROUND OF THE DISCLOSURE

As a central signaling mediator, interleukin-1 receptor-associated kinase 4 (IRAK4) plays crucial roles in transducing the responses of the interleukin-1 (IL-1) receptors and Toll-like receptors (TLR). IRAK4 overactivation is linked with various types of human autoimmune and inflammatory diseases. A number of studies have reported that all TLR/IL-1Rs with known function are involved in host defense mechanisms, either by the recognition of pathogens or as receptors for inflammatory cytokines. They play a vital role in both innate and adaptive immunity in mammals, and the signaling cascades mediated by these receptors are associated with various human diseases. IRAK proteins are key components in the signal transduction pathways mediated by interleukin-1 receptor (IL-IR), interleukin-18 receptor (IL-18R), and Toll-like receptors (TLRs).

IRAK family consists of four members, including IRAK-1, IRAK-2, IRAK-M, also denoted IRAK-3, and IRAK-4. Among them, IRAK1 and IRAK4 proteins are catalytically active kinases, whereas IRAK2 and IRAK3 are inactive pseudokinases (Jain et al., Front. Immunol., 2014, 5(553): 1-8). IRAK-4 belongs to the serine/threonine kinases and is the best characterized member of the IRAK family. IRAK-4 is involved in signaling innate immune responses from both TLRs and IL-1 receptors. Innate immunity detects pathogens through the recognition of pathogen-associated molecular patterns (PAMPs) by TLRs. TLRs recognize conserved structures of both microbes and endogenous molecules. The cell surface TLRs recognize bacterial and fungal components, whereas TLRs recognizing viral or microbial nucleic acids are localized into intracellular membranes such as endosomes and phagosomes. Upon ligand binding, these receptors recruit the scaffolding adaptor protein myeloid differentiation primary response gene (88) (MyD88) through the Toll/interleukin-1 (IL-1) receptor (TTR) domain. MyD88 is the universal adaptor used by all TLRs except for TLR3. MyD88 couples to pathways that lead to the activation of transcription factors such as NF-κB (nuclear factor-KB), IRF1 (IFN-regulatory factor 1), IRF5 and IRF7 (Luke A. J. O'Neill & Andrew G. Bowie, Nat. Rev. Immunol., 2007, 7: 353-364). MyD88 utilizes the death domain to recruit IRAK4, thereby activating its kinase function to phosphorylate IRAK1 and IRAK2 within the myddosome complex. Phosphorylation at multiple sites allows IRAK to dissociate from the myddosome complex and activate the downstream proteins such as TNF receptor-associated factor 6 (TRAF-6), which finally can initiate downstream activation of NF-kB and MAPK signaling pathways, leading to the induction of pro-inflammatory cytokines and chemokines, such as TNF-a, IL-6, and IL-8.

Mice homozygous for disruptions in this gene display an essentially normal phenotype, and in overexpression studies, IRAK-2 and IRAK-3 can compensate for IRAK-1 loss in a mutant 293 cell line. Furthermore, in vitro assay demonstrated that kinase activity of IRAK1 is not required for IL-1 downstream signaling (Knop and Martin, FEBS Letters, 1999, 448: 81-85; Thomas, J A. et al., J. Immunol., 1999, 163:978-984; Wesche, H. et al., J. Biol. Chem., 1999, 274:19403-19410; Aravind L., et al., Science, 2001, 291:1279-1284). Both IRAK-4 and MyD88 deficient humans show normal resistance to common fungi, parasites, viruses, and many bacteria, except the susceptibility to bacterial infections, particularly recurrent pyogenic bacterial infections such as Streptococcus infections (Picard, C. al., Science, 2003 299:2076-2079; Ku, C-L. et al., J. Exp. Med., 2007, 204(10):2407-2022). Human germline gain-of-function MyD88 mutation which causes the constitutive activation of IRAK4 signaling led to severe arthritis (Sikora et al., J. Allergy Clin. Immunol., 2018, 141(5):1943-1947; Picard, C, et al., Clin. Microbiol. Rev., 2011, 24(3): 490-497; Koziczak-Holbro et al., Arthritis Rheum., 2009, 60(6): 1661-1671). IRAK1-deficient human peripheral blood mononuclear cells (PBMCs) have normal responses to both TLR agonists and IL-1β, but the responses in IRAK4 deficient human PBMCs are totally abolished (Mina et al., PNAS, 2017, E514-E523). Mouse knock-out experiments have demonstrated an essential role for IRAK-4 in IL-IR, IL-18R and most TLR signaling. IRAK4-deficient mice also exhibit defective innate immunity and are more susceptible to bacterial infection. IRAK4 kinase dead mice are protected against antigen-induced arthritis. (Suzuki, N. et al., Nature, 2002, 416:750-756).

IRAK4 also plays a central role in tumor growth and progression. Inhibition of IRAK4 is universally toxic towards activated B-cell diffuse large B-cell lymphoma (ABC DLBCL) but not germinal center B-cell (GCB) DLBCL cell lines, mechanistic studies demonstrated that IRAK4 inhibition potently abrogates IRAK4-mediated phosphorylation of IRAK1 and the downstream activation of NF-1B and MAPK signaling pathways resulting from the MyD88 (L265P) mutation in ABC DLBCL, leading to the suppression of secretion of the pro-inflammatory cytokines TNFα, IL-6 and IL-10 by ABC DLBCL cells. Furthermore, aberrant B cell receptor (BCR) signaling in ABC DLBCL is also engaged in tumor growth and progression, and this chronic active BCR signaling can be blocked by inhibiting Bruton's tyrosine kinase (BTK) pharmacologically. Notably, the IRAK4 inhibitors strongly synergized with BTK inhibition in killing multiple ABC DLBCL cell lines. (Ngo et al., Nature, 2011, 470(7332): 115-119; Lim et al., Blood, 2012, 120(21):625).

Toll/IL-1 receptor family members like IRAK4 are central components of host defense mechanisms in a variety of species and IRAK4-mediated signaling, downstream of TLRs and the IL-1 receptor family, bridges both innate and adaptive immunity. Both TLRs and IL-1 pathways have been linked to immune and inflammatory diseases such as chronic inflammatory diseases, including chronic arthritis, atherosclerosis, multiple sclerosis, cancers, and autoimmune disorders including rheumatoid arthritis, lupus, asthma, psoriasis, and inflammatory bowel diseases. Overall, IRAK4 plays a key role in immune and inflammatory responses and is considered as an important potential therapeutic target for autoimmune diseases, inflammatory diseases, and cancer.

Patent applications for compounds that have been published as IRAK4 inhibitors include WO2015150995A1, WO2019089422A1, WO2020113233A1, WO2019133531A1, WO2015048281A1, WO2017025849A1, WO2017033093A1, WO2018209012A1 and WO2019149522A1. However, there is still a need to develop new IRAK, especially IRAK4, inhibitors to meet the unmet medical needs.

### SUMMARY OF THE DISCLOSURE

The compounds of this disclosure inhibit the function of IRAK and accordingly may serve as therapeutic agents for the treatment of diseases including a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

The present disclosure, in one aspect, provides a compound of formula (I), or a tautomer, mesomer, racemate, enantiomer, diastereomer thereof, or mixture thereof, or a pharmaceutically acceptable salt thereof:

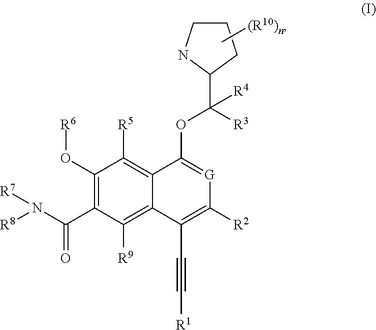

wherein:

G is N or CR^{g};

R¹ is selected from C₄₋₆ alkyl, alkyl substituted by R (i.e., R-alkyl), —CONR^{m}Rⁿ, —COOR^{p}, aryl and heteroaryl, wherein the aryl or heteroaryl is optionally substituted with one or more, preferably one to five, and sometimes more preferably one to three, groups independently selected from hydrogen, halogen, alkyl, haloalkyl, alkoxy, haloalkoxy, hydroxyl, hydroxyalkyl, cyano, amino, nitro, —(CH₂)ᵣ—NR^{a}R^{b}, —C(═O)R^{c}, —OC(═O)R^{c}, —OC(═O)OR^{a}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl;

R is selected from halogen, alkyl, haloalkyl, alkoxy, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl;

R², R⁵, R⁹ and R^{g} are identical or different and are independently selected from hydrogen, halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b}, nitro, cycloalkyl, heterocyclyl, aryl and heteroaryl; wherein the alkyl, cycloalkyl, heterocyclyl, aryl or heteroaryl is optionally substituted with one or more, preferably one to five, and sometimes more preferably one to three, groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;

R³ and R⁴ are identical or different and are independently selected from hydrogen, halogen, alkyl, haloalkyl, alkoxy, alkylthio, haloalkoxy, haloalkylthio, hydroxyl, hydroxyalkyl, cyano, amino and —(CH₂)ᵣ—NR^{a}R^{b};

R⁶ is selected from alkyl, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more, preferably one to five, and sometimes more preferably one to three, groups independently selected from halogen, alkyl, haloalkyl, alkoxy, alkylthio, haloalkoxy, hydroxyl, hydroxyalkyl, cyano, amino, nitro, —(CH₂)ₛ—NR^{e}R^{f} and cycloalkyl;

R¹⁰ at each occurrence is independently selected from hydrogen, halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b}, nitro, oxo, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more, preferably one to five, and sometimes more preferably one to three, groups independently selected from halogen, alkyl, haloalkyl, alkoxy, alkylthio, haloalkoxy, haloalkylthio, hydroxyl, hydroxyalkyl, cyano, amino, nitro, —(CH₂)ₛ—NR^{e}R^{f} and cycloalkyl;

or two R¹⁰ together with the attached atoms form a cycloalkyl or heterocyclyl; wherein the cycloalkyl and heterocyclyl are optionally substituted with one or more, preferably one to five, and sometimes more preferably one to three, groups independently selected from halogen, alkyl, haloalkyl, alkoxy, alkylthio, haloalkoxy, haloalkylthio, hydroxyl, hydroxyalkyl, cyano, amino, nitro and —(CH₂)ₛ—NR^{c}R^{f};

R⁷, R⁸, R^{a}, R^{b}, R^{e}, R^{f}, R^{p}, R^{m} and Rⁿ are identical or different and at each occurrence are independently selected from hydrogen, alkyl, haloalkyl, hydroxyalkyl, —C(═O)OR^{q}, cycloalkyl, heterocyclyl, aryl and heteroaryl;

or R⁷ and R⁸, R^{a} and R^{b}, R^{e} and R^{f}, R^{m} and Rₙ together with the nitrogen to which they are attached form a heterocyclyl; wherein the heterocyclyl is optionally substituted with one or more, preferably one to five, and sometimes more preferably one to three, groups independently selected from halogen, alkyl, haloalkyl, alkoxy, haloalkoxy, hydroxyl, hydroxyalkyl, oxo, cyano, amino, nitro, cycloalkyl, heterocyclyl, aryl and heteroaryl;

R^{c} at each occurrence is independently selected from alkyl, cycloalkyl, heterocyclyl, aryl and heteroaryl, wherein the alkyl, cycloalkyl, heterocyclyl, aryl or heteroaryl is optionally substituted with one or more, preferably one to five, and sometimes more preferably one to three, groups independently selected from halogen, alkyl, haloalkyl, alkoxy, haloalkoxy, hydroxyl, hydroxyalkyl, cyano, amino and nitro;

R^{d} at each occurrence is independently selected from hydrogen, alkyl, haloalkyl, hydroxyalkyl and cycloalkyl, wherein the cycloalkyl is optionally substituted with one or more, preferably one to five, and sometimes more preferably one to three, groups independently selected from halogen, alkyl, haloalkyl, alkoxy, haloalkoxy, hydroxyl, hydroxyalkyl, cyano, amino, and nitro;

R^{q} is selected from hydrogen, alkyl and haloalkyl;

n is 0, 1, 2, 3 or 4;

r is 0, 1, 2 or 3; and

s is 0, 1, 2 or 3.

In another aspect, this disclosure provides a preparation process of a compound of formula (I), or a tautomer, mesomer, racemate, enantiomer, diastereomer, or mixture thereof, or a pharmaceutically acceptable salt thereof, the preparation process comprising the steps of:

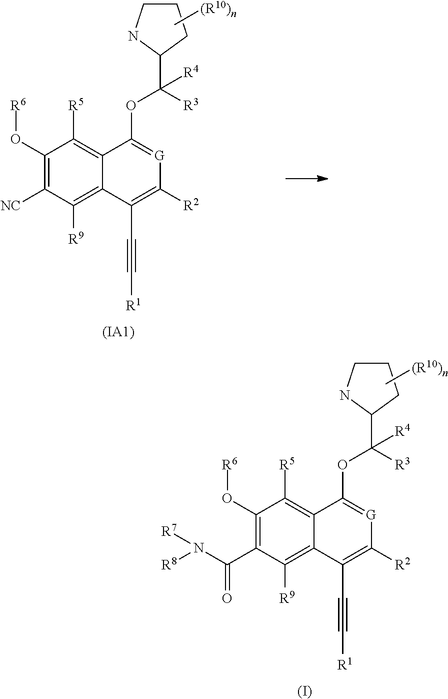

the compound of formula (IA1) is subjected to hydration reaction to obtain the compound of formula (I), wherein:

R⁷ is hydrogen;

R⁸ is hydrogen;

G, R¹ to R⁶, R⁹, R¹⁰ and n are each as defined in formula (I).

In another aspect, the present disclosure provides a pharmaceutical composition comprising a compound of formula (I), or a tautomer, mesomer, racemate, enantiomer, diastereomer, or mixture thereof, or a pharmaceutically acceptable salt thereof, and a pharmaceutically acceptable carrier.

In another aspect, the present disclosure provides a method of preventing and/or treating a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder, comprising a step of administering to a subject in need thereof a therapeutically effective amount of a compound of formula (I), or an isomer, pharmaceutically acceptable salt, or a pharmaceutical composition containing the compound.

In another aspect, the present disclosure also relates to use of a compound of formula (I), or an isomer, pharmaceutically acceptable salt thereof, or a pharmaceutical composition containing the compound, in the manufacture of a medicament for treatment of a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

Other aspects or advantages of the disclosure will be better appreciated in view of the following detailed description, examples, and claims.

### DETAILED DESCRIPTION OF THE DISCLOSURE

In one aspect, the present disclosure provides a compound of formula (I):

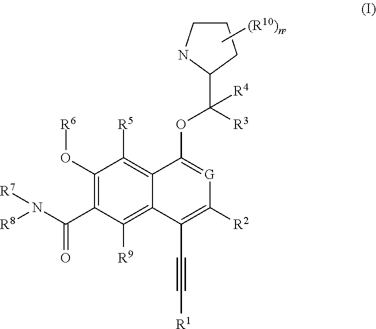

or a tautomer, mesomer, racemate, enantiomer, diastereomer, or mixture thereof, or pharmaceutically acceptable salt thereof, wherein:

G is N or CR^{g};

R¹ is selected from C₄₋₆ alkyl, alkyl substituted by R (i.e., R-alkyl), —CONR^{m}Rⁿ, —COOR^{p}, aryl and heteroaryl, wherein the aryl or heteroaryl is optionally substituted with one or more, preferably one to five, and sometimes more preferably one to three, groups independently selected from hydrogen, halogen, alkyl, haloalkyl, alkoxy, haloalkoxy, hydroxyl, hydroxyalkyl, cyano, amino, nitro, —(CH₂)ᵣ—NR^{a}R^{b}, —C(═O)R^{c}, —OC(═O)R^{c}, —OC(═O)OR^{a}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl;

R is selected from halogen, alkyl, haloalkyl, alkoxy, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl;

R², R⁵, R⁹ and R^{g} are identical or different and are independently selected from hydrogen, halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b}, nitro, cycloalkyl, heterocyclyl, aryl and heteroaryl; wherein the alkyl, cycloalkyl, heterocyclyl, aryl or heteroaryl is optionally substituted with one or more, preferably one to five, and sometimes more preferably one to three, groups independently selected from halogen, alkyl, haloalkyl, alkoxy, haloalkoxy, hydroxyl, hydroxyalkyl, cyano, amino, nitro, —(CH₂)ₛ—NR^{c}R^{f}, cycloalkyl, heterocyclyl, aryl and heteroaryl;

R³ and R⁴ are identical or different and are independently selected from hydrogen, halogen, alkyl, haloalkyl, alkoxy, alkylthio, haloalkoxy, haloalkylthio, hydroxyl, hydroxyalkyl, cyano, amino and —(CH₂)ᵣ—NR^{a}R^{b};

R⁶ is selected from alkyl, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more, preferably one to five, and sometimes more preferably one to three, groups independently selected from halogen, alkyl, haloalkyl, alkoxy, alkylthio, haloalkoxy, hydroxyl, hydroxyalkyl, cyano, amino, nitro, —(CH₂)ₛ—NR^{e}R^{f} and cycloalkyl;

R¹⁰ at each occurrence is independently selected from hydrogen, halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b}, nitro, oxo, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more, preferably one to five, and sometimes more preferably one to three, groups independently selected from halogen, alkyl, haloalkyl, alkoxy, alkylthio, haloalkoxy, haloalkylthio, hydroxyl, hydroxyalkyl, cyano, amino, nitro, —(CH₂)ₛ—NR^{e}R^{f} and cycloalkyl;

or two R¹⁰ together with the attached atoms form a cycloalkyl or heterocyclyl; wherein the cycloalkyl and heterocyclyl are optionally substituted with one or more, preferably one to five, and sometimes more preferably one to three, groups independently selected from halogen, alkyl, haloalkyl, alkoxy, alkylthio, haloalkoxy, haloalkylthio, hydroxyl, hydroxyalkyl, cyano, amino, nitro and —(CH₂)ₛ—NR^{e}R^{f};

R⁷, R⁸, R^{a}, R^{b}, R^{e}, R^{f}, R^{p}, R^{m} and Rⁿ are identical or different and at each occurrence are independently selected from hydrogen, alkyl, haloalkyl, hydroxyalkyl, —C(═O)OR^{q}, cycloalkyl, heterocyclyl, aryl and heteroaryl;

or R⁷ and R⁸, R^{a} and R^{b}, R^{e} and R^{f}, R^{m} and Rⁿ together with the nitrogen to which they are attached form a heterocyclyl; wherein the heterocyclyl is optionally substituted with one or more, preferably one to five, and sometimes more preferably one to three, groups independently selected from halogen, alkyl, haloalkyl, alkoxy, haloalkoxy, hydroxyl, hydroxyalkyl, oxo, cyano, amino, nitro, cycloalkyl, heterocyclyl, aryl and heteroaryl;

R^{c} at each occurrence is independently selected from alkyl, cycloalkyl, heterocyclyl, aryl and heteroaryl, wherein the alkyl, cycloalkyl, heterocyclyl, aryl or heteroaryl is optionally substituted with one or more, preferably one to five, and sometimes more preferably one to three, groups independently selected from halogen, alkyl, haloalkyl, alkoxy, haloalkoxy, hydroxyl, hydroxyalkyl, cyano, amino and nitro;

R^{d} at each occurrence is independently selected from hydrogen, alkyl, haloalkyl, hydroxyalkyl and cycloalkyl, wherein the cycloalkyl is optionally substituted with one or more, preferably one to five, and sometimes more preferably one to three, groups independently selected from halogen, alkyl, haloalkyl, alkoxy, haloalkoxy, hydroxyl, hydroxyalkyl, cyano, amino, and nitro;

R^{g} is selected from hydrogen, alkyl and haloalkyl;

n is 0, 1, 2, 3 or 4;

r is 0, 1, 2 or 3; and

s is 0, 1, 2 or 3.

In some embodiments of the disclosure, in the compound of formula (I), or a tautomer, mesomer, racemate, enantiomer, diastereomer, or mixture thereof, or a pharmaceutically acceptable salt thereof, R¹ is aryl or heteroaryl; wherein the aryl or heteroaryl is optionally substituted with one or more, preferably one to five, and sometimes more preferably one to three, groups independently selected from halogen, alkyl, haloalkyl, alkoxy, haloalkoxy, hydroxyl, hydroxyalkyl, cyano, amino and nitro.

In some embodiments, preferably R¹ is 6 membered aryl or 5-6 membered heteroaryl; wherein the 6 membered aryl or 5-6 membered heteroaryl is optionally substituted with one or more, preferably one to five, and sometimes more preferably one to three, groups independently selected from halogen, C₁₋₆ alkyl, C₁₋₆ haloalkyl, C₁₋₆ alkoxy, C₁₋₆ haloalkoxy, hydroxyl, C₁₋₆ hydroxyalkyl, cyano and amino.

In some embodiments, more preferably R¹ is 6 membered aryl which is optionally substituted with one or more, preferably one to five, and sometimes more preferably one to three, groups independently selected from halogen, C₁₋₆ alkyl, C₁₋₆ haloalkyl, C₁₋₆ alkoxy, C₁₋₆ haloalkoxy and cyano.

In some embodiments of the disclosure, in the compound of formula (I), or a tautomer, mesomer, racemate, enantiomer, diastereomer, or mixture thereof, or a pharmaceutically acceptable salt thereof, R¹ is selected from alkyl substituted by R, —CONR^{m}Rⁿ and —COOR^{p}; R is selected from halogen, alkyl, haloalkyl, alkoxy, haloalkoxy, hydroxyl, hydroxyalkyl, cyano, amino, nitro, —(CH₂)ᵣ—NR^{a}R^{b}, cycloalkyl, heterocyclyl, aryl and heteroaryl; wherein R^{a}, R^{b}, R^{p}, R^{m}, Rⁿ and r are defined in formula (I).

In some embodiments, preferably R¹ is C₁₋₆ alkyl substituted by R; R is selected from halogen, C₁₋₆ alkoxy, C₁₋₆ haloalkoxy, hydroxyl, cyano, amino and —NR^{a}R^{b}; wherein R^{a} and R^{b} are C₁₋₆ alkyl.

In some embodiments of the disclosure, in the compound of formula (I), or a tautomer, mesomer, racemate, enantiomer, diastereomer, or mixture thereof, or a pharmaceutically acceptable salt thereof, R¹ is selected from —CONR^{m}Rⁿ and —COOR^{p}; wherein R^{m}, Rⁿ and R^{p} are identical or different and at each occurrence are independently selected from hydrogen, C₁₋₆alkyl, C₁₋₆haloalkyl, C₁₋₆hydroxyalkyl and 3-6 membered cycloalkyl.

In some embodiments, more preferably R¹ is selected from —CONR^{m}Rⁿ and —COOR^{p}; wherein R^{m} and Rⁿ are C₁₋₆ alkyl; R^{P} is hydrogen.

In some embodiments of the disclosure, the compound of formula (I), or a tautomer, mesomer, racemate, enantiomer, diastereomer, or mixture thereof, or a pharmaceutically acceptable salt thereof, is a compound of formula (II), or a tautomer, mesomer, racemate, enantiomer, diastereomer, or mixture thereof, or a pharmaceutically acceptable salt thereof:

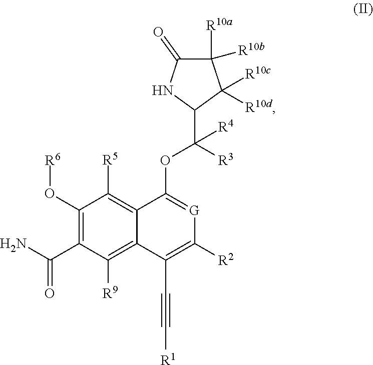

wherein R^{10a}, R^{10b}, R^{10c} and R^{10d} are identical or different and at each occurrence are independently selected from hydrogen, halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b} and cycloalkyl; G, R¹-R⁶ and R⁹ are each as defined in formula (I).

In some embodiments, preferably R^{10a} and R^{10c} are hydrogen, R^{10b} is fluoro, and R^{10d} is ethyl.

In some embodiments of the disclosure, the compound of formula (I), or a tautomer, mesomer, racemate, enantiomer, diastereomer, or mixture thereof, or a pharmaceutically acceptable salt thereof, is a compound of formula (III), or a tautomer, mesomer, racemate, enantiomer, diastereomer, or mixture thereof, or a pharmaceutically acceptable salt thereof:

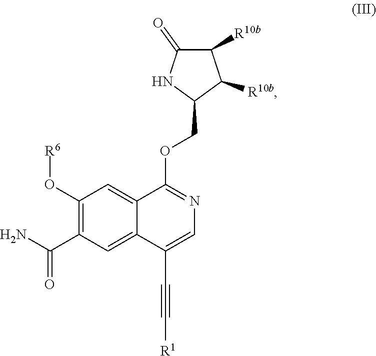

wherein:

R^{10b} and R^{10d} are identical or different and at each occurrence are independently selected from halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b} and cycloalkyl;

R¹, R⁶, R^{a}, R^{b} and r are each as defined in formula (I) above.

In some embodiments of the disclosure, in the compound of formula (I), or a tautomer, mesomer, racemate, enantiomer, diastereomer, or mixture thereof, or a pharmaceutically acceptable salt thereof, R² is selected from hydrogen, halogen, alkyl, haloalkyl, alkoxy, alkylthio, haloalkoxy, haloalkylthio, hydroxyl, hydroxyalkyl, cyano, amino, —NR^{a}R^{b}, nitro and cycloalkyl; wherein R^{a} and R^{b} are alkyl.

In some embodiments, preferably R² is hydrogen, halogen or C₁₋₆ alkyl; and more preferably hydrogen.

In some embodiments of the disclosure, in the compound of formula (I), or a tautomer, mesomer, racemate, enantiomer, diastereomer, or mixture thereof, or a pharmaceutically acceptable salt thereof, R³ is selected from hydrogen, halogen, alkyl, haloalkyl, hydroxyl, hydroxyalkyl, cyano and amino.

In some embodiments, preferably R³ is hydrogen or C₁₋₆ alkyl; and more preferably hydrogen.

In some embodiments of the disclosure, in the compound of formula (I), or a tautomer, mesomer, racemate, enantiomer, diastereomer, or mixture thereof, or a pharmaceutically acceptable salt thereof, R⁴ is selected from hydrogen, halogen, alkyl, haloalkyl, hydroxyl, hydroxyalkyl, cyano and amino.

In some embodiments, preferably R⁴ is hydrogen or C₁₋₆ alkyl; and more preferably hydrogen.

In some embodiments of the disclosure, in the compound of formula (I), or a tautomer, mesomer, racemate, enantiomer, diastereomer, or mixture thereof, or a pharmaceutically acceptable salt thereof, R⁵ is selected from hydrogen, halogen, alkyl, haloalkyl, alkoxy, alkylthio, haloalkoxy, haloalkylthio, hydroxyl, hydroxyalkyl, cyano, amino, —NR^{a}R^{b}, nitro and cycloalkyl; wherein R^{a} and R^{b} are alkyl.

In some embodiments, preferably R⁵ is hydrogen, halogen or C₁₋₆ alkyl; and more preferably hydrogen.

In some embodiments of the disclosure, in the compound of formula (I), or a tautomer, mesomer, racemate, enantiomer, diastereomer, or mixture thereof, or a pharmaceutically acceptable salt thereof, R⁹ is selected from hydrogen, halogen, alkyl, haloalkyl, alkoxy, alkylthio, haloalkoxy, haloalkylthio, hydroxyl, hydroxyalkyl, cyano, amino, —NR^{a}R^{b}, nitro and cycloalkyl; wherein R^{a} and R^{b} are alkyl.

In some embodiments, preferably R⁹ is hydrogen, halogen or C₁₋₆ alkyl; and more preferably hydrogen.

In some embodiments of the disclosure, in the compound of formula (I), or a tautomer, mesomer, racemate, enantiomer, diastereomer, or mixture thereof, or a pharmaceutically acceptable salt thereof, R⁶ is selected from alkyl, alkenyl, alkynyl and cycloalkyl.

In some embodiments, preferably R⁶ is C₁₋₆ alkyl; and more preferably methyl or isopropyl.

In some embodiments of the disclosure, in the compound of formula (I), or a tautomer, mesomer, racemate, enantiomer, diastereomer, or mixture thereof, or a pharmaceutically acceptable salt thereof, R⁷ is hydrogen, haloalkyl or C₁₋₆ alkyl.

In some embodiments, preferably R⁷ is hydrogen.

In some embodiments of the disclosure, in the compound of formula (I), or a tautomer, mesomer, racemate, enantiomer, diastereomer, or mixture thereof, or a pharmaceutically acceptable salt thereof, R⁷ is hydrogen, haloalkyl or C₁₋₆ alkyl.

In some embodiments, preferably R⁷ is hydrogen.

In some embodiments of the disclosure, in the compound of formula (I), or a tautomer, mesomer, racemate, enantiomer, diastereomer, or mixture thereof, or a pharmaceutically acceptable salt thereof, R¹⁰ is selected from hydrogen, halogen, alkyl, haloalkyl, alkoxy, haloalkoxy, hydroxyl, hydroxyalkyl, cyano, amino, —NR^{a}R^{b}, oxo and cycloalkyl; wherein R^{a} and R^{b} are alkyl.

In some embodiments, preferably R¹⁰ is selected from hydrogen, halogen, C₁₋₆ alkyl and oxo.

In some embodiments, more preferably R¹⁰ is fluoro or ethyl.

In some embodiments of the disclosure, in the compound of formula (I), or a tautomer, mesomer, racemate, enantiomer, diastereomer, or mixture thereof, or a pharmaceutically acceptable salt thereof, R^{10a} and R^{10b} are identical or different and at each occurrence are independently selected from hydrogen, halogen, alkyl, haloalkyl, alkoxy, haloalkoxy, hydroxyl, hydroxyalkyl, cyano, amino, —NR^{a}R^{b}; wherein R^{a} and R^{b} are alkyl.

In some embodiments, preferably R^{10a} and R^{10b} are independently selected from hydrogen, halogen and C₁₋₆ alkyl.

In some embodiments, more preferably R^{10a} and R^{10b} are independently hydrogen or fluoro.

In some embodiments of the disclosure, in the compound of formula (I), or a tautomer, mesomer, racemate, enantiomer, diastereomer, or mixture thereof, or a pharmaceutically acceptable salt thereof, R^{10c} and R^{10d} are identical or different and at each occurrence are independently selected from hydrogen, halogen, alkyl, haloalkyl, alkoxy, haloalkoxy, hydroxyl, hydroxyalkyl, cyano, amino, —NR^{a}R^{b}; wherein R^{a} and R^{b} are alkyl.

In some embodiments, preferably R^{10c} and R^{10d} are independently selected from hydrogen, halogen and C₁₋₆ alkyl.

In some embodiments, more preferably R^{10c} and R^{10d} are independently hydrogen or ethyl.

In some embodiments of the disclosure, in the compound of formula (I), or a tautomer, mesomer, racemate, enantiomer, diastereomer, or mixture thereof, or a pharmaceutically acceptable salt thereof, R^{g} is selected from hydrogen, halogen, alkyl, haloalkyl, alkoxy, alkylthio, haloalkoxy, haloalkylthio, hydroxyl, hydroxyalkyl, cyano, amino, —NR^{a}R^{b}, nitro and cycloalkyl; wherein R^{a} and R^{b} are alkyl.

In some embodiments, preferably R^{g} is hydrogen, halogen or C₁₋₆ alkyl.

In some embodiments, more preferably R^{g} is hydrogen.

In some embodiments of the disclosure, in the compound of formula (I), or a tautomer, mesomer, racemate, enantiomer, diastereomer, or mixture thereof, or a pharmaceutically acceptable salt thereof, R^{a}, R^{b}, R^{e}, R^{f}, R^{p}, R^{m} and Rⁿ are identical or different and at each occurrence are independently selected from hydrogen, C₁₋₆ alkyl, C₁₋₆ haloalkyl and 3-6 membered cycloalkyl.

In some embodiments, preferably R^{a}, R^{b}, R^{e}, R^{f}, R^{p}, R^{m} and Rⁿ are independently hydrogen or C₁₋₆ alkyl.

In some embodiments of the disclosure, in the compound of formula (I), or a tautomer, mesomer, racemate, enantiomer, diastereomer, or mixture thereof, or a pharmaceutically acceptable salt thereof, R^{q} is hydrogen or C₁₋₆ alkyl.

In some embodiments of the disclosure, in the compound of formula (I), or a tautomer, mesomer, racemate, enantiomer, diastereomer, or mixture thereof, or a pharmaceutically acceptable salt thereof, n is 1, 2 or 3.

In some embodiments, preferably n is 3.

In some embodiments of the disclosure, in the compound of formula (I), or a tautomer, mesomer, racemate, enantiomer, diastereomer, or mixture thereof, or a pharmaceutically acceptable salt thereof, r is 0, 1 or 2.

In some embodiments, preferably r is 0 or 1.

In some embodiments of the disclosure, in the compound of formula (I), or a tautomer, mesomer, racemate, enantiomer, diastereomer, or mixture thereof, or a pharmaceutically acceptable salt thereof, s is 0, 1 or 2.

In some embodiments, preferably s is 0 or 1.

As a person of ordinary skill in the art would understand, any and all plausible combinations of the embodiments disclosed herein, in particular, with regard to the definitions of any substituents, e.g., G, R¹ to R¹⁰, and n, or the like, in the compounds of formula (I), or the like, are all encompassed by the present disclosure.

For example, in one embodiment, the present disclosure provides a compound of formula (I), or a tautomer, mesomer, racemate, enantiomer, diastereomer, or mixture thereof, or a pharmaceutically acceptable salt thereof, wherein:

R¹ is C₆₋₁₀ aryl or 5- to 10-membered heteroaryl, wherein the aryl or heteroaryl is optionally substituted with one or more groups independently selected from halogen, alkyl, haloalkyl, alkoxy, haloalkoxy, hydroxyl, hydroxyalkyl, cyano, amino and nitro;

R² is hydrogen, halogen or C₁₋₆ alkyl;

R³ is hydrogen or C₁₋₆ alkyl;

R⁴ is hydrogen or C₁₋₆ alkyl;

R⁵ is hydrogen, halogen or C₁₋₆ alkyl;

R⁶ is C₁₋₆ alkyl;

R⁷ is hydrogen or C₁₋₆ alkyl;

R⁸ is hydrogen or C₁₋₆ alkyl;

R⁹ is hydrogen, halogen or C₁₋₆ alkyl; and

R¹⁰ is selected from hydrogen, halogen, C₁₋₆ alkyl and oxo.

In one embodiment, the present disclosure provides a compound of formula (I), or a tautomer, mesomer, racemate, enantiomer, diastereomer, or mixture thereof, or a pharmaceutically acceptable salt thereof, wherein:

R¹ is selected from —CONR^{m}Rⁿ and —COOR^{p}, and C₁₋₆ alkyl substituted by R;

R² is hydrogen, halogen or C₁₋₆ alkyl;

R³ is hydrogen or C₁₋₆ alkyl;

R⁴ is hydrogen or C₁₋₆ alkyl;

R⁵ is hydrogen, halogen or C₁₋₆ alkyl;

R⁶ is C₁₋₆ alkyl; preferably methyl;

R⁷ is hydrogen or C₁₋₆ alkyl;

R⁸ is hydrogen or C₁₋₆ alkyl;

R⁹ is hydrogen, halogen or C₁₋₆ alkyl;

R¹⁰ is selected from hydrogen, halogen, C₁₋₆ alkyl and oxo;

R is selected from halogen, C₁₋₄ alkyl, C₁₋₄ haloalkyl, C₁₋₆ alkoxy, C₁₋₆ haloalkoxy, hydroxyl, hydroxyC₁₋₆alkyl, cyano, amino, nitro, —(CH₂)ᵣ—NR^{a}R^{b}, C₃₋₆ cycloalkyl, 5- to 10-membered heterocyclyl, C₆₋₁₀ aryl and 5- to 10-membered heteroaryl;

R^{p} is each independently selected from hydrogen, C₁₋₆ alkyl, C₁₋₆ haloalkyl, hydroxyC₁₋₆alkyl, C₃₋₆ cycloalkyl, 5- to 10-membered heterocyclyl, C₆₋₁₀ aryl and 5- to 10-membered heteroaryl;

R^{a}, R^{b}, R^{m} and Rⁿ are each independently selected from hydrogen, C₁₋₆ alkyl, and —C(═O)OR^{q},

or alternatively R^{a} and R^{b} or R^{m} and Rⁿ together with the nitrogen to which they are attached form a heterocyclyl, wherein the heterocyclyl is optionally substituted with one to three groups independently selected from halogen, C₁₋₆ alkyl, C₁₋₆ haloalkyl, C₁₋₆ alkoxy, C₁₋₆ haloalkoxy, hydroxyl, hydroxyC₁₋₆alkyl, oxo, cyano, amino, nitro, C₃₋₆ cycloalkyl, 5- to 10-membered heterocyclyl, C₆₋₁₀ aryl and 5- to 10-membered heteroaryl;

R^{q} is selected from hydrogen, C₁₋₆ alkyl and C₁₋₆ haloalkyl; and

r is 0, 1, 2 or 3.

Exemplified compounds of the disclosure include, but are not limited to:

| Example | |
|---|---|
| No. | Compound structure and name |
| 1 | 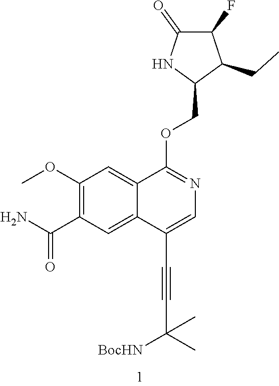 [SMILES: CC[C@@H]1[C@H](F)C(=O)N[C@@H]1COc1ncc(C#CC(C)(C)NC(=O)OC(C)(C)C)c2cc(C(N)=O)c(OC)cc12] |
| | Example 1: tert-butyl (4-(6-carbamoyl-1-(((2S,3S,4S)-3-ethyl-4-fluoro- |
| | 5-oxopyrrolidin-2-yl)methoxy)-7-methoxyisoquinolin-4-yl)-2- |
| | methylbut-3-yn-2-yl)carbamate 1 |
| 2 | 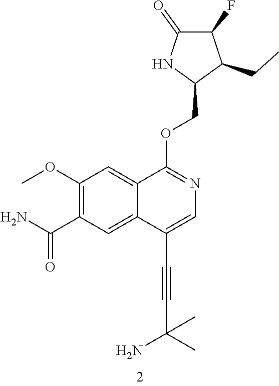 [SMILES: CC[C@@H]1[C@H](F)C(=O)N[C@@H]1COc1ncc(C#CC(C)(C)N)c2cc(C(N)=O)c(OC)cc12] |
| | Example 2: 4-(3-amino-3-methylbut-1-yn-1-yl)-1-(((2S,3S,4S)-3-ethyl- |
| | 4-fluoro-5-oxopyrrolidin-2-yl)methoxy)-7-methoxyisoquinoline-6- |
| | carboxamide 2 |
| 3 | 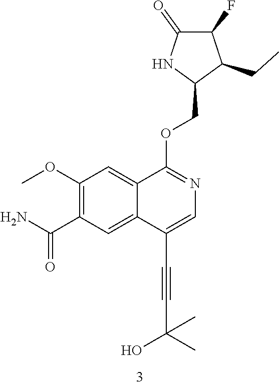 [SMILES: CC[C@@H]1[C@H](F)C(=O)N[C@@H]1COc1ncc(C#CC(C)(C)O)c2cc(C(N)=O)c(OC)cc12] |
| | Example 3: 1-(((2S,3S,4S)-3-ethyl-4-fluoro-5-oxopyrrolidin-2- |
| | yl)methoxy)-4-(3-hydroxyl-3-methylbut-1-yn-1-yl)-7- |
| | methoxyisoquinoline-6-carboxamide 3 |
| 4 | 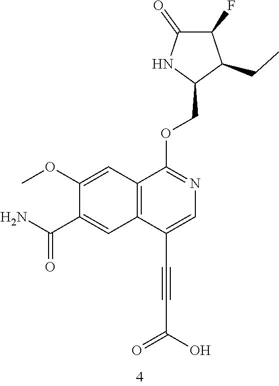 [SMILES: CC[C@@H]1[C@H](F)C(=O)N[C@@H]1COc1ncc(C#CC(=O)O)c2cc(C(N)=O)c(OC)cc12] |
| | Example 4: 3-(6-carbamoyl-1-(((2S,3S,4S)-3-ethyl-4-fluoro-5- |
| | oxopyrrolidin-2-yl)methoxy)-7-methoxyisoquinolin-4-yl)propiolic acid 4 |
| 5 | 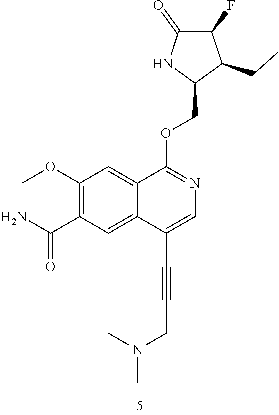 [SMILES: CC[C@@H]1[C@H](F)C(=O)N[C@@H]1COc1ncc(C#CCN(C)C)c2cc(C(N)=O)c(OC)cc12] |
| | Example 5: 4-(3-(dimethylamino)prop-1-yn-1-yl)-1-(((2S,3S,4S)-3- |
| | ethyl-4-fluoro-5-oxopyrrolidin-2-yl)methoxy)-7-methoxyisoquinoline-6- |
| | carboxamide 5 |
| 6 | 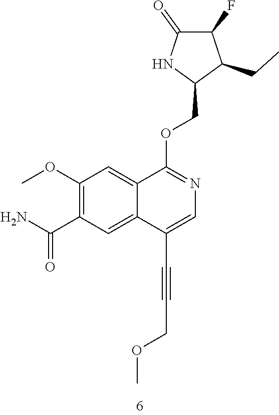 [SMILES: CC[C@@H]1[C@H](F)C(=O)N[C@@H]1COc1ncc(C#CCOC)c2cc(C(N)=O)c(OC)cc12] |
| | Example 6: 1-(((2S,3S,4S)-3-ethyl-4-fluoro-5-oxopyrrolidin-2- |
| | yl)methoxy)-7-methoxy-4-(3-methoxyprop-1-yn-1-yl)isoquinoline-6- |
| | carboxamide 6 |
| 7 | 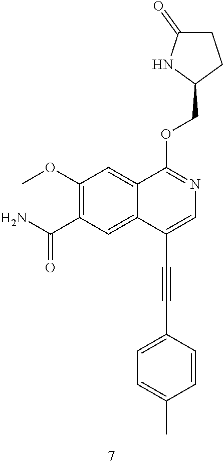 [SMILES: COc1cc2c(OC[C@@H]3CCC(=O)N3)ncc(C#Cc3ccc(C)cc3)c2cc1C(N)=O] |
| | Example 7: (S)-7-methoxy-1-((5-oxopyrrolidin-2-yl)methoxy)-4-(p- |
| | tolylethynyl)isoquinoline-6-carboxamide 7 |
| 8 | 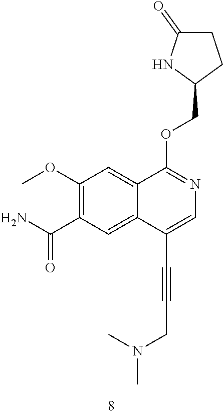 [SMILES: COc1cc2c(OC[C@@H]3CCC(=O)N3)ncc(C#CCN(C)C)c2cc1C(N)=O] |
| | Example 8: (S)-4-(3-(dimethylamino)prop-1-yn-1-yl)-7-methoxy-1-((5- |
| | oxopyrrolidin-2-yl)methoxy)isoquinoline-6-carboxamide 8 |
| 9 | 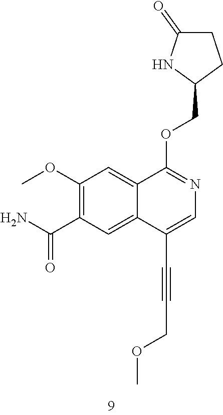 [SMILES: COCC#Cc1cnc(OC[C@@H]2CCC(=O)N2)c2cc(OC)c(C(N)=O)cc12] |
| | Example 9: (S)-7-methoxy-4-(3-methoxyprop-1-yn-1-yl)-1-((5- |
| | oxopyrrolidin-2-yl)methoxy)isoquinoline-6-carboxamide 9 |
| 10 | 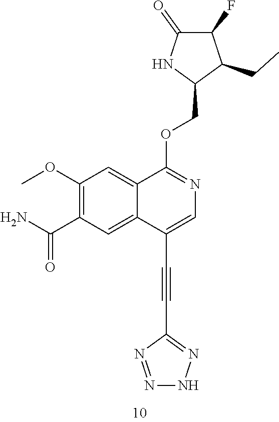 [SMILES: CC[C@@H]1[C@H](F)C(=O)N[C@@H]1COc1ncc(C#Cc2nn[nH]n2)c2cc(C(N)=O)c(OC)cc12] |
| | Example 10: 4-((2H-tetrazol-5-yl)ethynyl)-1-(((2S,3S,4S)-3-ethyl-4- |
| | fluoro-5-oxopyrrolidin-2-yl)methoxy)-7-methoxyisoquinoline-6- |
| | carboxamide 10 |
| 11 | 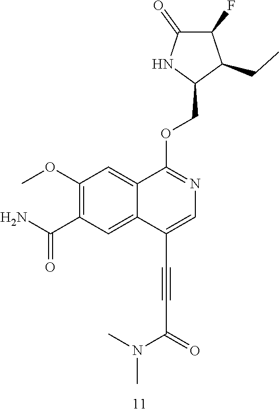 [SMILES: CC[C@@H]1[C@H](F)C(=O)N[C@@H]1COc1ncc(C#CC(=O)N(C)C)c2cc(C(N)=O)c(OC)cc12] |
| | Example 11: 4-(3-(dimethylamino)-3-oxoprop-1-yn-1-yl)-1- |
| | (((2S,3S,4S)-3-ethyl-4-fluoro-5-oxopyrrolidin-2-yl)methoxy)-7- |
| | methoxyisoquinoline-6-carboxamide 11 |
| 12 | 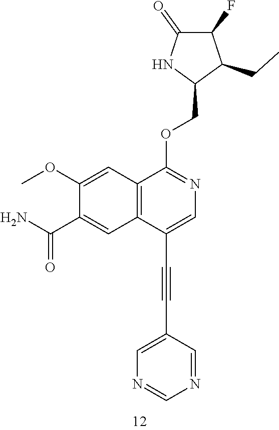 [SMILES: CC[C@@H]1[C@H](F)C(=O)N[C@@H]1COc1ncc(C#Cc2cncnc2)c2cc(C(N)=O)c(OC)cc12] |
| | Example 12: 1-(((2S,3S,4S)-3-ethyl-4-fluoro-5-oxopyrrolidin-2- |
| | yl)methoxy)-7-methoxy-4-(pyrimidin-5-ylethynyl)isoquinoline-6- |
| | carboxamide 12 |
| 13 | 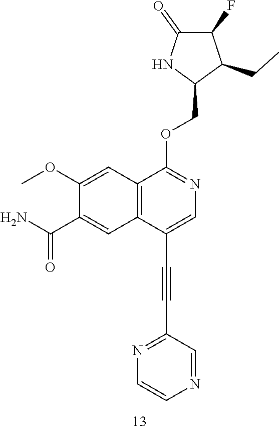 [SMILES: CC[C@@H]1[C@H](F)C(=O)N[C@@H]1COc1ncc(C#Cc2cnccn2)c2cc(C(N)=O)c(OC)cc12] |
| | Example 13: 1-(((2S,3S,4S)-3-ethyl-4-fluoro-5-oxopyrrolidin-2- |
| | yl)methoxy)-7-methoxy-4-(pyrazin-2-ylethynyl)isoquinoline-6- |
| | carboxamide 13 |
| 14 | 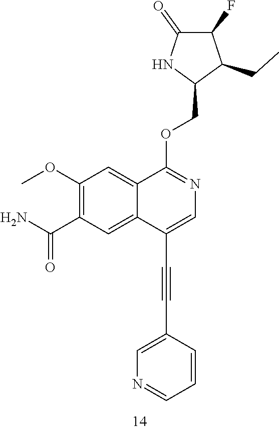 [SMILES: CC[C@@H]1[C@H](F)C(=O)N[C@@H]1COc1ncc(C#Cc2cccnc2)c2cc(C(N)=O)c(OC)cc12] |
| | Example 14: 1-(((2S,3S,4S)-3-ethyl-4-fluoro-5-oxopyrrolidin-2- |
| | yl)methoxy)-7-methoxy-4-(pyridin-3-ylethynyl)isoquinoline-6- |
| | carboxamide 14 |
| 15 | 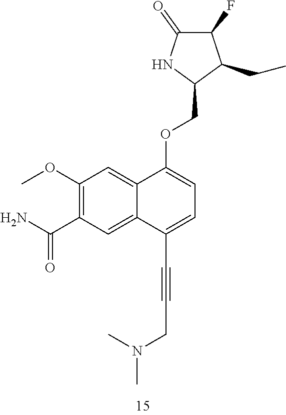 [SMILES: CC[C@@H]1[C@H](F)C(=O)N[C@@H]1COc1ccc(C#CCN(C)C)c2cc(C(N)=O)c(OC)cc12] |
| | Example 15: 8-(3-(dimethylamino)prop-1-yn-1-yl)-5-(((2S,3S,4S)-3- |
| | ethyl-4-fluoro-5-oxopyrrolidin-2-yl)methoxy)-3-methoxy-2- |
| | naphthamide 15 |
| 16 | 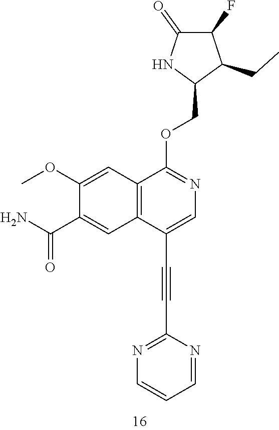 [SMILES: CC[C@@H]1[C@H](F)C(=O)N[C@@H]1COc1ncc(C#Cc2ncccn2)c2cc(C(N)=O)c(OC)cc12] |
| | Example 16: 1-(((2S,3S,4S)-3-ethyl-4-fluoro-5-oxopyrrolidin-2- |
| | yl)methoxy)-7-methoxy-4-(pyrimidin-2-ylethynyl)isoquinoline-6- |
| | carboxamide 16 |
| 17 | 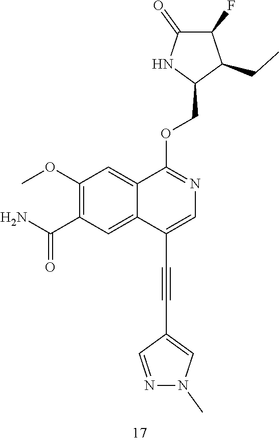 [SMILES: CC[C@@H]1[C@H](F)C(=O)N[C@@H]1COc1ncc(C#Cc2cnn(C)c2)c2cc(C(N)=O)c(OC)cc12] |
| | Example 17: 1-(((2S,3S,4S)-3-ethyl-4-fluoro-5-oxopyrrolidin-2- |
| | yl)methoxy)-7-methoxy-4-((1-methyl-1H-pyrazol-4- |
| | yl)ethynyl)isoquinoline-6-carboxamide 17 |
| 18 | 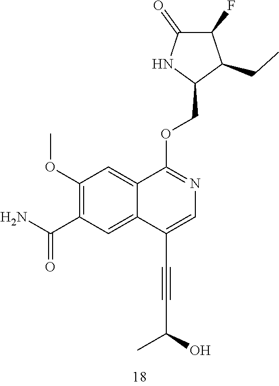 [SMILES: CC[C@@H]1[C@H](F)C(=O)N[C@@H]1COc1ncc(C#C[C@H](C)O)c2cc(C(N)=O)c(OC)cc12] |
| | Example 18: 1-(((2S,3S,4S)-3-ethyl-4-fluoro-5-oxopyrrolidin-2- |
| | yl)methoxy)-4-((S)-3-hydroxybut-1-yn-1-yl)-7-methoxyisoquinoline-6- |
| | carboxamide 18 |
| 19 | 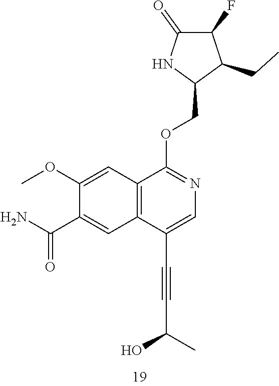 [SMILES: CC[C@@H]1[C@H](F)C(=O)N[C@@H]1COc1ncc(C#C[C@@H](C)O)c2cc(C(N)=O)c(OC)cc12] |
| | Example 19: 1-(((2S,3S,4S)-3-ethyl-4-fluoro-5-oxopyrrolidin-2- |
| | yl)methoxy)-4-((R)-3-hydroxybut-1-yn-1-yl)-7-methoxyisoquinoline-6- |
| | carboxamide 19 |
| 20 | 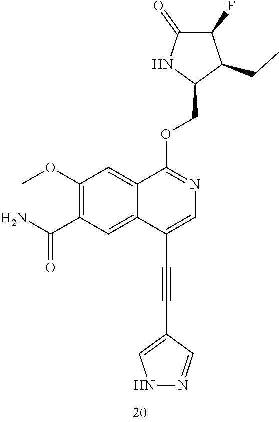 [SMILES: CC[C@@H]1[C@H](F)C(=O)N[C@@H]1COc1ncc(C#Cc2cn[nH]c2)c2cc(C(N)=O)c(OC)cc12] |
| | Example 20: 4-((1H-pyrazol-4-yl)ethynyl)-1-(((2S,3S,4S)-3-ethyl-4- |
| | fluoro-5-oxopyrrolidin-2-yl)methoxy)-7-methoxyisoquinoline-6- |
| | carboxamide 20 |
| 21 | 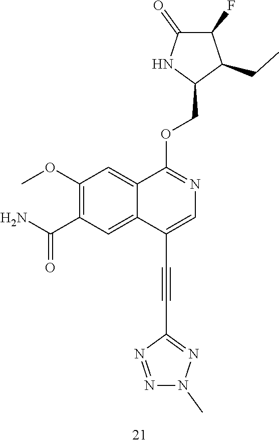 [SMILES: CC[C@@H]1[C@H](F)C(=O)N[C@@H]1COc1ncc(C#Cc2nnn(C)n2)c2cc(C(N)=O)c(OC)cc12] |
| | Example 21: 1-(((2S,3S,4S)-3-ethyl-4-fluoro-5-oxopyrrolidin-2- |
| | yl)methoxy)-7-methoxy-4-((2-methyl-2H-tetrazol-5- |
| | yl)ethynyl)isoquinoline-6-carboxamide 21 |
| 22 | 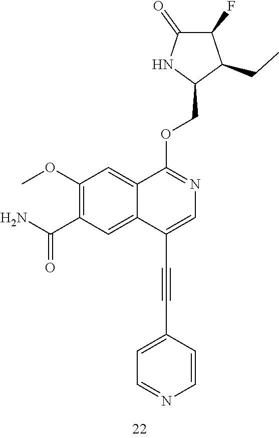 [SMILES: CC[C@@H]1[C@H](F)C(=O)N[C@@H]1COc1ncc(C#Cc2ccncc2)c2cc(C(N)=O)c(OC)cc12] |
| | Example 22: 1-(((2S,3S,4S)-3-ethyl-4-fluoro-5-oxopyrrolidin-2- |
| | yl)methoxy)-7-methoxy-4-(pyridin-4-ylethynyl)isoquinoline-6- |
| | carboxamide 22 |
| 23 | 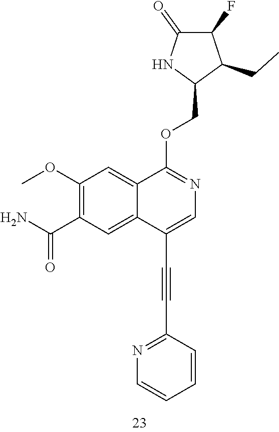 [SMILES: CC[C@@H]1[C@H](F)C(=O)N[C@@H]1COc1ncc(C#Cc2ccccn2)c2cc(C(N)=O)c(OC)cc12] |
| | Example 23: 1-(((2S,3S,4S)-3-ethyl-4-fluoro-5-oxopyrrolidin-2- |
| | yl)methoxy)-7-methoxy-4-(pyridin-2-ylethynyl)isoquinoline-6- |
| | carboxamide 23 |
| 24 | 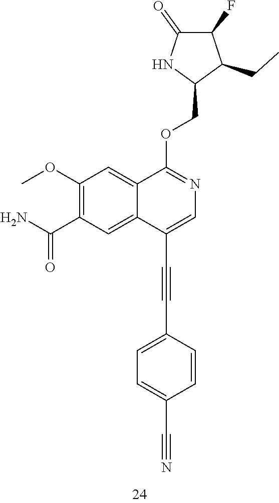 [SMILES: CC[C@@H]1[C@H](F)C(=O)N[C@@H]1COc1ncc(C#Cc2ccc(C#N)cc2)c2cc(C(N)=O)c(OC)cc12] |
| | Example 24: 4-((4-cyanophenyl)ethynyl)-1-(((2S,3S,4S)-3-ethyl-4- |
| | fluoro-5-oxopyrrolidin-2-yl)methoxy)-7-methoxyisoquinoline-6- |
| | carboxamide 24 |
| 25 | 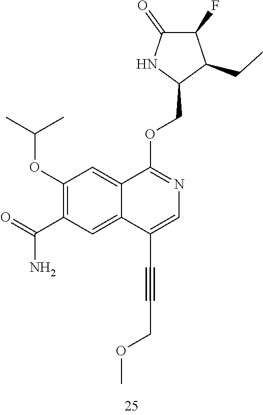 [SMILES: CC[C@@H]1[C@H](F)C(=O)N[C@@H]1COc1ncc(C#CCOC)c2cc(C(N)=O)c(OC(C)C)cc12] |
| | Example 25: 1-(((2S,3S,4S)-3-ethyl-4-fluoro-5-oxopyrrolidin-2- |
| | yl)methoxy)-7-isopropoxy-4-(3-methoxyprop-1-yn-1-yl)isoquinoline-6- |
| | carboxamide 25 |
| 26 | 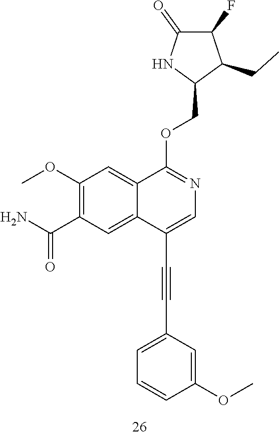 [SMILES: CC[C@@H]1[C@H](F)C(=O)N[C@@H]1COc1ncc(C#Cc2cccc(OC)c2)c2cc(C(N)=O)c(OC)cc12] |
| | Example 26: 1-(((2S,3S,4S)-3-ethyl-4-fluoro-5-oxopyrrolidin-2- |
| | yl)methoxy)-7-methoxy-4-((3-methoxyphenyl)ethynyl)isoquinoline-6- |
| | carboxamide 26 |
| 27 | 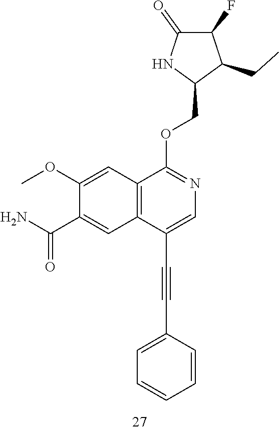 [SMILES: CC[C@@H]1[C@H](F)C(=O)N[C@@H]1COc1ncc(C#Cc2ccccc2)c2cc(C(N)=O)c(OC)cc12] |
| | Example 27: 1-(((2S,3S,4S)-3-ethyl-4-fluoro-5-oxopyrrolidin-2- |
| | yl)methoxy)-7-methoxy-4-(phenylethynyl)isoquinoline-6-carboxamide |
| | 27 |
| 28 | 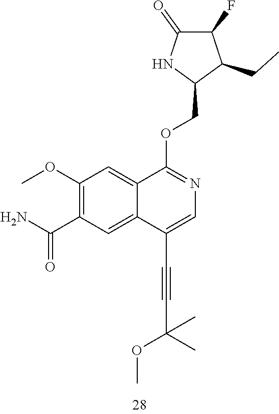 [SMILES: CC[C@@H]1[C@H](F)C(=O)N[C@@H]1COc1ncc(C#CC(C)(C)OC)c2cc(C(N)=O)c(OC)cc12] |
| | Example 28: 1-(((2S,3S,4S)-3-ethyl-4-fluoro-5-oxopyrrolidin-2- |
| | yl)methoxy)-7-methoxy-4-(3-methoxy-3-methylbut-1-yn-1- |
| | yl)isoquinoline-6-carboxamide 28 |
| 29 | 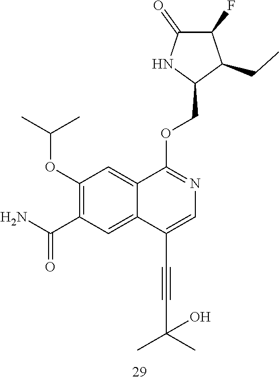 [SMILES: CC[C@@H]1[C@H](F)C(=O)N[C@@H]1COc1ncc(C#CC(C)(C)O)c2cc(C(N)=O)c(OC(C)C)cc12] |
| | Example 29: 1-(((2S,3S,4S)-3-ethyl-4-fluoro-5-oxopyrrolidin-2- |
| | yl)methoxy)-4-(3-hydroxyl-3-methylbut-1-yn-1-yl)-7- |
| | isopropoxyisoquinoline-6-carboxamide 29 |
| 30 | 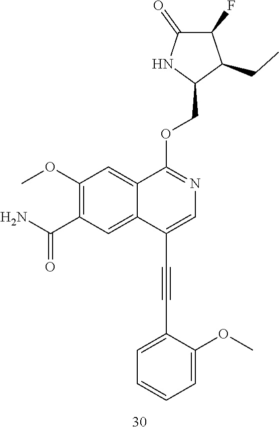 [SMILES: CC[C@@H]1[C@H](F)C(=O)N[C@@H]1COc1ncc(C#Cc2ccccc2OC)c2cc(C(N)=O)c(OC)cc12] |
| | Example 30: 1-(((2S,3S,4S)-3-ethyl-4-fluoro-5-oxopyrrolidin-2- |
| | yl)methoxy)-7-methoxy-4-((2-methoxyphenyl)ethynyl)isoquinoline-6- |
| | carboxamide 30 |
| 31 | 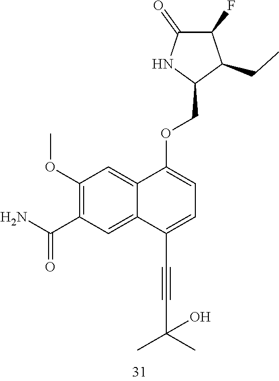 [SMILES: CC[C@@H]1[C@H](F)C(=O)N[C@@H]1COc1ccc(C#CC(C)(C)O)c2cc(C(N)=O)c(OC)cc12] |
| | Example 31: 5-(((2S,3S,4S)-3-ethyl-4-fluoro-5-oxopyrrolidin-2- |
| | yl)methoxy)-8-(3-hydroxyl-3-methylbut-1-yn-1-yl)-3-methoxy-2- |
| | naphthamide 31 |
| 32 | 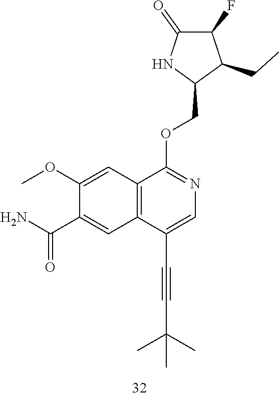 [SMILES: CC[C@@H]1[C@H](F)C(=O)N[C@@H]1COc1ncc(C#CC(C)(C)C)c2cc(C(N)=O)c(OC)cc12] |
| | Example 32: 4-(3,3-dimethylbut-1-yn-1-yl)-1-(((2S,3S,4S)-3-ethyl-4- |
| | fluoro-5-oxopyrrolidin-2-yl)methoxy)-7-methoxyisoquinoline-6- |
| | carboxamide 32 |
| 33 | 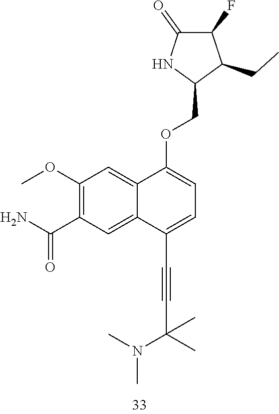 [SMILES: CC[C@@H]1[C@H](F)C(=O)N[C@@H]1COc1ccc(C#CC(C)(C)N(C)C)c2cc(C(N)=O)c(OC)cc12] |
| | Example 33: 8-(3-(dimethylamino)-3-methylbut-1-yn-1-yl)-5- |
| | (((2S,3S,4S)-3-ethyl-4-fluoro-5-oxopyrrolidin-2-yl)methoxy)-3- |
| | methoxy-2-naphthamide 33 |
| 34 | 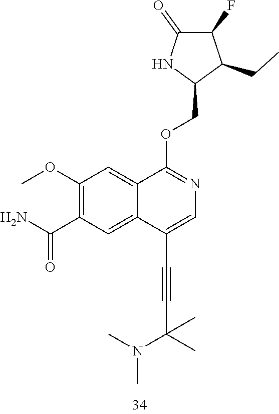 [SMILES: CC[C@@H]1[C@H](F)C(=O)N[C@@H]1COc1ncc(C#CC(C)(C)N(C)C)c2cc(C(N)=O)c(OC)cc12] |
| | Example 34: 4-(3-(dimethylamino)-3-methylbut-1-yn-1-yl)-1- |
| | (((2S,3S,4S)-3-ethyl-4-fluoro-5-oxopyrrolidin-2-yl)methoxy)-7- |
| | methoxyisoquinoline-6-carboxamide 34 |
| 35 | 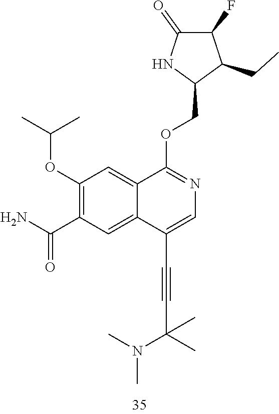 [SMILES: CC[C@@H]1[C@H](F)C(=O)N[C@@H]1COc1ncc(C#CC(C)(C)N(C)C)c2cc(C(N)=O)c(OC(C)C)cc12] |
| | Example 35: 4-(3-(dimethylamino)-3-methylbut-1-yn-1-yl)-1- |
| | (((2S,3S,4S)-3-ethyl-4-fluoro-5-oxopyrrolidin-2-yl)methoxy)-7- |
| | isopropoxyisoquinoline-6-carboxamide 35 |
| 4-3 | 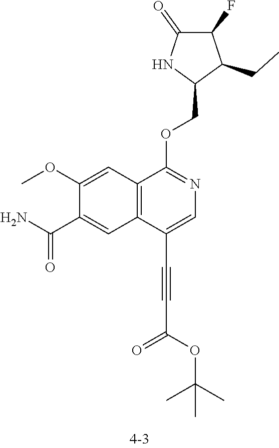 [SMILES: CC[C@@H]1[C@H](F)C(=O)N[C@@H]1COc1ncc(C#CC(=O)OC(C)(C)C)c2cc(C(N)=O)c(OC)cc12] |
| | tert-butyl 3-(6-carbamoyl-1-(((2S,3S,4S)-3-ethyl-4-fluoro-5- |
| | oxopyrrolidin-2-yl)methoxy)-7-methoxyisoquinolin-4-yl)propiolate 4-3 |
| 20-5 | 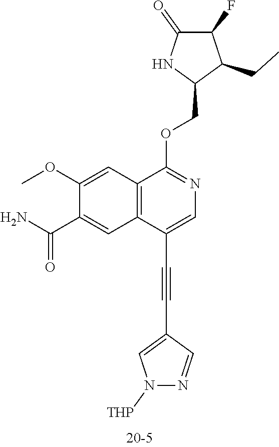 |
| | 1-(((2S,3S,4S)-3-ethyl-4-fluoro-5-oxopyrrolidin-2-yl)methoxy)-7- |
| | methoxy-4-((1-(tetrahydro-2H-pyran-2-yl)-1H-pyrazol-4- |
| | yl)ethynyl)isoquinoline-6-carboxamide 20-5 |

or a tautomer, mesomer, racemate, enantiomer, diastereomer, or mixture thereof, or a pharmaceutically acceptable salt thereof.

In one aspect, the present disclosure provides a compound of formula (IA1), or a tautomer, mesomer, racemate, enantiomer, diastereomer, or mixture thereof, or pharmaceutically acceptable salt thereof:

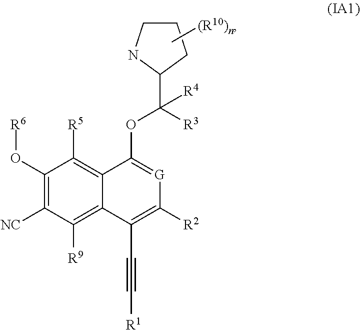

wherein G, R¹-R⁶, R⁹, R¹⁰ and n are each as defined in formula (I).

In one aspect, the present disclosure provides a compound of formula (IA2), or a tautomer, mesomer, racemate, enantiomer, diastereomer, or mixture thereof, or pharmaceutically acceptable salt thereof:

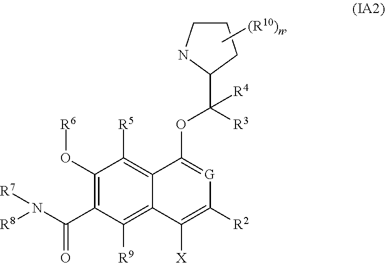

wherein:

X is halogen or OTf; and

G, R²-R¹⁰ and n are each as defined in formula (I).

In one aspect, the present disclosure provides a compound of formula (IIA1), or a tautomer, mesomer, racemate, enantiomer, diastereomer, or mixture thereof, or pharmaceutically acceptable salt thereof:

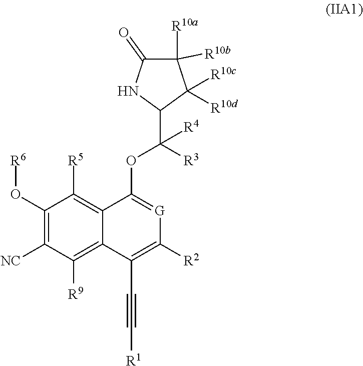

wherein

G, R¹-R⁶, R⁹, R^{10a}, R^{10b}, R^{10c} and R^{10d} are each as defined in formula (II).

In one aspect, the present disclosure provides a compound of formula (IIA2), or a tautomer, mesomer, racemate, enantiomer, diastereomer, or mixture thereof, or pharmaceutically acceptable salt thereof:

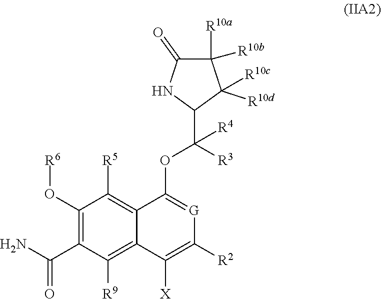

wherein X is Cl, Br, I, or OTf;

G is CR^{g}; and

R^{g}, R²-R⁶, R⁹, and R^{10a}-R^{10d} are each as defined in formula (II).

In one aspect, the present disclosure provides a compound of formula (IIIA1), or a tautomer, mesomer, racemate, enantiomer, diastereomer, or mixture thereof, or pharmaceutically acceptable salt thereof:

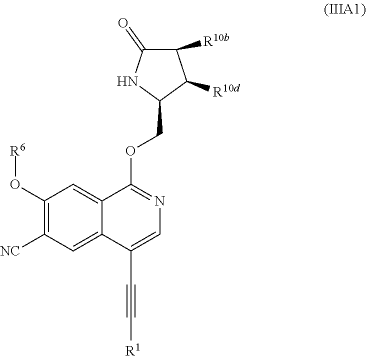

wherein

R¹, R⁶, R^{10b} and R^{10d} are each as defined in formula (III).

In one aspect, the present disclosure provides a compound of formula (IIIA2), or a tautomer, mesomer, racemate, enantiomer, diastereomer, or mixture thereof, or pharmaceutically acceptable salt thereof:

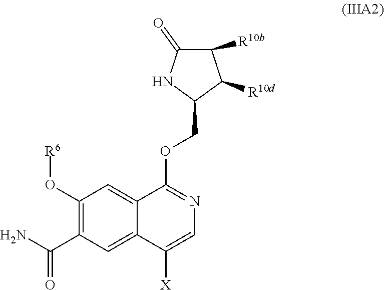

wherein X is halogen or OTf; and

R⁶, R^{10b} and R^{10d} are each as defined in formula (III).

Exemplified compounds of the disclosure include, but are not limited to:

| Example | |
|---|---|
| No. | Compound structure and name |
| 1f | 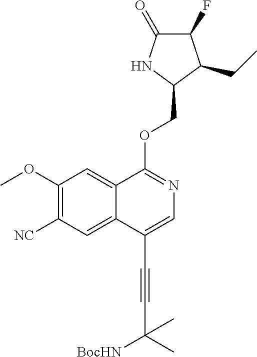 [SMILES: CC[C@@H]1[C@H](F)C(=O)N[C@@H]1COc1ncc(C#CC(C)(C)NC(=O)OC(C)(C)C)c2cc(C#N)c(OC)cc12] |
| | 1f |
| | tert-butyl (4-(6-cyano-1-(((2S,3S,4S)-3- |
| | ethyl-4-fluoro-5-oxopyrrolidin-2-yl)methoxy)- |
| | 7-methoxyisoquinolin-4-yl)-2-methylbut-3-yn-2- |
| | yl)carbamate 1f |
| 3b | 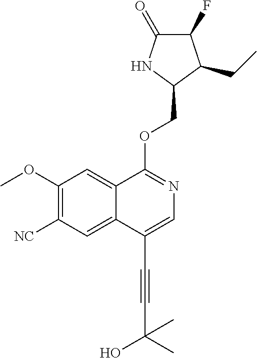 [SMILES: CC[C@@H]1[C@H](F)C(=O)N[C@@H]1COc1ncc(C#CC(C)(C)O)c2cc(C#N)c(OC)cc12] |
| | 3b |
| | 1-(((2S,3S,4S)-3-ethyl-4-fluoro-5- |
| | oxopyrrolidin-2-yl)methoxy)-4-(3-hydroxyl- |
| | 3-methylbut-1-yn-1-yl)-7- |
| | methoxyisoquinoline-6-carbonitrile 3b |
| 4-2 | 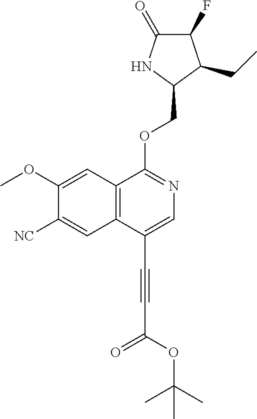 [SMILES: CC[C@@H]1[C@H](F)C(=O)N[C@@H]1COc1ncc(C#CC(=O)OC(C)(C)C)c2cc(C#N)c(OC)cc12] |
| | 4-2 |
| | tert-butyl 3-(6-cyano-1-(((2S,3S,4S)-3-ethyl-4- |
| | fluoro-5-oxopyrrolidin-2-yl)methoxy)-7- |
| | methoxyisoquinolin-4-yl)propiolate 4-2 |
| | 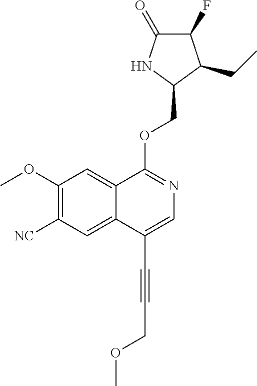 [SMILES: CC[C@@H]1[C@H](F)C(=O)N[C@@H]1COc1ncc(C#CCOC)c2cc(C#N)c(OC)cc12] |
| 7d | 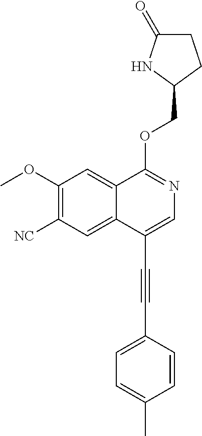 [SMILES: COc1cc2c(OC[C@@H]3CCC(=O)N3)ncc(C#Cc3ccc(C)cc3)c2cc1C#N] |
| | 7d |
| | (S)-7-methoxy-1-((5-oxopyrrolidin- |
| | 2-yl)methoxy)-4-(p-tolylethynyl) |
| | isoquinoline-6-carbonitrile 7d |
| | 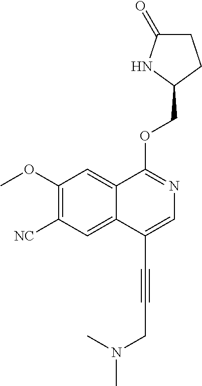 [SMILES: COc1cc2c(OC[C@@H]3CCC(=O)N3)ncc(C#CCN(C)C)c2cc1C#N] |
| | 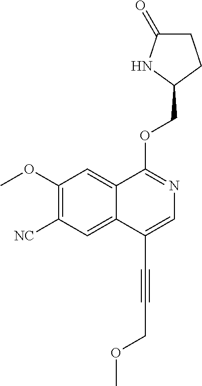 [SMILES: COCC#Cc1cnc(OC[C@@H]2CCC(=O)N2)c2cc(OC)c(C#N)cc12] |
| | 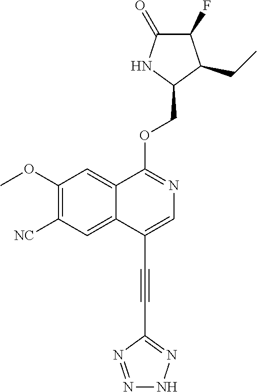 [SMILES: CC[C@@H]1[C@H](F)C(=O)N[C@@H]1COc1ncc(C#Cc2nn[nH]n2)c2cc(C#N)c(OC)cc12] |
| | 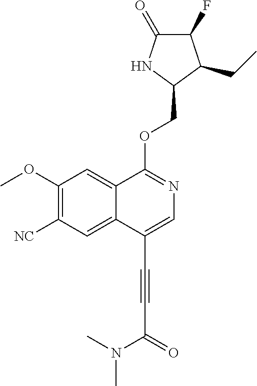 [SMILES: CC[C@@H]1[C@H](F)C(=O)N[C@@H]1COc1ncc(C#CC(=O)N(C)C)c2cc(C#N)c(OC)cc12] |
| 12-2 | 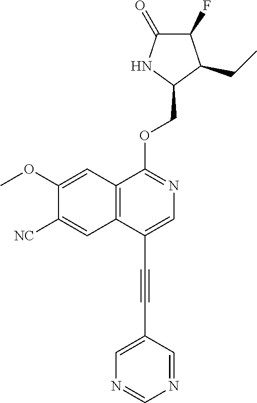 [SMILES: CC[C@@H]1[C@H](F)C(=O)N[C@@H]1COc1ncc(C#Cc2cncnc2)c2cc(C#N)c(OC)cc12] |
| | 12-2 |
| | 1-(((2S,3S,4S)-3-ethyl-4-fluoro-5- |
| | oxopyrrolidin-2-yl)methoxy)-7-methoxy-4- |
| | (pyrimidin-5-ylethynyl)isoquinoline-6- |
| | carbonitrile 12-2 |
| | 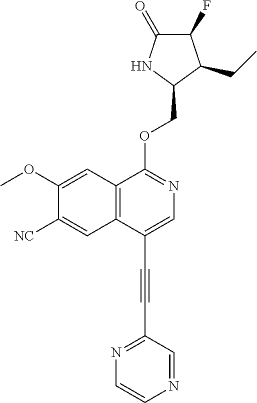 [SMILES: CC[C@@H]1[C@H](F)C(=O)N[C@@H]1COc1ncc(C#Cc2cnccn2)c2cc(C#N)c(OC)cc12] |
| | 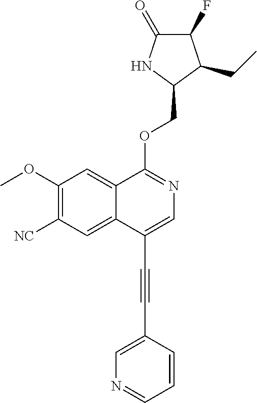 [SMILES: CC[C@@H]1[C@H](F)C(=O)N[C@@H]1COc1ncc(C#Cc2cccnc2)c2cc(C#N)c(OC)cc12] |
| 15e | 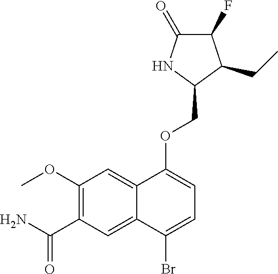 [SMILES: CC[C@@H]1[C@H](F)C(=O)N[C@@H]1COc1ccc(Br)c2cc(C(N)=O)c(OC)cc12] |
| | 15e |
| | 8-bromo-5-(((2S,3S,4S)-3-ethyl-4-fluoro-5- |
| | oxopyrrolidin-2-yl)methoxy)-3-methoxy- |
| | 2-naphthamide 15e |
| | 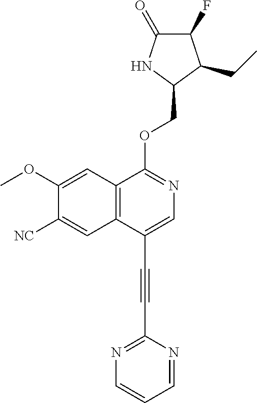 [SMILES: CC[C@@H]1[C@H](F)C(=O)N[C@@H]1COc1ncc(C#Cc2ncccn2)c2cc(C#N)c(OC)cc12] |
| | 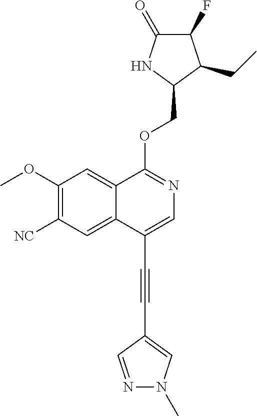 [SMILES: CC[C@@H]1[C@H](F)C(=O)N[C@@H]1COc1ncc(C#Cc2cnn(C)c2)c2cc(C#N)c(OC)cc12] |
| | 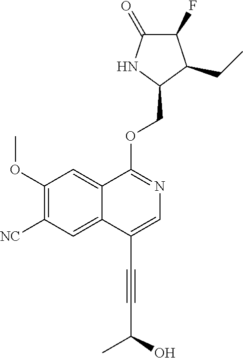 [SMILES: CC[C@@H]1[C@H](F)C(=O)N[C@@H]1COc1ncc(C#C[C@H](C)O)c2cc(C#N)c(OC)cc12] |
| | 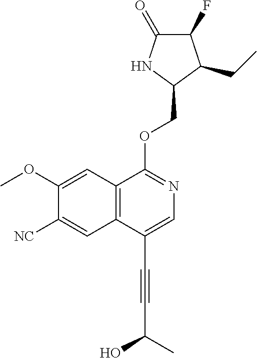 [SMILES: CC[C@@H]1[C@H](F)C(=O)N[C@@H]1COc1ncc(C#C[C@@H](C)O)c2cc(C#N)c(OC)cc12] |
| 20-4 | 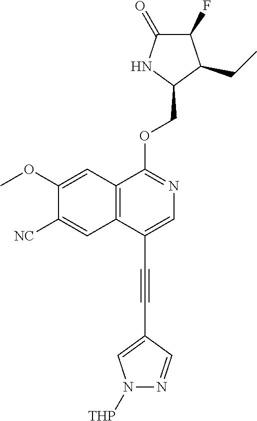 |
| | 20-4 |
| | 1-(((2S,3S,4S)-3-ethyl-4-fluoro-5- |
| | oxopyrrolidin-2-yl)methoxy)-7-methoxy-4- |
| | ((1-(tetrahydro-2H-pyran-2-yl)-1H-pyrazol- |
| | 4-yl)ethynyl)isoquinoline-6-carbonitrile 20-4 |
| | 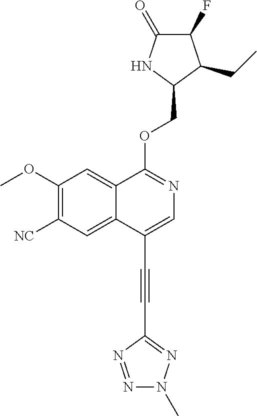 [SMILES: CC[C@@H]1[C@H](F)C(=O)N[C@@H]1COc1ncc(C#Cc2nnn(C)n2)c2cc(C#N)c(OC)cc12] |
| | 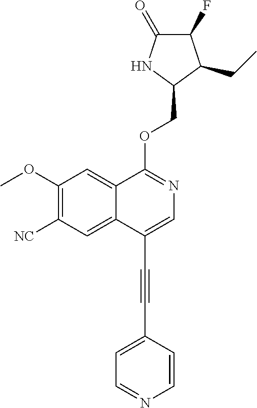 [SMILES: CC[C@@H]1[C@H](F)C(=O)N[C@@H]1COc1ncc(C#Cc2ccncc2)c2cc(C#N)c(OC)cc12] |
| | 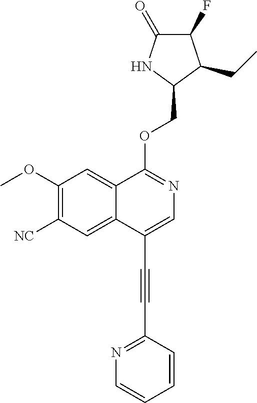 [SMILES: CC[C@@H]1[C@H](F)C(=O)N[C@@H]1COc1ncc(C#Cc2ccccn2)c2cc(C#N)c(OC)cc12] |
| | 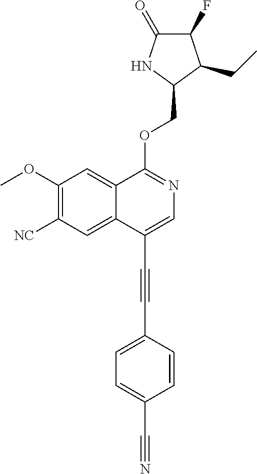 [SMILES: CC[C@@H]1[C@H](F)C(=O)N[C@@H]1COc1ncc(C#Cc2ccc(C#N)cc2)c2cc(C#N)c(OC)cc12] |
| 25e | 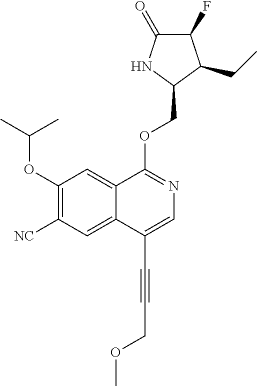 [SMILES: CC[C@@H]1[C@H](F)C(=O)N[C@@H]1COc1ncc(C#CCOC)c2cc(C#N)c(OC(C)C)cc12] |
| | 25e |
| | 1-(((2S,3S,4S)-3-ethyl-4-fluoro-5- |
| | oxopyrrolidin-2-yl)methoxy)-7-isopropoxy- |
| | 4-(3-methoxyprop-1-yn-1-yl)isoquinoline- |
| | 6-carbonitrile 25e |
| | 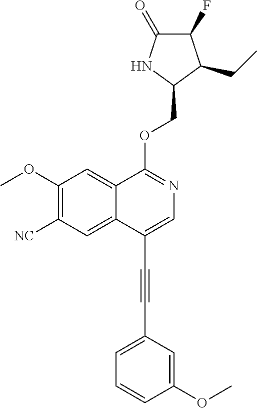 [SMILES: CC[C@@H]1[C@H](F)C(=O)N[C@@H]1COc1ncc(C#Cc2cccc(OC)c2)c2cc(C#N)c(OC)cc12] |
| | 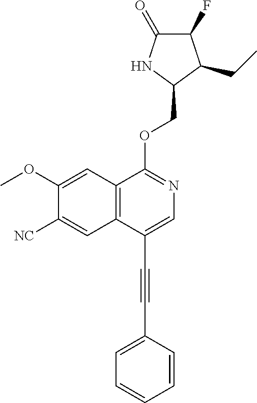 [SMILES: CC[C@@H]1[C@H](F)C(=O)N[C@@H]1COc1ncc(C#Cc2ccccc2)c2cc(C#N)c(OC)cc12] |
| | 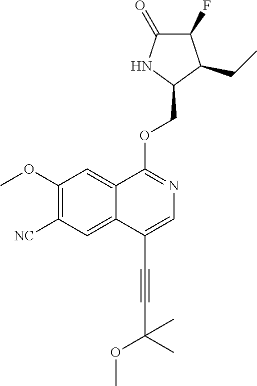 [SMILES: CC[C@@H]1[C@H](F)C(=O)N[C@@H]1COc1ncc(C#CC(C)(C)OC)c2cc(C#N)c(OC)cc12] |
| | 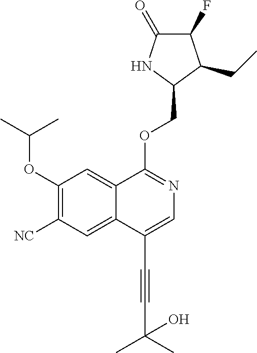 [SMILES: CC[C@@H]1[C@H](F)C(=O)N[C@@H]1COc1ncc(C#CC(C)(C)O)c2cc(C#N)c(OC(C)C)cc12] |
| | 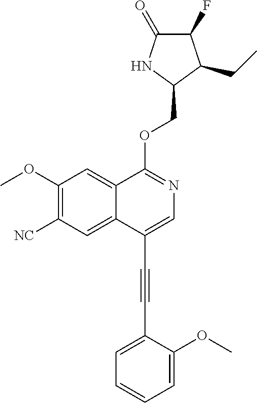 [SMILES: CC[C@@H]1[C@H](F)C(=O)N[C@@H]1COc1ncc(C#Cc2ccccc2OC)c2cc(C#N)c(OC)cc12] |
| | 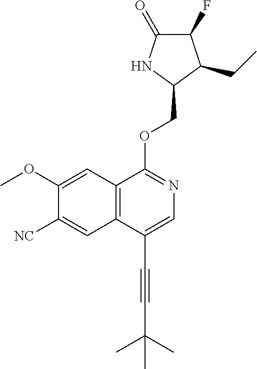 [SMILES: CC[C@@H]1[C@H](F)C(=O)N[C@@H]1COc1ncc(C#CC(C)(C)C)c2cc(C#N)c(OC)cc12] |
| | 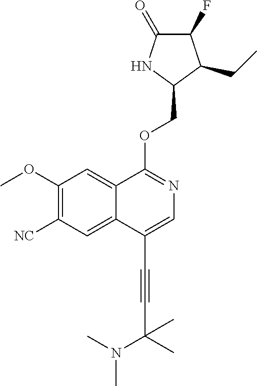 [SMILES: CC[C@@H]1[C@H](F)C(=O)N[C@@H]1COc1ncc(C#CC(C)(C)N(C)C)c2cc(C#N)c(OC)cc12] |
| | 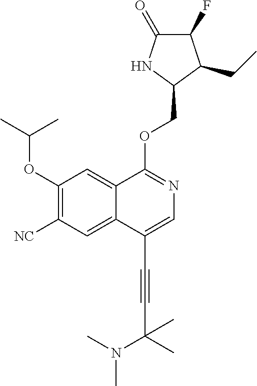 [SMILES: CC[C@@H]1[C@H](F)C(=O)N[C@@H]1COc1ncc(C#CC(C)(C)N(C)C)c2cc(C#N)c(OC(C)C)cc12] |

or a tautomer, mesomer, racemate, enantiomer, diastereomer, or mixture thereof, or a pharmaceutically acceptable salt thereof.

In another aspect, this disclosure provides a preparation process of a compound of formula (I), or a tautomer, mesomer, racemate, enantiomer, diastereomer, or mixture thereof, or a pharmaceutically acceptable salt thereof, the preparation process comprising the steps of:

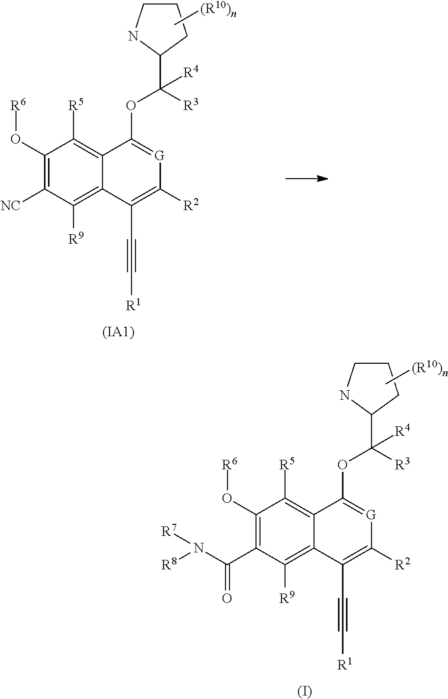

the compound of formula (IA1) is subjected to hydration reaction to obtain the compound of formula (I), wherein:

R⁷ is hydrogen;

R⁸ is hydrogen; and

G, R¹ to R⁶, R⁹, R¹⁰ and n are each as defined in formula (I).

In another aspect, this disclosure provides a preparation process of a compound of formula (I), or a tautomer, mesomer, racemate, enantiomer, diastereomer, or mixture thereof, or a pharmaceutically acceptable salt thereof, the preparation process comprising the steps of:

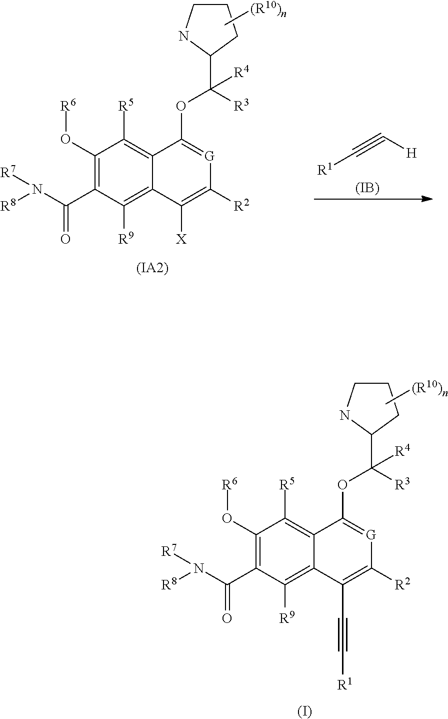

reacting a compound of formula (IA2) with a compound of formula (IB) to obtain the compound of formula (I), wherein:

X is halogen or OTf;

G is N or CR^{g}; preferably is CR^{g}; and

R^{g}, R¹ to R¹⁰ and n are each as defined in formula (I).

In another aspect, this disclosure provides a preparation process of a compound of formula (II), or a tautomer, mesomer, racemate, enantiomer, diastereomer, or mixture thereof, or a pharmaceutically acceptable salt thereof, the preparation process comprising the steps of:

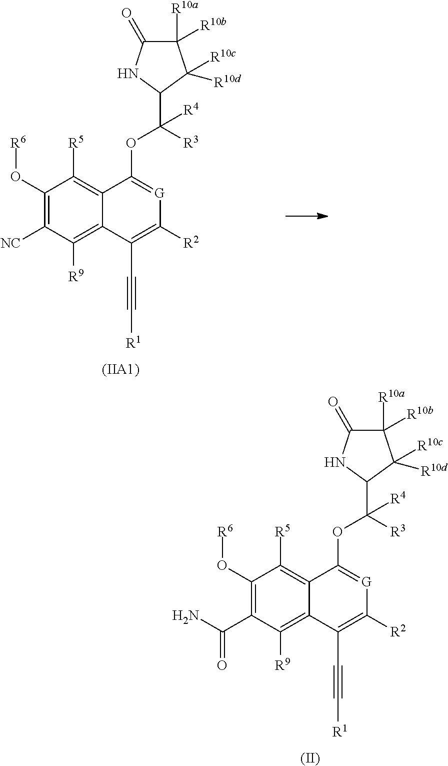

the compound of formula (IIA1) is subjected to hydration reaction to obtain the compound of formula (II), wherein:

G, R¹-R⁶, R⁹, R^{10a}, R^{10b}, R^{10c} and R^{10d} are each as defined in formula (II).

In another aspect, this disclosure provides a preparation process of a compound of formula (II), or a tautomer, mesomer, racemate, enantiomer, diastereomer, or mixture thereof, or a pharmaceutically acceptable salt thereof, the preparation process comprising the steps of:

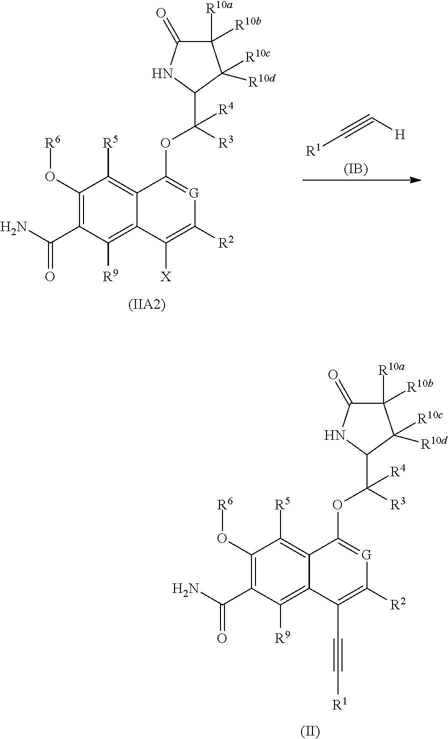

reacting a compound of formula (IIA2) with a compound of formula (IB) to obtain the compound of formula (II), wherein:

X is Cl, Br, I, or OTf;

G is N or CR^{g}; preferably is CR^{g}; and

R^{g}, R¹-R⁶, R⁹, R^{10a}, R^{10b}, R^{10c} and R^{10d} are each as defined in formula (II).

In another aspect, this disclosure provides a preparation process of a compound of formula (III), or a tautomer, mesomer, racemate, enantiomer, diastereomer, or mixture thereof, or a pharmaceutically acceptable salt thereof, the preparation process comprising the steps of:

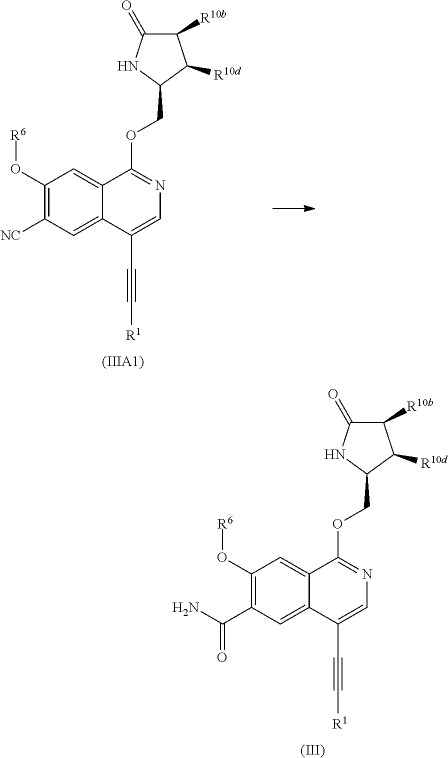

the compound of formula (IIIA1) is subjected to hydration reaction to obtain the compound of formula (III), wherein:

R¹, R⁶, R^{10b} and R^{10d} are each as defined in formula (III).

In another aspect, this disclosure provides a preparation process of a compound of formula (III), or a tautomer, mesomer, racemate, enantiomer, diastereomer, or mixture thereof, or a pharmaceutically acceptable salt thereof, the preparation process comprising the steps of:

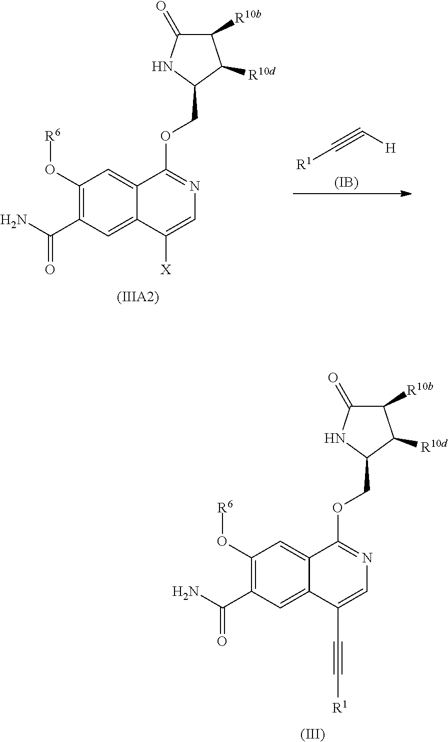

reacting a compound of formula (IIIA2) with a compound of formula (IB) to obtain the compound of formula (III), wherein:

X is halogen or OTf; and

R¹, R⁶, R^{10b} and R^{10d} are each as defined in formula (III).

The present disclosure also provides a pharmaceutical composition, comprising a compound of formula (I), or a tautomer, mesomer, racemate, enantiomer, diastereomer, or mixture thereof, or a pharmaceutically acceptable salt thereof, and a pharmaceutically acceptable carrier.

The present disclosure also provides a pharmaceutical composition, comprising a therapeutically effective amount of a compound of formula (I), or a tautomer, mesomer, racemate, enantiomer, diastereomer, or mixture thereof, or a pharmaceutically acceptable salt thereof, together with one or more pharmaceutically acceptable carriers, diluents or excipients.

The present disclosure relates to a method of inhibiting IRAK protein kinase, comprising administering to a subject in need thereof a therapeutically effective amount of the compound of formula (I), or a tautomer, mesomer, racemate, enantiomer, diastereomer, or mixture thereof, or a pharmaceutically acceptable salt thereof, or the pharmaceutical composition containing the same; preferably the IRAK protein kinase is an IRAK-4 protein kinase.

The present disclosure relates to a method of preventing and/or treating IRAK-mediated disorder, disease, or condition, comprising a step of administering to a subject in need thereof a therapeutically effective amount of the compound of formula (I), or a tautomer, mesomer, racemate, enantiomer, diastereomer, or mixture thereof, or a pharmaceutically acceptable salt thereof, or pharmaceutical composition containing the same.

The present disclosure relates to a method of preventing and/or treating a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder, comprising a step of administering to a subject in need thereof a therapeutically effective amount of the compound of formula (I), or a tautomer, mesomer, racemate, enantiomer, diastereomer, or mixture thereof, or a pharmaceutically acceptable salt thereof, or pharmaceutical composition containing the same.

In other words, the present disclosure also relates to use of a compound of formula (I), or a tautomer, mesomer, racemate, enantiomer, diastereomer, or mixture thereof, or a pharmaceutically acceptable salt thereof, or pharmaceutical composition containing the same, in the preparation of a medicament for the inhibition of IRAK protein kinase; preferably the IRAK protein kinase is an IRAK-4 protein kinase.

In other words, the present disclosure also relates to use of a compound of formula (I) or a tautomer, mesomer, racemate, enantiomer, diastereomer, or mixture thereof, or a pharmaceutically acceptable salt thereof, or pharmaceutical composition containing the same, in the preparation of a medicament for preventing and/or treating IRAK-mediated disorder, disease, or condition.

In other words, the present disclosure also relates to use of a compound of formula (I) or a tautomer, mesomer, racemate, enantiomer, diastereomer, or mixture thereof, or a pharmaceutically acceptable salt thereof, or pharmaceutical composition containing the same, in the preparation of a medicament for preventing and/or treating a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

The present disclosure further relates to the compound of formula (I), or a tautomer, mesomer, racemate, enantiomer, diastereomer, or mixture thereof, or a pharmaceutically acceptable salt thereof, or pharmaceutical composition containing the same, for use as a medicament.

The present disclosure also relates to the compound of formula (I), or a tautomer, mesomer, racemate, enantiomer, diastereomer, or mixture thereof, or a pharmaceutically acceptable salt thereof, or pharmaceutical composition containing the same, for use in inhibiting IRAK protein kinase; preferably the IRAK protein kinase is an IRAK-4 protein kinase.

The present disclosure also relates to the combination of the compound of formula (I), or a tautomer, mesomer, racemate, enantiomer, diastereomer, or mixture thereof, or a pharmaceutically acceptable salt thereof, or pharmaceutical composition containing the same, for use in preventing and/or treating IRAK-mediated disorder, disease, or condition.

The present disclosure also relates to the combination of the compound of formula (I), or a tautomer, mesomer, racemate, enantiomer, diastereomer, or mixture thereof, or a pharmaceutically acceptable salt thereof, or pharmaceutical composition containing the same, for use in preventing and/or treating a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

The “cancer” or “proliferative disorder” mentioned includes a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, nonsmall-cell lung carcinoma, lymphomas, Hodgkins and Non-Hodgkins, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABCDLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulmemia (WM), splenic marginal zone lymphoma, multiple myeloma, plasmacytoma, and intravascular large B-cell lymphoma).

The “MyD88 driven disorder” mentioned includes ABC DLBCL, Waldenstrom's macroglobulmemia, Hodgkin's lymphoma, primary cutaneous T-cell lymphoma and chronic lymphocytic leukemia; and/or the IL-1 driven disorder is Smoldering of indolent multiple myeloma.

The “IL-1” driven disorder mentioned is Smoldering of indolent multiple myeloma.

The “neurodegenerative disease” mentioned includes Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

The “inflammatory disorder” refers to conditions of the eye such as ocular allergy, conjunctivitis, keratoconjunctivitis sicca, and vernal conjunctivitis; diseases affecting the nose including allergic rhinitis; and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Juvenile rheumatoid arthritis, Cryopyrin Associated Periodic Syndrome (CAPS), and osteoarthritis.

The compositions of this disclosure can be formulated by conventional methods using one or more pharmaceutically acceptable carriers. Thus, the active compounds of this disclosure can be formulated as various dosage forms for oral, buccal, intranasal, parenteral (e.g., intravenous, intramuscular or subcutaneous), rectal administration, inhalation or insufflation administration. The compounds of this disclosure can also be formulated as sustained release dosage forms.

a tablet, troche, lozenge, aqueous or oily suspension, dispersible powder or granule, emulsion, hard or soft capsule, or syrup or elixir. Oral compositions can be prepared according to any known method in the art for the preparation of pharmaceutical compositions. Such compositions can contain one or more additives selected from sweeteners, flavoring agents, colorants and preservatives, in order to provide a pleasing and palatable pharmaceutical preparation. Tablets contain the active ingredient and nontoxic pharmaceutically acceptable excipients suitable for the manufacture of tablets. These excipients can be inert excipients, granulating agents, disintegrating agents, and lubricants. The tablet can be uncoated or coated by means of a known technique to mask the taste of the drug or delay the disintegration and absorption of the drug in the gastrointestinal tract, thereby providing sustained release over an extended period. For example, water soluble taste masking materials can be used.

Oral formulations can also be provided as soft gelatin capsules in which the active ingredient is mixed with an inert solid diluent, or the active ingredient is mixed with a water soluble carrier.

An aqueous suspension contains the active ingredient in admixture with excipients suitable for the manufacture of an aqueous suspension. Such excipients are suspending agents, dispersants or humectants, and can be naturally occurring phospholipids. The aqueous suspension can also contain one or more preservatives, one or more colorants, one or more flavoring agents, and one or more sweeteners.

An oil suspension can be formulated by suspending the active ingredient in a vegetable oil, or in a mineral oil. The oil suspension can contain a thickener. The aforementioned sweeteners and flavoring agents can be added to provide a palatable preparation. These compositions can be preserved by adding an antioxidant.

The active ingredient and the dispersants or wetting agents, suspending agent or one or more preservatives can be prepared as a dispersible powder or granule suitable for the preparation of an aqueous suspension by adding water. Suitable dispersants or wetting agents and suspending agents are exemplified by those already mentioned above. Additional excipients, such as sweeteners, flavoring agents and colorants, can also be added. These compositions can be preserved by adding an antioxidant such as ascorbic acid.

The present pharmaceutical composition can also be in the form of an oil-in-water emulsion. The oil phase can be a vegetable oil, or a mineral oil, or mixture thereof. Suitable emulsifying agents can be naturally occurring phospholipids. Sweeteners can be used. Such formulations can also contain moderators, preservatives, colorants and antioxidants.

The pharmaceutical composition can be in the form of a sterile injectable aqueous solution. The acceptable vehicles and solvents that can be employed are water, Ringer's solution and isotonic sodium chloride solution. The sterile injectable preparation can also be a sterile injectable oil-in-water microemulsion in which the active ingredient is dissolved in the oil phase. The injectable solution or microemulsion can be introduced into an individual's bloodstream by local bolus injection. Alternatively, it can be advantageous to administer the solution or microemulsion in such a way as to maintain a constant circulating concentration of the present compound. In order to maintain such a constant concentration, a continuous intravenous delivery device can be utilized. An example of such a device is Deltec CADD-PLUS™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable preparation can also be a sterile injectable solution or suspension prepared in a nontoxic parenterally acceptable diluent or solvent. Moreover, sterile fixed oils can easily be used as a solvent or suspending medium, and fatty acids can also be used to prepare injections.

The present compound can be administered in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug.

For buccal administration, the compositions can be formulated as tablets or lozenges by conventional means.

For intranasal administration or administration by inhalation, the active compounds of the present disclosure are conveniently delivered in the form of a solution or suspension released from a pump spray container that is squeezed or pumped by the patient, or as an aerosol spray released from a pressurized container or nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules or cartridges (for example, made from gelatin) for use in an inhaler or insufflator can be formulated containing a powder mix of the present disclosure and a suitable powder base such as lactose or starch.

It is well known to those skilled in the art that the dosage of a drug depends on a variety of factors, including but not limited to, the following factors: activity of the specific compound, age, weight, general health, behavior, diet of the patient, administration time, administration route, excretion rate, drug combination and the like. In addition, the best treatment, such as treatment mode, daily dose of the compound of formula (I) or the type of pharmaceutically acceptable salt thereof can be verified by traditional therapeutic regimens.

Unless otherwise stated, the terms used in the specification and claims have the meanings described below.

“Alkyl” refers to a saturated aliphatic hydrocarbon group including C₁-C₂₀ straight chain and branched chain groups. Preferably an alkyl group is an alkyl having 1 to 12 (for example 1, 2, 3, 4, 5, 6, 7, 8, 9, 10, 11 or 12) carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. More preferably an alkyl group is a lower alkyl having 1 to 6 (for example 1, 2, 3, 4, 5 or 6) carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent group(s) is one or more, sometimes preferably one to five, and sometimes more preferably one to three, groups independently selected from alkyl, halogen, alkoxy, haloalkoxy, alkenyl, alkynyl, alkylthio, alkylamino, thiol, hydroxyl, nitro, cyano, amino, cycloalkyl, heterocyclyl, aryl, heteroaryl, cycloalkoxy, heterocylic, cycloalkylthio, heterocyclylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc. preferably C₂₋₂₀ alkenyl, more preferably C₂₋₁₂ alkenyl, and most preferably C₂₋₆ alkenyl. The alkenyl group can be substituted or unsubstituted. When substituted, the substituent group(s) is preferably one or more, sometimes preferably one to five, and sometimes more preferably one to three, group(s) independently selected from alkyl, halogen, alkoxy, haloalkoxy, alkenyl, alkynyl, alkylthio, alkylamino, thiol, hydroxyl, nitro, cyano, amino, cycloalkyl, heterocyclyl, aryl, heteroaryl, cycloalkoxy, heterocylic, cycloalkylthio, heterocyclylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₂₀ alkynyl, more preferably C₂₋₁₂ alkynyl, and most preferably C₂₋₆ alkynyl. The alkynyl group can be substituted or unsubstituted. When substituted, the substituent group(s) is preferably one or more, sometimes preferably one to five, and sometimes more preferably one to three, group(s) independently selected from alkyl, alkenyl, alkynyl, alkoxy, haloalkoxy, alkylthio, alkylamino, halogen, thiol, hydroxyl, nitro, cyano, cycloalkyl, heterocyclyl, aryl, heteroaryl, cycloalkoxy, heterocylic alkoxyl, cycloalkylthio and heterocyclylthio.

“Alkylene” refers to a saturated linear or branched aliphatic hydrocarbon group, wherein having 2 residues derived by removing two hydrogen atoms from the same carbon atom of the parent alkane or two different carbon atoms. The straight or branched chain group containing 1 to 20 carbon atoms, preferably has 1 to 12 (for example 1, 2, 3, 4, 5, 6, 7, 8, 9, 10, 11 or 12) carbon atoms, more preferably 1 to 6 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—), etc. The alkylene group can be substituted or unsubstituted. When substituted, the substituent group(s) is preferably one or more, sometimes preferably one to five, and sometimes more preferably one to three, group(s) independently selected from selected from alkyl, alkenyl, alkynyl, alkoxy, haloalkoxy, alkylthio, alkylamino, halogen, thiol, hydroxyl, nitro, cyano, cycloalkyl, heterocyclyl, aryl, heteroaryl, cycloalkoxy, heterocylic alkoxyl, cycloalkylthio and heterocyclylthio.

“Alkenylene” refers to an alkylene defined as above that has at least two carbon atoms and at least one carbon-carbon double bond, preferably C₂₋₂₀ alkenylene, more preferably C₂₋₁₂ alkenylene, and most preferably C₂₋₆ alkenylene. Non-limiting examples of alkenylene groups include, but are not limited to, —CH═CH—, —CH═CHCH₂—, —CH═CHCH₂CH₂—, —CH₂CH═CHCH₂— etc. The alkenylene group can be substituted or unsubstituted. When substituted, the substituent group(s) is preferably one or more, sometimes preferably one to five, and sometimes more preferably one to three, group(s) independently selected from selected from alkyl, alkenyl, alkynyl, alkoxy, haloalkoxy, alkylthio, alkylamino, halogen, thiol, hydroxyl, nitro, cyano, cycloalkyl, heterocyclyl, aryl, heteroaryl, cycloalkoxy, heterocylic alkoxyl, cycloalkylthio and heterocyclylthio.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, and most preferably 3 to 8 (for example 3, 4, 5, 6, 7 or 8) carbon atoms or 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more, preferably one to three, double bonds. Preferably a spiro cycloalkyl is 6 to 14 membered, and more preferably 7 to 10 (for example 7, 8, 9 or 10) membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following groups:

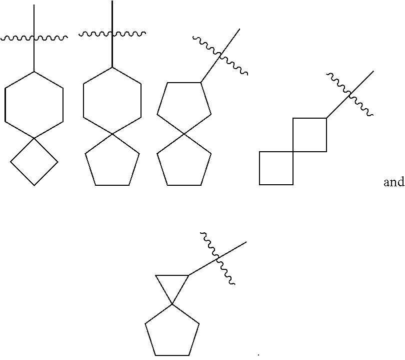

“Fused Cycloalkyl” refers to a 5 to 20 membered polycyclic hydrocarbon group, wherein each ring in the system shares an adjacent pair of carbon atoms with another ring, wherein one or more rings can contain one or more, preferably one to three, double bonds. Preferably, a fused cycloalkyl group is 6 to 14 membered, more preferably 7 to 10 (for example 7, 8, 9 or 10) membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 3-membered/4-membered, 3-membered/5-membered, 3-membered/6-membered, 4-membered/4-membered, 4-membered/5-membered, 4-membered/6-membered, 5-membered/4-membered, 5-membered/5-membered, 5-membered/6-membered, 6-membered/3-membered, 6-membered/4-membered, 6-membered/5-membered or 6-membered/6-membered bicyclic fused cycloalkyl. Representative examples of fused cycloalkyls include, but are not limited to, the following groups:

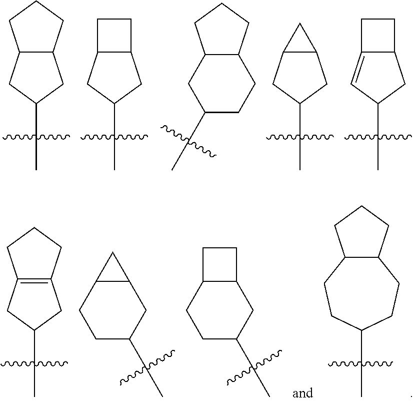

“Bridged Cycloalkyl” refers to a 5 to 20 membered polycyclic hydrocarbon group, wherein every two rings in the system share two disconnected carbon atoms. The rings can have one or more, preferably one to three, double bonds. Preferably, a bridged cycloalkyl is 6 to 14 membered, and more preferably 7 to 10 (for example 7, 8, 9 or 10) membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following groups:

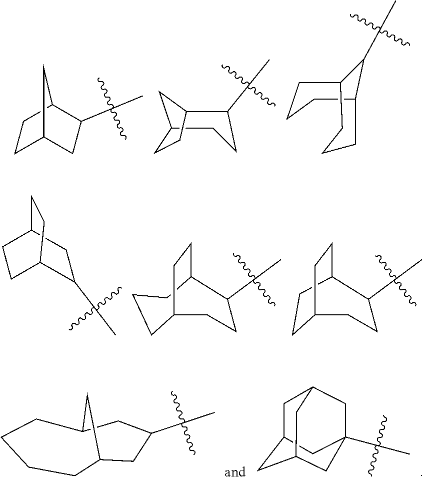

The cycloalkyl include the cycloalkyl said above which fused to the ring of an aryl, heteroaryl or heterocyclyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably one to five, and sometimes more preferably one to three, groups independently selected from alkyl, halogen, alkoxy, haloalkoxy, alkenyl, alkynyl, alkylthio, alkylamino, thiol, hydroxyl, nitro, cyano, amino, cycloalkyl, heterocyclyl, aryl, heteroaryl, cycloalkoxy, heterocylic, cycloalkylthio, heterocyclylthio and oxo group.

“Heterocyclyl” refers to a 3 to 20 membered saturated and/or partially unsaturated monocyclic or polycyclic hydrocarbon group having one or more, sometimes preferably one to five, and sometimes more preferably one to three, heteroatoms selected from N, O, S, S(O) and S(O)₂ as ring atoms, but excluding —O—O—, —O—S— or —S—S— in the ring, the remaining ring atoms being C. Preferably, heterocyclyl is a 3 to 12 (for example 3, 4, 5, 6, 7, 8, 9, 10, 11 or 12) membered having 1 to 4 (for example 1, 2, 3 or 4) heteroatoms; more preferably a 3 to 8 (for example 3, 4, 5, 6, 7 or 8) membered having 1 to 3 (for example 1, 2 or 3) heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more, sometimes preferably one to five, and sometimes more preferably one to three, heteroatoms selected from N, O, S, S(O) and S(O)₂ as ring atoms, the remaining ring atoms being C, wherein one or more rings can contain one or more, preferably one to three, double bonds. Preferably a spiro heterocyclyl is 6 to 14 membered, and more preferably 7 to 10 (for example 7, 8, 9 or 10) membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following groups:

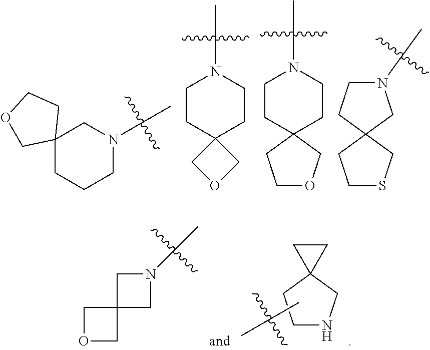

“Fused Heterocyclyl” refers to a 5 to 20 membered polycyclic heterocyclyl group, wherein each ring in the system shares an adjacent pair of carbon atoms with the other ring, wherein one or more rings can contain one or more, preferably one to three, double bonds, and wherein said rings have one or more heteroatoms selected from N, O, S, S(O) and S(O)₂ as ring atoms, the remaining ring atoms being C. Preferably a fused heterocyclyl is 6 to 14 membered, and more preferably 7 to 10 (for example 7, 8, 9 or 10) membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 3-membered/4-membered, 3-membered/5-membered, 3-membered/6-membered, 4-membered/4-membered, 4-membered/5-membered, 4-membered/6-membered, 5-membered/4-membered, 5-membered/5-membered, 5-membered/6-membered, 6-membered/3-membered, 6-membered/4-membered, 6-membered/5-membered or 6-membered/6-membered bicyclic fused heterocyclyl. Representative examples of fused heterocyclyl include, but are not limited to, the following groups:

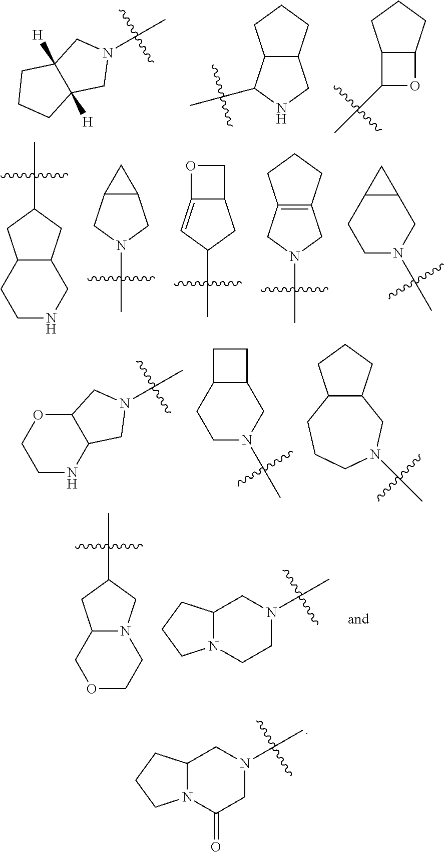

“Bridged Heterocyclyl” refers to a 5 to 14 membered polycyclic heterocyclyl group, wherein every two rings in the system share two disconnected atoms, the rings can have one or more, preferably one to three, double bonds, and the rings have one or more, sometimes preferably one to five, and sometimes more preferably one to three, heteroatoms selected from N, O, S, S(O) and S(O)₂ as ring atoms, the remaining ring atoms being C. Preferably a bridged heterocyclyl is 6 to 14 membered, and more preferably 7 to 10 (for example 7, 8, 9 or 10) membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following groups:

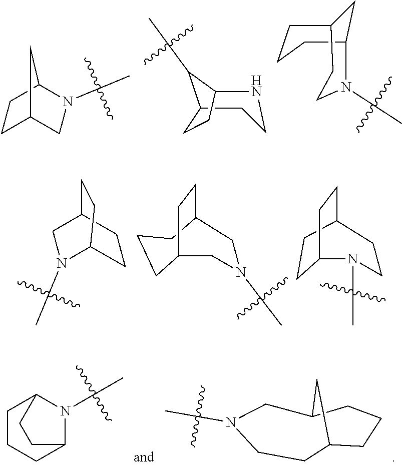

The ring of said heterocyclyl include the heterocyclyl said above which fused to the ring of an aryl, heteroaryl or cycloalkyl, wherein the ring bound to the parent structure is heterocyclyl. Representative examples include, but are not limited to the following groups:

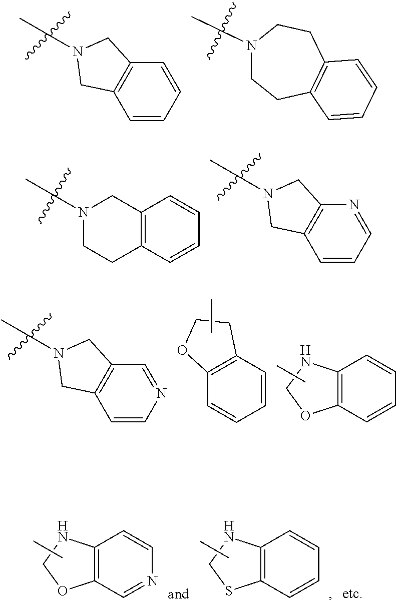

The heterocyclyl is optionally substituted or unsubstituted. When substituted, the substituent group(s) is preferably one or more, sometimes preferably one to five, and sometimes more preferably one to three, group(s) independently selected from alkyl, alkenyl, alkynyl, alkoxy, haloalkoxy, alkylthio, alkylamino, halogen, thiol, hydroxyl, nitro, cyano, cycloalkyl, heterocyclyl, aryl, heteroaryl, cycloalkoxy, heterocylic alkoxyl, cycloalkylthio and heterocyclylthio.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system.

Preferably aryl is 6 to 10 membered, such as phenyl and naphthyl, most preferably phenyl. The aryl include the aryl said above which fused to the ring of heteroaryl, heterocyclyl or cycloalkyl, wherein the ring bound to parent structure is aryl. Representative examples include, but are not limited to, the following groups:

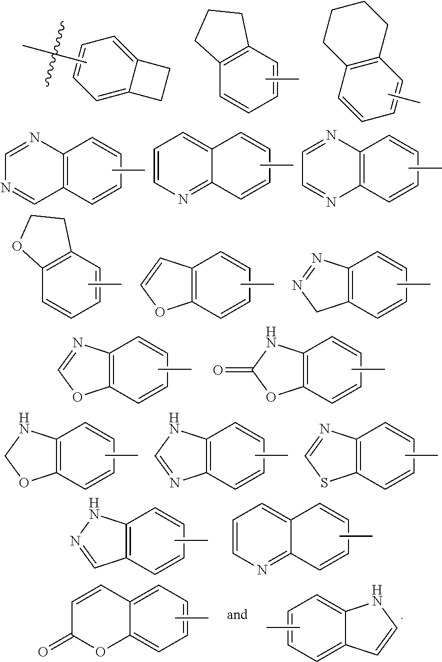

The aryl group can be substituted or unsubstituted. When substituted, the substituent group(s) is preferably one or more, sometimes preferably one to five, and sometimes more preferably one to three, groups independently selected from alkyl, alkenyl, alkynyl, alkoxy, haloalkoxy, alkylthio, alkylamino, halogen, thiol, hydroxyl, nitro, cyano, cycloalkyl, heterocyclyl, aryl, heteroaryl, cycloalkoxy, heterocylic alkoxyl, cycloalkylthio and heterocyclylthio.

“Heteroaryl” refers to an aryl system having 1 to 4 (for example 1, 2, 3 or 4) heteroatoms selected from O, S and N as ring atoms and having 5 to 14 annular atoms. Preferably a heteroaryl is 5- to 10- (for example 5, 6, 7, 8, 9 or 10) membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl include the heteroaryl said above which fused with the ring of an aryl, heterocyclyl or cycloalkyl, wherein the ring bound to parent structure is heteroaryl. Representative examples include, but are not limited to, the following groups:

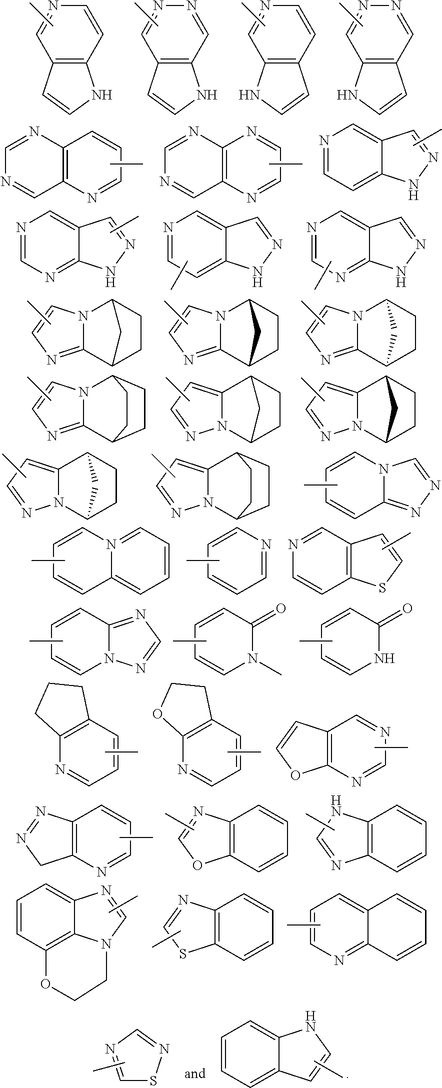

The heteroaryl group can be substituted or unsubstituted. When substituted, the substituent group(s) is preferably one or more, sometimes preferably one to five, and sometimes more preferably one to three, groups independently selected from alkyl, alkenyl, alkynyl, alkoxy, haloalkoxy, alkylthio, alkylamino, halogen, thiol, hydroxyl, nitro, cyano, cycloalkyl, heterocyclyl, aryl, heteroaryl, cycloalkoxy, heterocylic alkoxyl, cycloalkylthio and heterocyclylthio.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent is preferably one or more, sometimes preferably one to five, and sometimes more preferably one to three, groups independently selected from alkyl, alkenyl, alkynyl, alkoxy, haloalkoxy, alkylthio, alkylamino, halogen, thiol, hydroxyl, nitro, cyano, cycloalkyl, heterocyclyl, aryl, heteroaryl, cycloalkoxy, heterocylic alkoxyl, cycloalkylthio and heterocyclylthio.

The above-mentioned cycloalkyl, heterocyclyl, aryl and heteroaryl groups contain one monovalent residue derived from the removal of one hydrogen atom from the parent ring atom, or one divalent residue derived from the removal of two hydrogen atoms from the same or different ring atoms of the parent, namely “divalent cycloalkyl”, “divalent heterocyclyl group”, “arylene”, and “heteroarylene”.

The term “amino protecting group” is to keep the amino group unchanged when other parts of the molecule react, and to protect the amino group with a group that is easy to remove. Non-limiting examples include (trimethylsilyl)ethoxymethyl, tetrahydropyranyl, t-butoxycarbonyl, acetyl, benzyl, allyl, p-methoxybenzyl, and the like; sometimes preferably tetrahydropyranyl; and sometimes further preferably tetrahydropyran-2-yl. These groups may be optionally substituted with 1-3 substituents selected from halogen, alkoxy or nitro.

“Bond” refers to a covalent bond using a sign of “—”.

“Hydroxyalkyl” refers to an alkyl group substituted by a hydroxyl group, wherein alkyl is as defined above.

“Hydroxyl” refers to an —OH group.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(═O)OH group.

“Haloalkyl” refers to an alkyl group substituted by a halogen group, wherein alkyl is as defined above.

“haloalkoxy” refers to a haloalkyl-O—, wherein alkyl is as defined above.

“thiol” refers to a —SH group.

“alkylthio” refers to a alkyl-S— group, wherein alkyl is as defined above.

“haloalkylthio” refers to a haloalkyl-S— group, wherein haloalkyl is as defined above.

“cycloalkoxy” refers to a cycloalkyl-O—, wherein cycloalkyl is as defined above.

“heterocyclyloxy” refers to a heterocyclyl-O—, wherein heterocyclyl is as defined above.

“cycloalkylthio” refers to a cycloalkyl-S—, wherein cycloalkyl is as defined above.

“heterocyclylthio” refers to a heterocyclyl-S—, wherein heterocyclyl is as defined above.

THP refers to

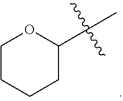 [SMILES: *C1CCCCO1]

Boc refers to

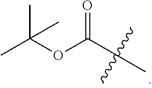 [SMILES: *C(=O)OC(C)(C)C]

OTf refers to

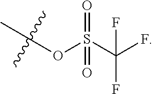 [SMILES: *OS(=O)(=O)C(F)(F)F]

“Alkoxycarbonyl” refers to a —C(═O)O(alkyl) or —C(═O)O (cycloalkyl) group, wherein the alkyl and cycloalkyl are defined as above.

“Optional” or “optionally” means that the event or circumstance described subsequently can, but need not, occur, and the description includes the instances in which the event or circumstance may or may not occur. For example, “the heterocyclyl group optionally substituted by an alkyl” means that an alkyl group can be, but need not be, present, and the description includes the case of the heterocyclyl group being substituted with an alkyl and the heterocyclyl group being not substituted with an alkyl.

“Substituted” refers to one or more hydrogen atoms in the group, preferably up to 5, more preferably 1 to 3 hydrogen atoms, independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present disclosure or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the disclosure, such salts being safe and effective when used in a mammal and have corresponding biological activity.

The compounds of the present disclosure may also contain unnatural proportions of atomic isotopes at one or more of the atoms that constitute such compounds. Unnatural propoliions of an isotope may be defined as ranging from the amount found in nature to an amount consisting of 100% of the atom in question. For example' the compounds may incorporate radioactive isotopes, such as for example tritium (3H), iodine-125 (¹²⁵I) or carbon-14 (¹⁴C), or non-radioactive isotopes, such as deuterium (D) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents.

### Synthesis Method

In order to complete the purpose of the disclosure, the present disclosure applies, but is not limited to, the following technical solution:

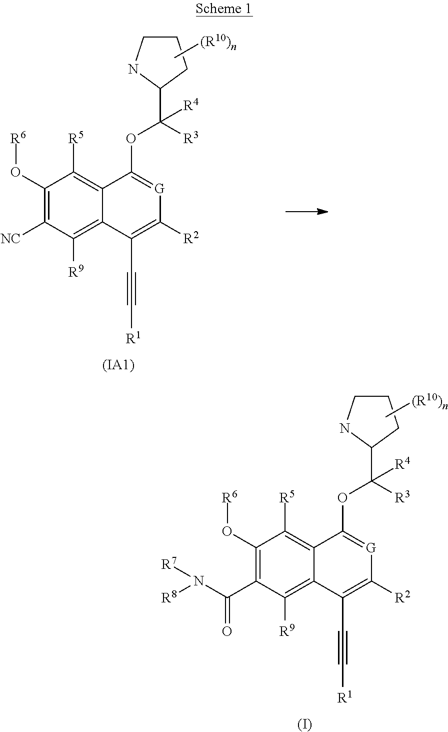

A preparation process of a compound of formula (I), or a tautomer, mesomer, racemate, enantiomer, diastereomer, or mixture thereof, or a pharmaceutically acceptable salt thereof comprising a step of:

the compound of formula (IA1) is subjected to a hydration reaction under acidic or alkaline conditions in the presence of catalyst, to obtain the compound of formula (I), wherein:

R⁷ is hydrogen;

R⁸ is hydrogen;

G, R¹ to R⁶, R⁹, R¹⁰ and n are each as defined in formula (I).

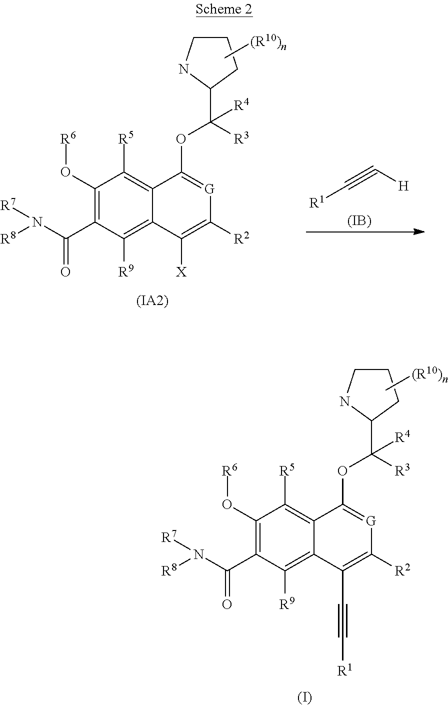

A preparation process of a compound of formula (I), or a tautomer, mesomer, racemate, enantiomer, diastereomer, or mixture thereof, or a pharmaceutically acceptable salt thereof, thereof comprising a step of:

formula (IA2) and formula (IB) undergo Sonogashira coupling reaction under alkaline conditions in the presence of a metal catalyst, to obtain the compound of formula (I), wherein:

X is halogen or OTf;

G is N or CR^{g}; preferably is CR^{g};

R^{g}, R¹ to R¹⁰ and n are each as defined in formula (I).

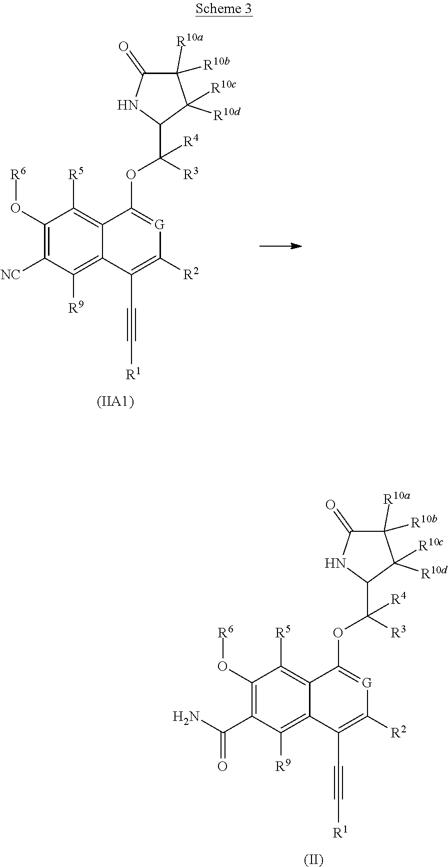

A preparation process of a compound of formula (II), or a tautomer, mesomer, racemate, enantiomer, diastereomer, or mixture thereof, or a pharmaceutically acceptable salt thereof comprising a step of:

the compound of formula (IIA1) is subjected to hydration reaction under acidic or alkaline conditions in the presence of catalyst, to obtain the compound of formula (II), wherein:

G, R¹-R⁶, R⁹, R^{10a}, R^{10b}, R^{10c} and R^{10d} a are each as defined in formula (II).

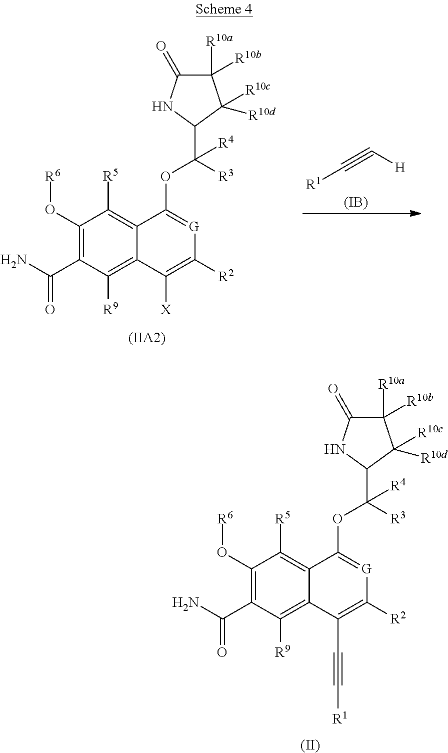

A preparation process of a compound of formula (II), or a tautomer, mesomer, racemate, enantiomer, diastereomer, or mixture thereof, or a pharmaceutically acceptable salt thereof, thereof comprising a step of:

formula (IIA2) and formula (IB) undergp Sonogashira coupling reaction under alkaline conditions in the presence of a metal catalyst, to obtain the compound of formula (II), wherein:

X is Cl, Br, I, or OTf;

G is N or CR^{g}; preferably is CR^{g};

R^{g}, R¹-R⁶, R⁹, R^{10a}, R^{10b}, R^{10c} and R^{10d} a are each as defined in formula (II).

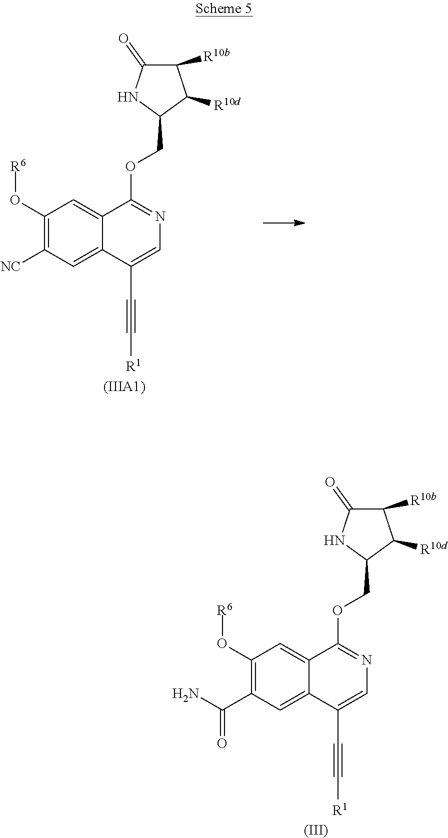

A preparation process of a compound of formula (III), or a tautomer, mesomer, racemate, enantiomer, diastereomer, or mixture thereof, or a pharmaceutically acceptable salt thereof comprising a step of:

the compound of formula (IIIA1) is subjected to hydration reaction under acidic or alkaline conditions in the presence of catalyst, to obtain the compound of formula (III), wherein:

R¹, R⁶, R^{10b} and R^{10d} are each as defined in formula (III).

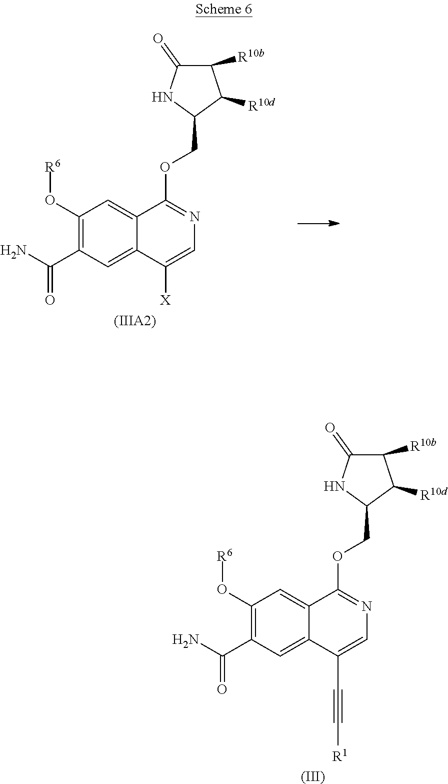

A preparation process of a compound of formula (III), or a tautomer, mesomer, racemate, enantiomer, diastereomer, or mixture thereof, or a pharmaceutically acceptable salt thereof, thereof comprising a step of:

formula (IIIA2) and formula (IB) undergo Sonogashira coupling reaction under alkaline conditions in the presence of a metal catalyst, to obtain the compound of formula (III), wherein:

X is halogen or OTf;

R¹, R⁶, R^{10b} and R^{10d} are each as defined in formula (III).

The acids that provide acidic conditions include, but are not limited to, acetic acid, pyridine hydrobromide, trifluoroacetic acid, formic acid, hydrochloric acid, sulfuric acid, p-toluenesulfonic acid, p-toluenesulfonic acid monohydrate and methanesulfonic acid; preferably hydrochloric acid or p-toluenesulfonic acid monohydrate.

The reagents that provide alkaline conditions include organic bases and inorganic bases, wherein the organic base includes, but is not limited to, triethylamine, diethylamine, N,N-disopropylethylamine, n-butyllithium, lithium diisopropylamide, potassium acetate, sodium tert-butoxide, N-(3-Dimethylaminopropyl)-N′-ethylcarbodiimide hydrochloride (EDCI), potassium bis(trimethylsilyl)amide (KHMDS) and potassium tert-butoxide, and wherein the inorganic base includes, but is not limited to, magnesium chloride, sodium hydride, potassium phosphate, sodium carbonate, potassium carbonate, cesium carbonate and sodium hydroxide; preferably potassium carbonate, cesium carbonate and KHMDS.

The catalysts for the hydrolysis reaction include, but are not limited to, MnO₂, H₂O₂, NiO, CeO₂ and ruthenium hydroxide; preferably is H₂O₂.

The metal catalysts for the Sonogashira coupling reaction include palladium catalysts and copper catalysts, wherein the palladium catalysts include but is not limited to Pd(PPh₃)₄, Pd(PPh₃)₂Cl₂, Pd(dppe)Cl₂, Pd(dppp)Cl₂ and Pd(dppf)Cl₂; preferably is Pd(PPh₃)₂Cl₂. The copper catalysts include but is not limited to CuI, CuCl, CuBr, preferably is CuI.

The reaction is preferably conducted in solvent, wherein suitable solvents include, but are not limited to, acetic acid, methanol, ethanol, ether, toluene, tetrahydrofuran, dichloromethane, dimethylsulfoxide, 1,4-dioxane, water, N,N-dimethylformamide and the mixture thereof.

### Methods of Preparation

### General Synthetic Schemes

The compounds of the present invention can be prepared in several ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. The reactions and techniques described in this section are performed in solvents appropriate to the reagents and materials employed and are suitable for the transformations being affected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene et al. (Protective Groups in Organic Synthesis, Third Edition, Wiley and Sons (1999)). Compounds of the Formula (I) can be prepared according to the methods outlined in the following schemes.

In Scheme 1, the ether intermediate 2 can be prepared from chloride 1 by a SnAr reaction in the presence of base such as Potassium bis(trimethylsilyl)amide.

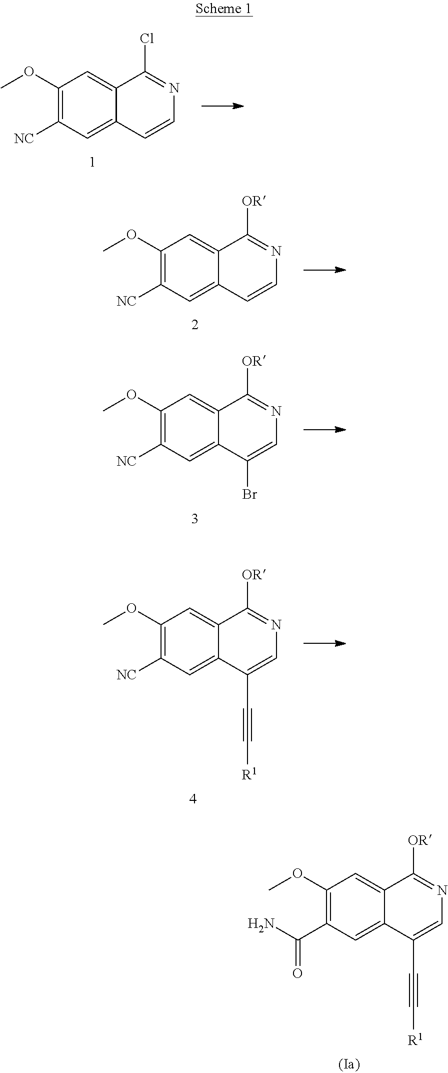

Treatment of intermediate 2 with NBS results in bromination product of 4-bromoisoquinoline intermediate 3. Subsequent Sonogashira coupling of bromide 3 can afford acetylene compound 4. Treatment of 4 with potassium carbonate and hydrogen peroxide in solvents such as DMSO affords compound of Formula (Ia).

Scheme 2 illustrates a method for the preparation of compounds of Formula (Ib). A Fisher esterification of naphthoic acid 5 could produce methyl ester intermediate 6. The distal hydroxyl group in compound 6 may be selectively protected as silyl ether to afford intermediate 7. Alkylation of the proximal hydroxyl group by Williamson ether synthesis method, or by Mitsunobu reaction with an alcohol ROH in the presence of triphenylphosphine and an azodicarboxylate ester could produce intermediate 8, which is then deprotected and the resulting hydroxyl group is alkylated by Williamson ether synthesis or Mitsunobu reaction to produce intermediate 9. The ester group in intermediate 9 is transferred to its corresponding carboxamide by directly reacting with ammonia to afford compound of Formula (Ib).

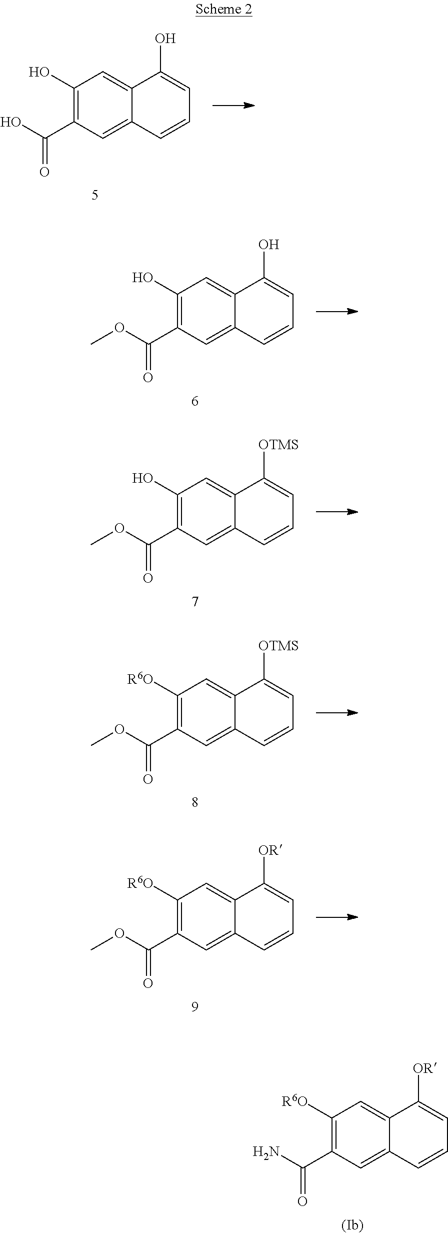

Scheme 3 illustrates a method for the preparation of additional compounds of Formula (Ic). In this method, Compound 1 is de-alkylated to remove the methyl group to afford a compound of Formula 10. De-alkylation may be affected by standard methods known to one skilled in the art, for example by the use of anhydrous aluminum chloride or boron tribromide in a solvent such as DCM. Subsequent alkylation of the OH group may be effected to install a new R⁶ group to afford compound 11, using methods known to one skilled in the art, for example by treatment with an alcohol R⁶—OH in the presence of triphenylphosphine and an azodicarboxylate ester (“Mitsunobu reaction”) in a suitable solvent such as THF, or by treatment with an alkylating agent such as an alkyl chloride, bromide, or iodide (R⁶—Cl, R⁶—Br, or R⁶—I) and a base such as K₂CO₃ in a suitable solvent such as THE or DMF. the ether intermediate 12 can be prepared from chloride 11 by a SnAr reaction in the presence of base such as Potassium bis(trimethylsilyl)amide. Treatment of intermediate 12 with NBS results in bromination product of 4-bromoisoquinoline intermediate 13. Subsequent Sonogashira coupling of bromide 3 can afford acetylene compound 14. Treatment of 14 with potassium carbonate and hydrogen peroxide in solvents such as DMSO affords compound of Formula (Ic).

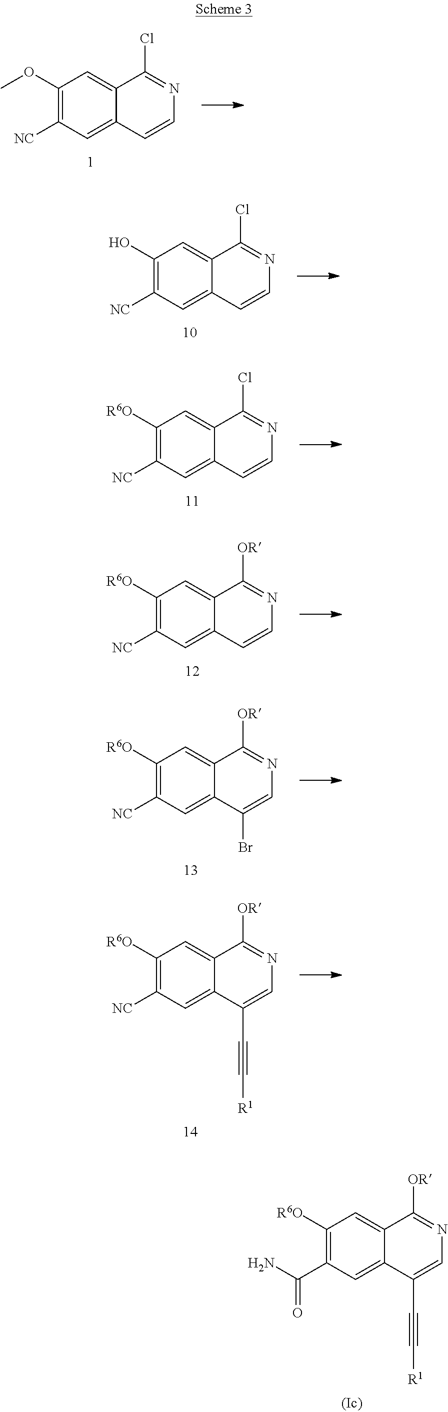

R′ in Scheme 1 to Scheme 3 corresponds to

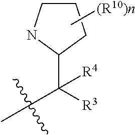

### in Formula I, or

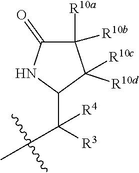

### in Formula II, or

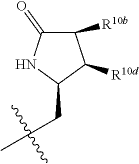

in formula III above.

### Experimental Procedures and Working Examples

The following illustrate the synthesis of various compounds of the present invention. Additional compounds within the scope of this invention may be prepared using the methods illustrated in these Examples, either alone or in combination with techniques generally known in the art. It will be understood that the intermediate compounds of the invention depicted above are not limited to the particular enantiomer shown, but also include all stereoisomers and mixtures thereof. It will also be understood that compounds of Formula (I) can include intermediates of compounds of Formula (I). All patent or non-patent references are incorporated herein by references in their entirety without admission of them as prior art.

### Experimental Procedures

Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Products were generally dried under vacuum before being carried on to further reactions or submitted for biological testing. Mass spectrometry data is reported from either liquid chromatography-mass spectrometry (LCMS), atmospheric pressure chemical ionization (APCI) or gas chromatography-mass spectrometry (GCMS) instrumentation. Chemical shifts for nuclear magnetic resonance (NMR) data are expressed in parts per million (ppm, S) referenced to residual peaks from the deuterated solvents employed.

For syntheses referencing procedures in other Examples or Methods, reaction conditions (length of reaction and temperature) may vary. In general, reactions were followed by thin layer chromatography and/or liquid chromatography-mass spectrometry, and subjected to work-up when appropriate. It will be recognized by one skilled in the art that purifications may vary between experiments: in general, sorbents, solvents and the solvent ratios used for eluants/gradients were chosen to provide appropriate Rfs or retention times. It will also be recognized by one skilled in the art that HPLC purifications may be effected in a variety of ways, including the use of normal stationary phases, reverse stationary phases, chiral stationary phases, and supercritical eluants. The appropriate choices of conditions for chromatographic and HPLC purifications will be discerned by one skilled in the art. The following Preparations describe the preparation of certain intermediates used in the Methods and Examples that follow.

### General Information

The structure of the compound was determined by nuclear magnetic resonance (NMR)/mass spectrometry (MS). All NMR spectra were recorded on Bruker AVANCE II-400 MHz spectrometer at STP unless otherwise indicated. NMR chemical shifts were reported in ppm and referenced to TMS (δ=0.00 ppm, ¹H NMR) or the residual solvent peak. Deuterated solvents such as DMSO-d⁶, CDCl₃ and CD₃OD were used in general NMR test.

LC/MS (ESI) analyses were performed on a Shimadzu LCMS2020 equipped with a Sunfire C18 (5 μm 50×4.6 mm) column, Waters UPLC-QDa equipped with an ACQUITY UPLC® BEH (2.1*50 mm 1.7 um) column, Agilent Agilent6120 equipped with a Xbridge C18 (5 μm 50×4.6 mm) column.

HPLC analyses were performed on an Agilent 1200DAD equipped with a Sunfire C18 (5 μm 150×4.6 mm) column and Shimadzu UFLC equipped with an Xbridge C18 (5 μm 150×4.6 mm) column.

Chiral HPLC analyses were performed on a Waters-UPC² instrument.

Analytical thin layer chromatography (TLC) was performed using 0.2 mm-0.25 mm silica gel plates (purchased from Huanghai Yantai, Xinnuo and Shandong Rushan). Prepared thin layer chromatography (PLC) was performed on using 0.4 mm-0.5 mm silica gel plates. Column chromatography was performed using silica gel 100-200 mesh or 200-300 mesh (purchased from YuCheng Chemical Shanghai, Co., Ltd) as the solid support.

Pre-HPLC was performed on a Waters 2767 equipped with a Sunfire Pre C18 (10 μm 19×250 mm) column and Waters 2767-QDa equipped with an Xbridge Pre C18 (10 μm 19×250 mm) column instrument.

Pre-SFC was performed on a Waters-SFC80 equipped with Daciel AD/OD/OJ/IC/IA/ID (10 μm 20×250 mm) column instrument.

CombiFlash was performed on an Agela Technologies.

The source should be indicated if the starting material was purchased and used as received. For example: ABCR GmbH & Co. KG, Acros Organics, Aldrich Chemical Company, Accela ChemBio Inc, etc.

All reactions were carried out under an atmosphere of nitrogen unless otherwise indicated. High pressure hydrogenation was performed on GSH-1/12.5, GSH-2/12.5, GSH-5/12.5, GSH-20/12.5 autoclave.

Microwave reaction was performed on a Monowave 300 and Initiateor+.

The ratio of eluent system should be recorded clearly when using TLC or purification with silica gel chromatography column. The addition of small amounts of TEA or acetic acid and other basic or acidic reagents also needs to be clearly labeled.

The following Preparations, Methods and Examples are intended to illustrate particular embodiments of the invention and preparations thereto and are not intended to limit the specification, including the claims, in any manner. Unless noted otherwise, all reactants were obtained commercially.

Unless indicated otherwise, the following abbreviations have the indicated meanings:

APCI—atmospheric pressure chemical ionization

br.—broad peaks

° C.—degree Celsius

CDCl₃—deuterated chloroform

CD₃OD—deuterated methanol

d—doublet peak

dd—double doublet peak

D₂O—deuterium oxide

dmso-d₆—perdeuterated dimethyl sulfoxide

dt—double triplet peak

g—gram(s)

H (e.g., 1H, 2H)—hydrogen(s)

hr—hour(s)

LC—liquid chromatography

m—multiplet

M—molarity

mg—milligram(s)

MHz—megahertz

min—minute(s)

mL—milliliter(s)

mmol—millimole(s)

mp—melting point

MS—mass spectrum

NMR—nuclear magnetic resonance

pH—negative logarithm of hydronium ion concentration

psi—pounds per square inch

q—quartet peak

s—singlet peak

t—triplet peak

td—triple doublet peak

μL-microliter.

### Preparations

### Example 1: tert-butyl (4-(6-carbamoyl-1-(((2S,3S,4S)-3-ethyl-4-fluoro-5-oxopyrrolidin-2-yl)methoxy)-7-methoxyisoquinolin-4-yl)-2-methylbut-3-yn-2-yl)carbamate 1

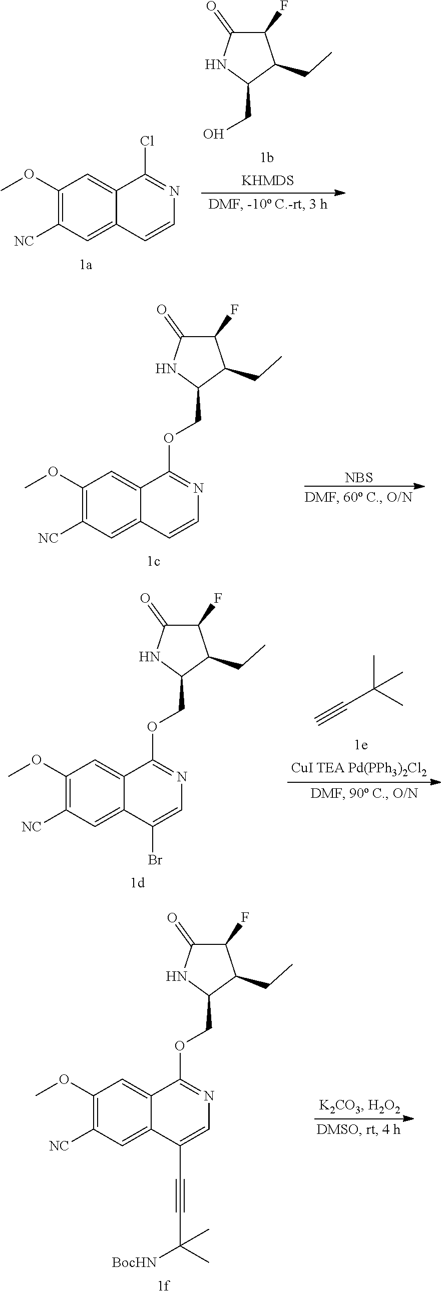

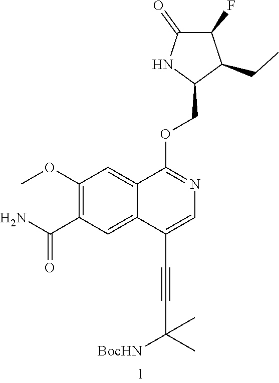 [SMILES: CC[C@@H]1[C@H](F)C(=O)N[C@@H]1COc1ncc(C#CC(C)(C)NC(=O)OC(C)(C)C)c2cc(C(N)=O)c(OC)cc12]

### Step 1. Synthesis of 1-(((2S,3S,4S)-3-ethyl-4-fluoro-5-oxopyrrolidin-2-yl)methoxy)-7-methoxyisoquinoline-6-carbonitrile 1c

A mixture of 1-chloro-7-methoxyisoquinoline-6-carbonitrile 1a (436 mg, 2.0 mmol, Pharmablock) and (3S,4S,5S)-4-ethyl-3-fluoro-5-(hydroxymethyl)pyrrolidin-2-one 1b (354 mg, 2.2 mmol, Pharmablock) in DMF (10 mL, Aldrich) was treated with KHMDS (1 M in THF, 4.4 mL, Aldrich) at −10° C. Upon completion of the KHMDS addition, the cooling bath was removed and the mixture was stirred at rt for 3 h. The reaction mixture was then poured into a mixture of 10% (w/v) NaH₂PO₄ and DCM with vigorous stirring. The DCM was separated, washed with water, brine, dried over Na₂SO₄, filtered, and concentrated. The residue was purified by chromatography to afford 360 mg (Yield: 52%) of title compound 1c.

### Step 2. Synthesis of 4-bromo-1-(((2S,3S,4S)-3-ethyl-4-fluoro-5-oxopyrrolidin-2-yl)methoxy)-7-methoxyisoquinoline-6-carbonitrile 1d

To a stirred solution of 1c (360 mg, 1.05 mmol) in DMF (5 mL, Aldrich) at rt was added NBS (280 mg, 1.57 mmol, Aldrich) and the resulting mixture was stirred at 60° C. overnight. After completion of the reaction, the mixture was poured into water and extracted with DCM. The DCM was separated, washed with water, brine, dried over Na₂SO₄, filtered, and concentrated. The residue was purified by chromatography to afford 350 mg (Yield: 79%) of title compound 1d.

### Step 3. tert-butyl (4-(6-cyano-1-(((2S,3S,4S)-3-ethyl-4-fluoro-5-oxopyrrolidin-2-yl)methoxy)-7-methoxyisoquinolin-4-yl)-2-methylbut-3-yn-2-yl)carbamate 1f

A mixture of 1d (88 mg, 0.21 mmol), tert-butyl (2-methylbut-3-yn-2-yl)carbamate 1e (76 mg, 0.42 mmol, Aldrich), Pd(PPh₃)₂Cl₂ (15 mg, 0.021 mmol, Aldrich), CuI (8 mg, 0.042 mmol, Aldrich), TEA (1 mL, Aldrich) and DMF (3 mL, Aldrich) was flushed with N2 for 3 mins and then the mixture was stirred at 90° C. overnight. After completion of the reaction, the mixture was poured into water and extracted with DCM. The DCM was separated, washed with water, brine, dried over Na₂SO₄, filtered, and concentrated. The residue was purified by chromatography to afford 84 mg (Yield: 80%) of title compound 1f.

### Step 4. tert-butyl (4-(6-carbamoyl-1-(((2S,3S,4S)-3-ethyl-4-fluoro-5-oxopyrrolidin-2-yl)methoxy)-7-methoxyisoquinolin-4-yl)-2-methylbut-3-yn-2-yl)carbamate 1

A mixture of 1f (84 mg, 0.16 mmol) and K₂CO₃ (88 mg, 0.64 mmol, Aldrich) in DMSO (3 mL, Aldrich) was treated with H₂O₂ (30%, 163 uL, 1.6 mmol, Aldrich) added dropwise at rt. The mixture was stirred at rt for 4 h. After completion of the reaction, the mixture was poured into water and extracted with DCM. The DCM was separated, washed with water, brine, dried over Na₂SO₄, filtered, and concentrated. The residue was purified by chromatography to afford 55 mg (Yield: 65%) of the title compound 1. ¹H NMR (400 MHz, CDCl₃, ppm): δ 8.7 (s, 1H), 8.1 (s, 1H), 8.0 (s, 1H), 7.6 (brs, 1H), 7.4 (s, 1H), 6.4 (brs, 1H), 5.4 (brs, 1H), 5.0-4.8 (m, 1H), 4.8-4.6 (m, 1H), 4.5-4.4 (m, 1H), 4.3-4.2 (m, 1H), 3.9 (s, 3H), 2.7-2.4 (m, 1H), 1.8 (s, 6H), 1.8-1.6 (m, 2H), 1.5 (s, 9H), 1.2-1.0 (m, 3H). MS m/z (ESI): 543 [M+1].

### Example 2: 4-(3-amino-3-methylbut-1-yn-1-yl)-1-(((2S,3S,4S)-3-ethyl-4-fluoro-5-oxopyrrolidin-2-yl)methoxy)-7-methoxyisoquinoline-6-carboxamide 2

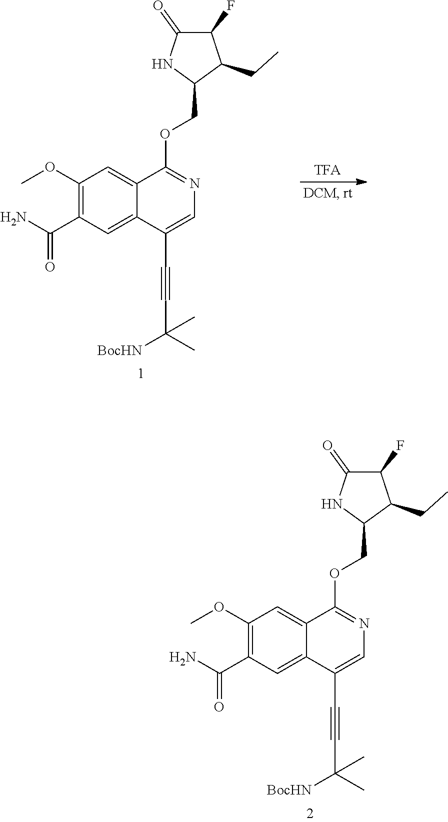

### Step 1. 4-(3-amino-3-methylbut-1-yn-1-yl)-1-(((2S,3S,4S)-3-ethyl-4-fluoro-5-oxopyrrolidin-2-yl)methoxy)-7-methoxyisoquinoline-6-carboxamide 2

To a stirred solution of 1 (51 mg, 0.094 mmol) in DCM (3 mL, Aldrich) at rt was added TFA (2 mL, Aldrich) and the resulting mixture was stirred at rt for 3 h. After completion of the reaction, the mixture was poured into water and extracted with DCM. The DCM was separated, washed with water, brine, dried over Na₂SO₄, filtered, and concentrated. The residue was purified by chromatography to afford 35 mg (Yield: 83%) of the title compound 2. ¹H NMR (400 MHz, CDCl₃, ppm): δ 8.6 (s, 1H), 8.3 (brs, 1H), 7.9 (s, 1H), 7.6 (brs, 1H), 7.4 (s, 1H), 6.5 (brs, 1H), 5.0-4.6 (m, 2H), 4.5-4.3 (m, 1H), 4.2-4.1 (m, 1H), 3.9 (s, 3H), 2.5-2.3 (m, 1H), 2.3 (brs, 2H), 1.8-1.4 (m, 2H), 1.5 (s, 6H), 1.2-1.0 (m, 3H). MS m/z (ESI): 443 [M+1].

### Example 3: 1-(((2S,3S,4S)-3-ethyl-4-fluoro-5-oxopyrrolidin-2-yl)methoxy)-4-(3-hydroxyl-3-methylbut-1-yn-1-yl)-7-methoxyisoquinoline-6-carboxamide 3

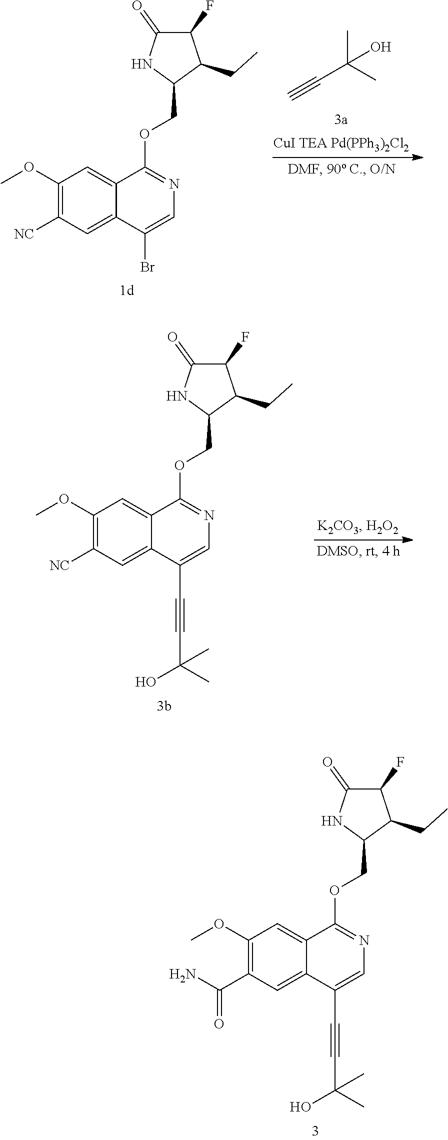

### Step 1. 1-(((2S,3S,4S)-3-ethyl-4-fluoro-5-oxopyrrolidin-2-yl)methoxy)-4-(3-hydroxyl-3-methylbut-1-yn-1-yl)-7-methoxyisoquinoline-6-carbonitrile 3b

A mixture of 1d (66 mg, 0.156 mmol), 2-methylbut-3-yn-2-ol 3a (1 ml, Aldrich), Pd(PPh₃)₂Cl₂ (11 mg, 0.016 mmol, Aldrich), CuI (6 mg, 0.031 mmol, Aldrich), TEA (1 mL, Aldrich) and DMF (3 mL, Aldrich) was flushed with N2 for 3 mins and then the mixture was stirred at 90° C. overnight. After completion of the reaction, the mixture was poured into water and extracted with DCM. The DCM was separated, washed with water, brine, dried over Na₂SO₄, filtered, and concentrated. The residue was purified by chromatography to afford 30 mg (Yield: 50%) of title compound 3b.

### Step 2. 1-(((2S,3S,4S)-3-ethyl-4-fluoro-5-oxopyrrolidin-2-yl)methoxy)-4-(3-hydroxyl-3-methylbut-1-yn-1-yl)-7-methoxyisoquinoline-6-carboxamide 3

A mixture of 3b (30 mg, 0.07 mmol) and K₂CO₃ (39 mg, 0.28 mmol, Aldrich) in DMSO (3 mL, Aldrich) was treated with H₂O₂ (30%, 71 uL, 0.7 mmol, Aldrich) added dropwise at rt. The mixture was stirred at rt for 4 h. After completion of the reaction, the mixture was poured into water and extracted with DCM. The DCM was separated, washed with water, brine, dried over Na₂SO₄, filtered, and concentrated. The residue was purified by chromatography to afford 21 mg (Yield: 68%) of the title compound 3. ¹H NMR (400 MHz, CDCl₃, ppm): δ 8.9 (s, 1H), 8.5 (s, 1H), 7.8 (s, 1H), 7.6 (brs, 1H), 7.2 (s, 1H), 6.3 (brs, 1H), 5.0-4.7 (m, 2H), 4.5-4.3 (m, 2H), 4.2-4.1 (m, 1H), 3.8 (s, 3H), 2.7-2.4 (m, 1H), 1.7 (s, 3H), 1.6 (s, 3H), 1.6-1.4 (m, 2H), 1.0-0.9 (m, 3H). MS m/z (ESI): 444 [M+1].

### Example 4: 3-(6-carbamoyl-1-(((2S,3S,4S)-3-ethyl-4-fluoro-5-oxopyrrolidin-2-yl)methoxy)-7-methoxyisoquinolin-4-yl)propiolic acid 4

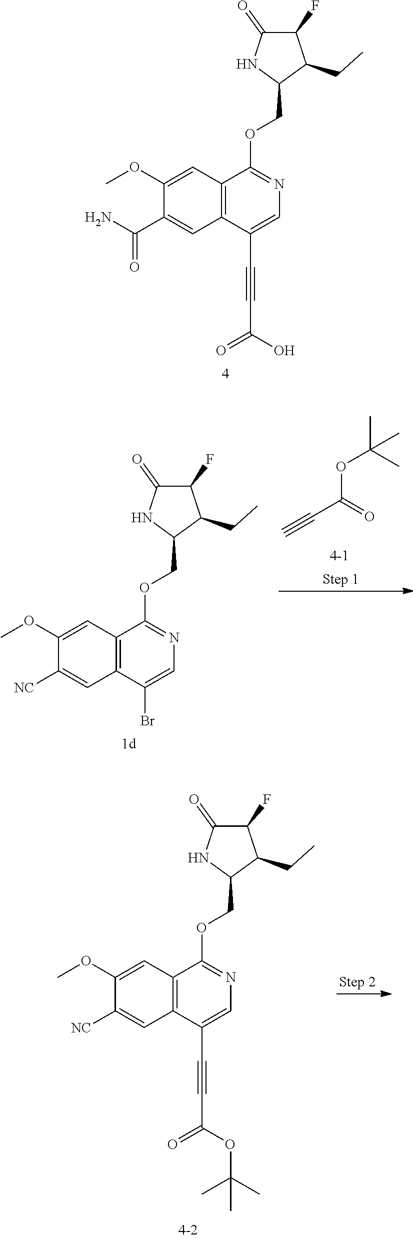

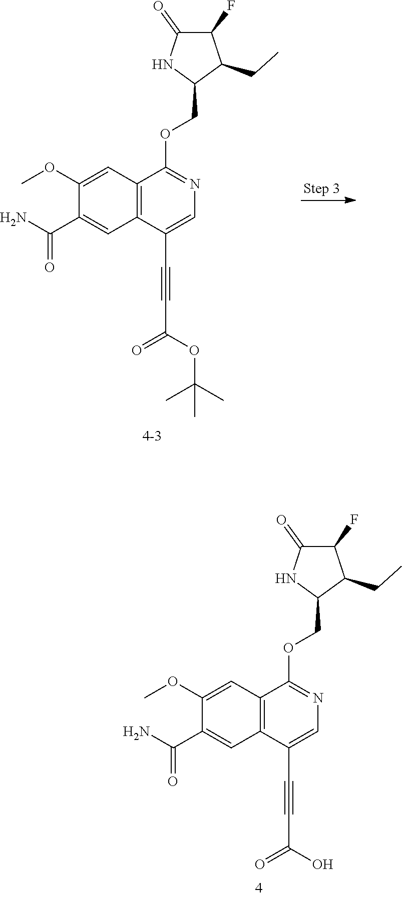

### Step 1. Synthesis of tert-butyl 3-(6-cyano-1-(((2S,3S,4S)-3-ethyl-4-fluoro-5-oxopyrrolidin-2-yl)methoxy)-7-methoxyisoquinolin-4-yl)propiolate 4-2

The mixture of 1d (100 mg, 236.83 umol), tert-butyl propiolate 4-1 (29.88 mg, 236.83 umol, Bidepharm), Pd(PPh₃)Cl₂ (25 mg, 118.41 umol), CuI (22.55 mg, 118.41 umol) and TEA (95.86 mg, 947.31 umol) in DMF (3 mL) was stirred at 90° C. for 15 hours under N2. The mixture was diluted with water and then extracted with EA (100 mL). The organic solution was dried and concentrated. The residue was purified by SGC (DCM:MeOH=50:1) to give the title compound 4-2 (50 mg, 106.95 umol, 45.16% yield). MS m/z (ESI): 412.1 [M−56]⁺.

### Step 2. Synthesis of tert-butyl 3-(6-carbamoyl-1-(((2S,3S,4S)-3-ethyl-4-fluoro-5-oxopyrrolidin-2-yl)methoxy)-7-methoxyisoquinolin-4-yl)propiolate4-3

The mixture of 4-2 (50 mg, 106.95 umol) and K₂CO₃ (100 mg, 320.86 umol) in H₂O₂ (30%) (0.2 mL) and DMSO (2 mL) was stirred at rt for 3 hours. The mixture was diluted with water and then extracted with EA (50 mL). The organic solution was dried and concentrated. The residue was purified by SGC (DCM:MeOH=30:1) to give the title compound 4-3 (30 mg, 61.79 umol, 57.77% yield). MS m/z (ESI): 430.1 [M−56]⁺.

### Step 3. Synthesis of 3-(6-carbamoyl-1-(((2S,3S,4S)-3-ethyl-4-fluoro-5-oxopyrrolidin-2-yl)methoxy)-7-methoxyisoquinolin-4-yl)propiolic acid 4

The solution of 4-3 (30 mg, 64.17 umol) in DCM (2 mL) and TFA (0.4 mL) was stirred rt for 2 hours. The mixture was diluted with water and adjusted pH=7, then extracted with EA (30 mL). The organic solution was dried and concentrated. The residue was purified by prep-HPLC to give the title compound 4 (5 mg, 11.64 umol, 18.15% yield).

¹H NMR (400 MHz, DMSO-d₆, ppm) δ 8.90 (s, 1H), 8.36 (s, 1H), 8.25 (s, 1H), 7.96 (s, 1H), 7.85 (s, 1H), 7.81 (s, 1H), 4.91 (dd, 1H), 4.60 (dd, 1H), 4.33 (dd, 1H), 4.11 (brs, 1H), 3.99 (s, 3H), 2.70-2.54 (m, 1H), 1.59-1.57 (m, 2H), 1.02 (t, 3H). MS m/z (ESI): 859.7 [2M+H]⁺.

### Example 5: 4-(3-(dimethylamino)prop-1-yn-1-yl)-1-(((2S,3S,4S)-3-ethyl-4-fluoro-5-oxopyrrolidin-2-yl)methoxy)-7-methoxyisoquinoline-6-carboxamide 5

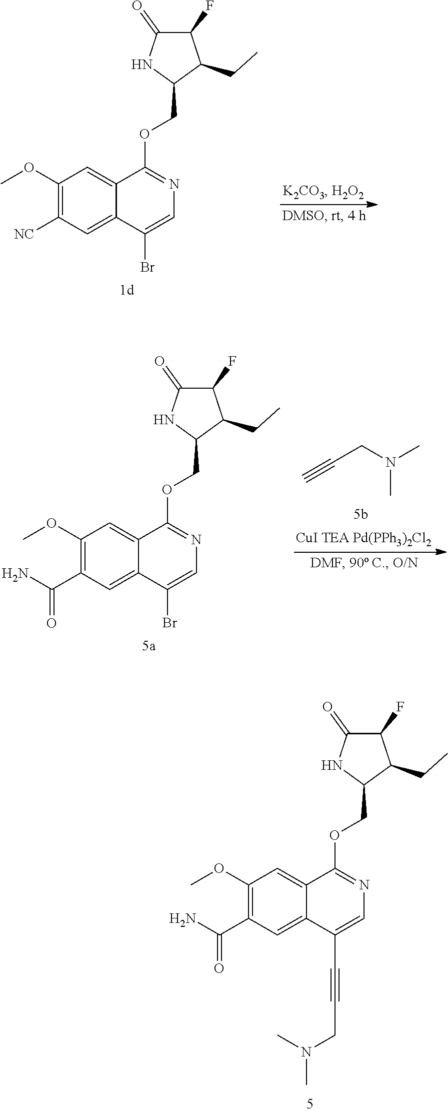

### Step 1. 4-bromo-1-(((2S,3S,4S)-3-ethyl-4-fluoro-5-oxopyrrolidin-2-yl)methoxy)-7-methoxyisoquinoline-6-carboxamide 5a

A mixture of 1d (97 mg, 0.23 mmol) and K₂CO₃ (127 mg, 0.92 mmol, Aldrich) in DMSO (3 mL, Aldrich) was treated with H₂O₂ (30%, 235 uL, 2.3 mmol, Aldrich) added dropwise at rt. The mixture was stirred at rt for 4 h. After completion of the reaction, the mixture was poured into water and extracted with DCM. The DCM was separated, washed with water, brine, dried over Na₂SO₄, filtered, and concentrated. The residue was purified by chromatography to afford 70 mg (Yield: 70%) of the title compound 5a.

### Step 2. 4-(3-(dimethylamino)prop-1-yn-1-yl)-1-(((2S,3S,4S)-3-ethyl-4-fluoro-5-oxopyrrolidin-2-yl)methoxy)-7-methoxyisoquinoline-6-carboxamide 5

A mixture of 5a (37 mg, 0.084 mmol), N,N-dimethylprop-2-yn-1-amine 5b (1 ml, Aldrich), Pd(PPh₃)₂Cl₂ (6 mg, 0.0084 mmol, Aldrich), CuI (3 mg, 0.0168 mmol, Aldrich), TEA (1 mL, Aldrich) and DMF (3 mL, Aldrich) was flushed with N2 for 3 mins and then the mixture was stirred at 90° C. overnight. After completion of the reaction, the mixture was poured into water and extracted with DCM. The DCM was separated, washed with water, brine, dried over Na₂SO₄, filtered, and concentrated. The residue was purified by chromatography to afford 18 mg (Yield: 50%) of title compound 5. ¹H NMR (400 MHz, CDCl₃, ppm): δ 8.9 (s, 1H), 8.1 (s, 1H), 7.8 (brs, 1H), 7.6 (s, 1H), 7.1 (s, 1H), 6.1 (s, 1H), 5.0-4.9 (m, 1H), 4.9-4.7 (m, 1H), 4.5-4.3 (m, 1H), 4.2-4.1 (m, 1H), 4.0 (s, 3H), 3.6 (s, 2H), 2.8-2.5 (m, 1H), 2.4 (s, 6H), 1.9-1.5 (m, 2H), 1.1-0.9 (m, 3H). MS m/z (ESI): 443 [M+1].

### Example 6: 1-(((2S,3S,4S)-3-ethyl-4-fluoro-5-oxopyrrolidin-2-yl)methoxy)-7-methoxy-4-(3-methoxyprop-1-yn-1-yl)isoquinoline-6-carboxamide 6

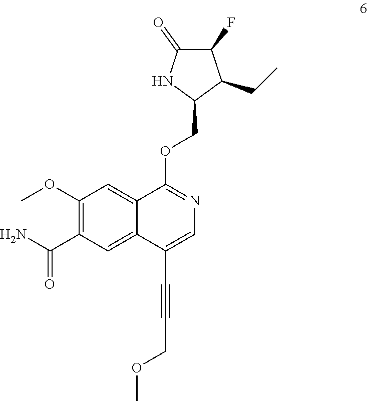 [SMILES: CC[C@@H]1[C@H](F)C(=O)N[C@@H]1COc1ncc(C#CCOC)c2cc(C(N)=O)c(OC)cc12]

This compound was prepared by the same method as of example 3 to afford the product. ¹H NMR (400 MHz, CDCl₃, ppm): δ 8.7 (s, 1H), 8.1 (s, 1H), 7.8 (brs, 1H), 7.7 (brs, 1H), 7.6 (s, 1H), 6.4 (brs, 1H), 5.0-4.7 (m, 2H), 4.6-4.4 (m, 3H), 4.3-4.1 (m, 1H), 4.0 (s, 3H), 3.6 (s, 3H), 2.7-2.5 (m, 1H), 2.0-1.6 (m, 2H), 1.2-1.0 (m, 3H). MS m/z (ESI): 430 [M+1].

### Example 7: (S)-7-methoxy-1-((5-oxopyrrolidin-2-yl)methoxy)-4-(p-tolylethynyl)isoquinoline-6-carboxamide 7

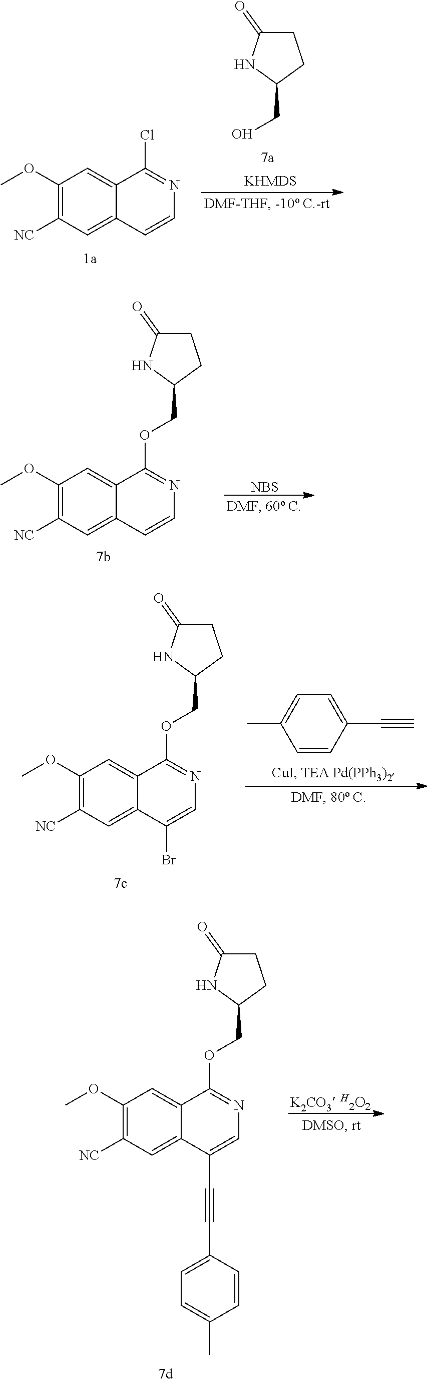

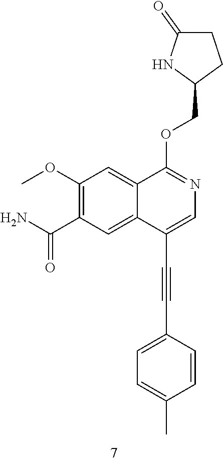 [SMILES: COc1cc2c(OC[C@@H]3CCC(=O)N3)ncc(C#Cc3ccc(C)cc3)c2cc1C(N)=O]

### Step 1. Synthesis of (S)-7-methoxy-1-((5-oxopyrrolidin-2-yl)methoxy)isoquinoline-6-carbonitrile 7b

A mixture of 1a (218 mg, 1.0 mmol, Pharmablock) and (S)-5-(hydroxymethyl)pyrrolidin-2-one 7a (127 mg, 1.1 mmol, Pharmablock) in DMF (5 mL, Aldrich) was treated with KHMDS (1 M in THF, 4.4 mL, Aldrich) at −10° C. Upon completion of the KHMDS addition, the cooling bath was removed, and the mixture was stirred at rt for 3 h. The reaction mixture was then poured into a mixture of 10% (w/v) NaH₂PO₄ and DCM with vigorous stirring. The DCM was separated, washed with water, brine, dried over Na₂SO₄, filtered, and concentrated. The residue was purified by chromatography to afford 116 mg (Yield: 40%) of the title compound 7b.

### Step 2. Synthesis of (S)-4-bromo-7-methoxy-1-((5-oxopyrrolidin-2-yl)methoxy)isoquinoline-6-carbonitrile 7c

To a stirred solution of 7b (116 mg, 0.39 mmol) in DMF (5 mL, Aldrich) at rt was added NBS (84 mg, 0.47 mmol, Aldrich) and the resulting mixture was stirred at 60° C. overnight. After completion of the reaction, the mixture was poured into water and extracted with DCM. The DCM was separated, washed with water, brine, dried over Na₂SO₄, filtered, and concentrated. The residue was purified by chromatography to afford 101 mg (Yield: 70%) of the title compound 7c.

### Step 3. (S)-7-methoxy-1-((5-oxopyrrolidin-2-yl)methoxy)-4-(p-tolylethynyl)isoquinoline-6-carbonitrile 7d

A mixture of 7c (60 mg, 0.16 mmol), 1-ethynyl-4-methylbenzene (1 mL, Aldrich), Pd(PPh₃)₂Cl₂ (11 mg, 0.016 mmol, Aldrich), CuI (6 mg, 0.032 mmol, Aldrich), TEA (1 mL, Aldrich) and DMF (3 mL, Aldrich) was flushed with N2 for 3 mins and then the mixture was stirred at 90° C. overnight. After completion of the reaction, the mixture was poured into water and extracted with DCM. The DCM was separated, washed with water, brine, dried over Na₂SO₄, filtered, and concentrated. The residue was purified by chromatography to afford 50 mg (Yield: 76%) of the title compound 7d.

### Step 4. (S)-7-methoxy-1-((5-oxopyrrolidin-2-yl)methoxy)-4-(p-tolylethynyl)isoquinoline-6-carboxamide 7

A mixture of 7d (50 mg, 0.12 mmol) and K₂CO₃ (67 mg, 0.48 mmol, Aldrich) in DMSO (3 mL, Aldrich) was treated with H₂O₂ (30%, 124 uL, 1.2 mmol, Aldrich) added dropwise at rt. The mixture was stirred at rt for 4 h. After completion of the reaction, the mixture was poured into water and extracted with DCM. The DCM was separated, washed with water, brine, dried over Na₂SO₄, filtered, and concentrated. The residue was purified by chromatography to afford 45 mg (Yield: 86%) of the title compound 7. ¹H NMR (400 MHz, CDCl₃, ppm): δ 9.1 (s, 1H), 8.1 (s, 1H), 7.7 (brs, 1H), 7.6-7.4 (m, 3H), 7.2-7.1 (m, 2H), 5.9-5.7 (m, 2H), 4.7-4.6 (m, 1H), 4.5-4.3 (m, 1H), 4.2-4.1 (m, 1H), 4.1 (s, 3H), 2.5-2.3 (m, 5H), 2.1-1.9 (m, 1H). MS m/z (ESI): 430 [M+1].

### Example 8: (S)-4-(3-(dimethylamino)prop-1-yn-1-yl)-7-methoxy-1-((5-oxopyrrolidin-2-yl)methoxy)isoquinoline-6-carboxamide 8

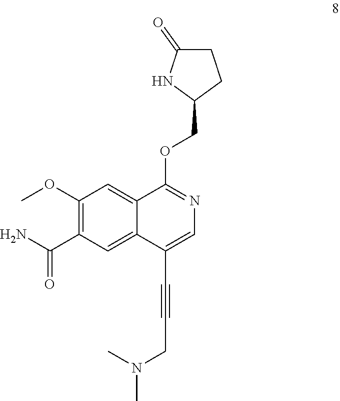 [SMILES: COc1cc2c(OC[C@@H]3CCC(=O)N3)ncc(C#CCN(C)C)c2cc1C(N)=O]

This compound was prepared by the same method as of example 7 to afford the product. ¹H NMR (400 MHz, CDCl₃, ppm): δ 8.7 (s, 1H), 8.1 (s, 1H), 7.7 (s, 1H), 4.6-4.3 (m, 3H), 4.2 (s, 3H), 3.6 (s, 2H), 2.6-2.4 (m, 9H), 2.2-2.0 (m, 1H). MS m/z (ESI): 397 [M+1].

### Example 9: (S)-7-methoxy-4-(3-methoxyprop-1-yn-1-yl)-1-((5-oxopyrrolidin-2-yl)methoxy)isoquinoline-6-carboxamide 9

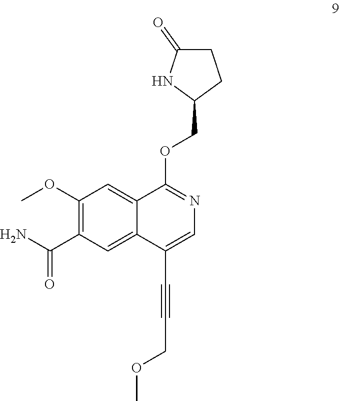 [SMILES: COCC#Cc1cnc(OC[C@@H]2CCC(=O)N2)c2cc(OC)c(C(N)=O)cc12]

This compound was prepared by the same method as of example 7 to afford the product. ¹H NMR (400 MHz, CDCl₃, ppm): δ 8.5 (s, 1H), 8.0 (s, 1H), 7.6 (s, 1H), 4.6-4.3 (m, 4H), 4.2-4.1 (m, 1H), 4.0 (s, 3H), 3.4 (s, 3H), 2.5-2.3 (m, 3H), 2.1-1.9 (m, 1H). MS m/z (ESI): 384 [M+1].

### Example 10: 4-((2H-tetrazol-5-yl)ethynyl)-1-(((2S,3S,4S)-3-ethyl-4-fluoro-5-oxopyrrolidin-2-yl)methoxy)-7-methoxyisoquinoline-6-carboxamide

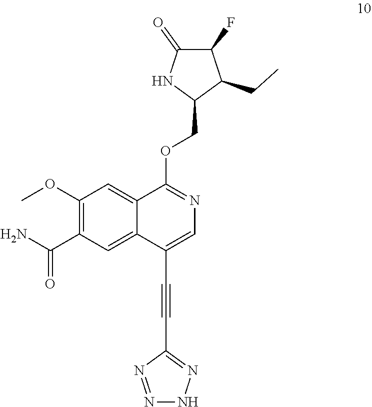 [SMILES: CC[C@@H]1[C@H](F)C(=O)N[C@@H]1COc1ncc(C#Cc2nn[nH]n2)c2cc(C(N)=O)c(OC)cc12]

This compound was prepared by the same method as of example 3 to afford the product. ¹H NMR (400 MHz, CDCl₃, ppm): δ 8.5 (s, 1H), 8.2 (s, 1H), 7.8 (s, 1H), 7.5 (brs, 1H), 7.4 (brs, 1H), 7.2 (brs, 1H), 5.0 (m, 1H), 4.7 (m, 1H), 4.4 (m, 1H), 4.2 (m, 1H), 4.0 (s, 3H), 2.7-2.5 (m, 1H), 1.8-1.6 (m, 2H), 1.2-1.1 (m, 3H). MS m/z (ESI): 454 [M+1].

### Example 11: 4-(3-(dimethylamino)-3-oxoprop-1-yn-1-yl)-1-(((2S,3S,4S)-3-ethyl-4-fluoro-5-oxopyrrolidin-2-yl)methoxy)-7-methoxyisoquinoline-6-carboxamide 11

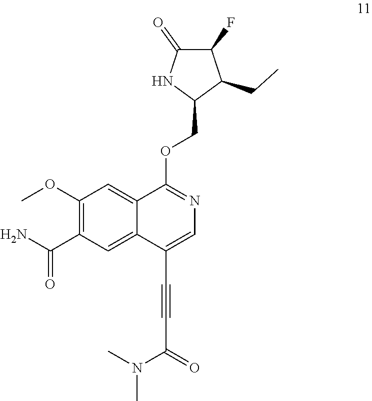 [SMILES: CC[C@@H]1[C@H](F)C(=O)N[C@@H]1COc1ncc(C#CC(=O)N(C)C)c2cc(C(N)=O)c(OC)cc12]

This compound was prepared by the same method as of example 1 to afford the product. ¹H NMR (400 MHz, CDCl₃, ppm): δ 8.6 (s, 1H), 8.1 (s, 1H), 7.9 (brs, 1H), 7.7 (brs, 1H), 7.6 (s, 1H), 6.3 (brs, 1H), 5.0-4.8 (m, 1H), 4.8-4.7 (m, 1H), 4.5-4.4 (m, 1H), 4.3-4.2 (m, 1H), 4.0 (s, 3H), 3.4 (s, 3H), 3.1 (s, 3H), 2.7-2.5 (m, 1H), 1.8-1.6 (m, 2H), 1.2-1.1 (m, 3H). MS m/z (ESI): 457 [M+1].

### Example 12: 1-(((2S,3S,4S)-3-ethyl-4-fluoro-5-oxopyrrolidin-2-yl)methoxy)-7-methoxy-4-(pyrimidin-5-ylethynyl)isoquinoline-6-carboxamide 12

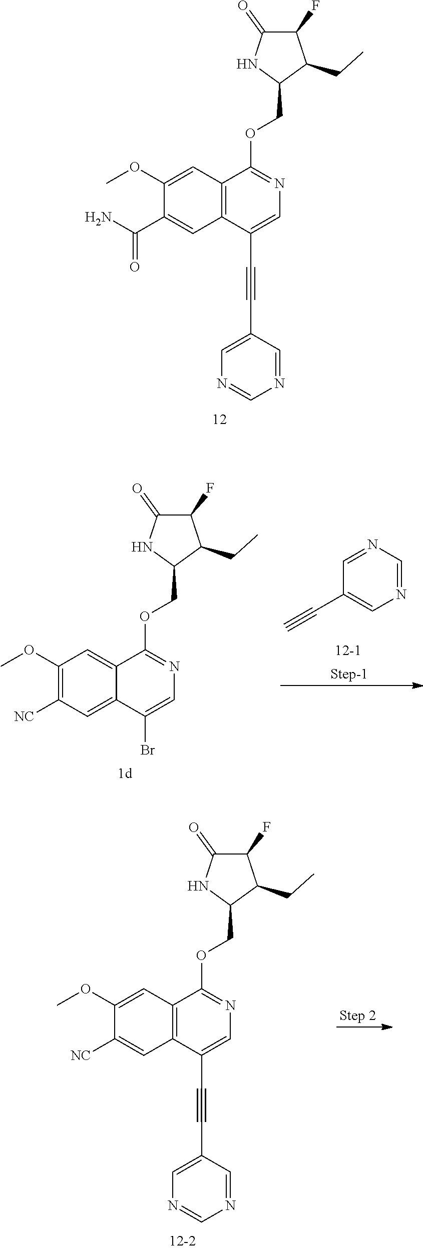

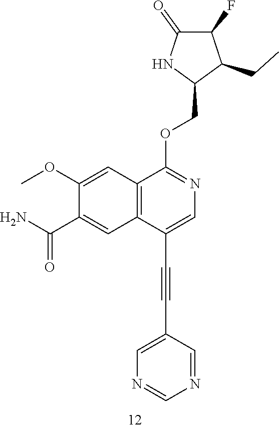 [SMILES: CC[C@@H]1[C@H](F)C(=O)N[C@@H]1COc1ncc(C#Cc2cncnc2)c2cc(C(N)=O)c(OC)cc12]

### Step 1. Synthesis of 1-(((2S,3S,4S)-3-ethyl-4-fluoro-5-oxopyrrolidin-2-yl)methoxy)-7-methoxy-4-(pyrimidin-5-ylethynyl)isoquinoline-6-carbonitrile 12-2

The mixture of 1d (60 mg, 142.10 umol), 5-ethynylpyrimidine 12-1 (73.97 mg, 710.48 umol, Bidepharm), Pd(PPh₃)₂Cl₂ (6 mg, 14.21 umol), CuI (2.71 mg, 14.21 umol) and TEA (43.14 mg, 426.29 umol) in DMF (2 mL) was stirred at 90° C. for 15 hours under N₂. The mixture was diluted with water and then extracted with EA (100 mL). The organic solution was dried and concentrated. The residue was purified by SGC (DCM:MeOH=50:1) to give the title compound 12-2 (50 mg, 112.25 umol, 78.99% yield). MS m/z (ESI): 446.1 [M+H]⁺.

### Step 2. Synthesis of 1-(((2S,3S,4S)-3-ethyl-4-fluoro-5-oxopyrrolidin-2-yl)methoxy)-7-methoxy-4-(pyrimidin-5-ylethynyl)isoquinoline-6-carboxamide 12

The mixture of 12-2 (50 mg, 112.25 umol) and K₂CO₃ (100 mg, 336.74 umol) in H₂O₂ (30% in water, 0.2 mL) and DMSO (2 mL) was stirred at rt for 3 hours. The mixture was diluted with water and then extracted with EA (100 mL). The organic solution was dried and concentrated. The crude was triturated with MeOH (3 mL) and filtered, and the filter cake was dried in vacuo to give the title compound 12 (20 mg, 43.15 umol, 38.45% yield).

¹H NMR (400 MHz, DMSO-d₆, ppm) δ 9.24 (s, 1H), 9.12 (s, 2H), 8.90 (brs, 1H), 8.37 (s, 1H), 8.32 (s, 1H), 7.93 (brs, 1H), 7.82 (s, 1H), 7.79 (brs, 1H), 4.93 (dd, 1H), 4.60 (dd, 1H), 4.33 (dd, 1H), 4.12 (br, 1H), 4.00 (s, 3H), 2.65-2.53 (m, 1H), 1.64-1.58 (m, 2H), 1.03 (t, 3H). MS m/z (ESI): 464.1 [M+H]⁺.

### Example 13: 1-(((2S,3S,4S)-3-ethyl-4-fluoro-5-oxopyrrolidin-2-yl)methoxy)-7-methoxy-4-(pyrazin-2-ylethynyl)isoquinoline-6-carboxamide 13

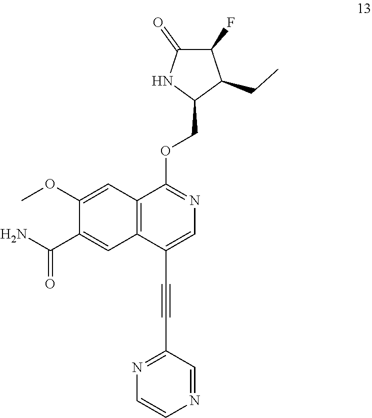 [SMILES: CC[C@@H]1[C@H](F)C(=O)N[C@@H]1COc1ncc(C#Cc2cnccn2)c2cc(C(N)=O)c(OC)cc12]

This compound was prepared by essential the same method as of example 12 to afford the title compound 13 (30 mg, 64.73 umol, 48.06% yield).

¹H NMR (400 MHz, DMSO-d6, ppm) δ 8.98 (d, 1H), 8.91 (s, 1H), 8.75 (dd, 1H), 8.69 (d, 1H), 8.38 (s, 1H), 8.37 (s, 1H), 7.96 (s, 1H), 7.83 (brs, 2H), 4.91 (dd, 6.0 Hz, 1H), 4.62 (dd, 1H), 4.33 (dd, 1H), 4.13 (br, 1H), 4.00 (s, 3H), 2.69-2.57 (m, 1H), 1.64-1.57 (m, 2H), 1.03 (t, 3H). MS m/z (ESI): 464.2 [M+H]⁺.

### Example 14: 1-(((2S,3S,4S)-3-ethyl-4-fluoro-5-oxopyrrolidin-2-yl)methoxy)-7-methoxy-4-(pyridin-3-ylethynyl)isoquinoline-6-carboxamide 14

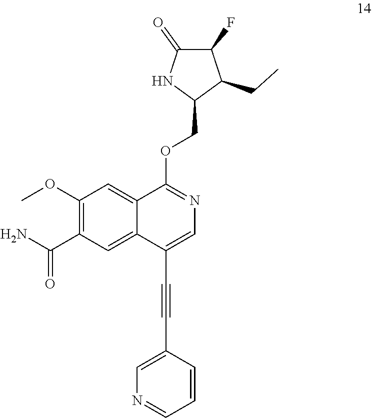 [SMILES: CC[C@@H]1[C@H](F)C(=O)N[C@@H]1COc1ncc(C#Cc2cccnc2)c2cc(C(N)=O)c(OC)cc12]

This compound was prepared by essential the same method as of example 12 to afford the title compound 14 (40 mg, 86.49 umol, 72.53% yield).

¹H NMR (400 MHz, DMSO-d₆, ppm) δ 8.91 (s, 1H), 8.86 (s, 1H), 8.63 (s, 1H), 8.39 (s, 1H), 8.29 (s, 1H), 8.08 (d, 1H), 7.94 (s, 1H), 7.82 (s, 1H), 7.81 (d, 1H), 7.54-7.50 (m, 1H), 4.91 (dd, 1H), 4.60 (dd, 1H), 4.32 (dd, 1H), 4.12 (brs, 1H), 4.00 (s, 3H), 2.70-2.57 (m, 1H), 1.61 (br, 2H), 1.03 (t, 3H). LCMS: MS m/z (ESI): 463.1 [M+H]⁺.

### Example 15: 8-(3-(dimethylamino)prop-1-yn-1-yl)-5-(((2S,3S,4S)-3-ethyl-4-fluoro-5-oxopyrrolidin-2-yl)methoxy)-3-methoxy-2-naphthamide 15

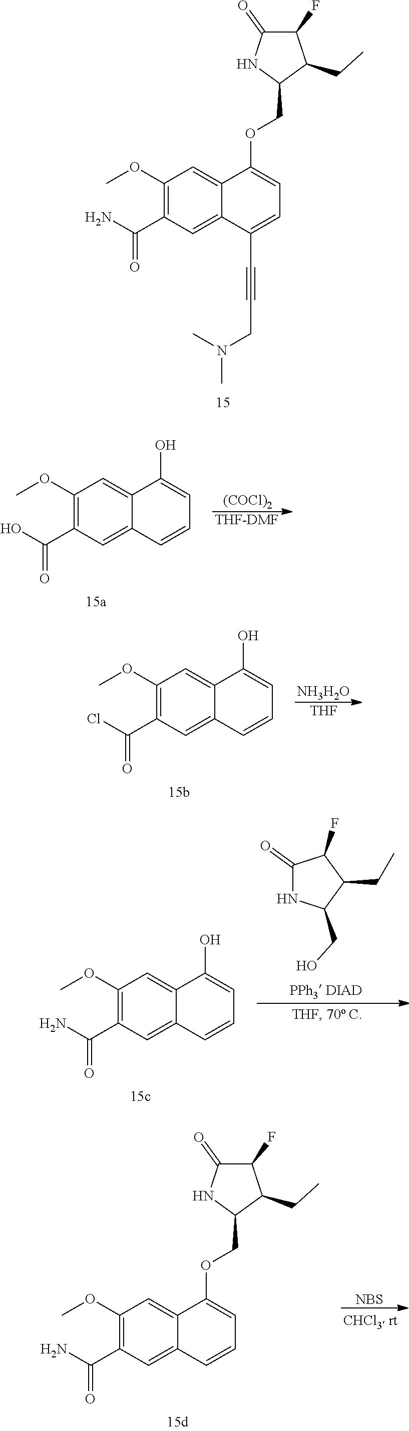

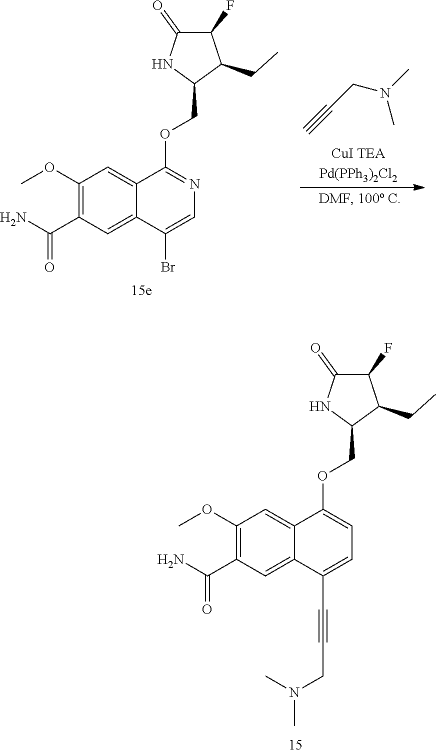

### Step 1 and 2. 5-hydroxyl-3-methoxy-2-naphthamide 15c

A suspension of 5-hydroxyl-3-methoxy-2-naphthoic acid 15a (500 mg, 2.3 mmol, Abovchem) in a mixture of DMF (0.094 mL, Aldrich) and THF (12.5 mL, Aldrich) was treated with oxalyl chloride (0.4 mL, Aldrich) at rt. The resulting suspension was stirred for 1 h. Then it was concentrated to dryness and the residue was suspended in THE and cooled in ice. Concentrated ammonia (2 mL) was added over a period of about 5 min. The mixture was then stirred at rt for 1 h. The solvent was removed, and water was added, and the precipitate was filtered, washed with water and dried under vacuum. The resulting residue was suspended in ethyl acetate and heated under reflux for 2 h. The residue was filtered, washed with ethyl acetate and dried to give the title compound 15c (450 mg, Yield: 90%).

### Step 3. 5-(((2S,3S,4S)-3-ethyl-4-fluoro-5-oxopyrrolidin-2-yl)methoxy)-3-methoxy-2-naphthamide 15d

A mixture of 15c (217 mg, 1 mmol), 1b (161 mg, 1 mmol, Pharmablock) and PPh₃ (707 mg, 2.7 mmol, Aldrich) was stirred at rt, DIAD (384 mg, 1.9 mmol, Alfa Aesar) was then added. The resulting reaction was heated at 70° C. for 20 h. After completion of reaction, the mixture was concentrated to dryness and purified by chromatography to afford 180 mg (Yield: 50%) of the title compound 15d.

### Step 4. 8-bromo-5-(((2S,3S,4S)-3-ethyl-4-fluoro-5-oxopyrrolidin-2-yl)methoxy)-3-methoxy-2-naphthamide 15e

To a solution of 15d (72 mg, 0.2 mmol) in CHCl₃ (5 mL, Aldrich) was added NBS (39 mg, 0.22 mmol, Aldrich) at rt. The mixture was stirred at rt for 6 h. After completion of reaction, the mixture was concentrated to dryness and purified by chromatography to afford 74 mg (Yield: 84%) of the title compound 15e.

### Step 5. 8-(3-(dimethylamino)prop-1-yn-1-yl)-5-(((2S,3S,4S)-3-ethyl-4-fluoro-5-oxopyrrolidin-2-yl)methoxy)-3-methoxy-2-naphthamide 15

A mixture of 15e (37 mg, 0.084 mmol), N,N-dimethylprop-2-yn-1-amine (1 mL, Aldrich), Pd(PPh₃)₂Cl₂ (6 mg, 0.0084 mmol, Aldrich), CuI (3 mg, 0.017 mmol, Aldrich), TEA (1 mL, Aldrich) and DMF (3 mL, Aldrich) was flushed with N₂ for 3 mins and then the mixture was stirred at 90° C. overnight. After completion of the reaction, the mixture was poured into water and extracted with DCM. The DCM was separated, washed with water, brine, dried over Na₂SO₄, filtered, and concentrated. The residue was purified by chromatography to afford 20 mg (Yield: 54%) of the title compound 15. ¹H NMR (400 MHz, CDCl₃, ppm): δ 9.0 (s, 1H), 7.8 (brs, 1H), 7.7-7.4 (m, 3H), 6.8-6.6 (m, 1H), 6.1 (brs, 1H), 5.0-4.8 (m, 1H), 4.3-4.1 (m, 3H), 4.0 (s, 3H), 3.6 (s, 2H), 2.8-2.5 (m, 1H), 2.5 (s, 6H), 1.8-1.6 (m, 2H), 1.2-1.0 (m, 3H). MS m/z (ESI): 442 [M+1].

### Example 16: 1-(((2S,3S,4S)-3-ethyl-4-fluoro-5-oxopyrrolidin-2-yl)methoxy)-7-methoxy-4-(pyrimidin-2-ylethynyl)isoquinoline-6-carboxamide 16

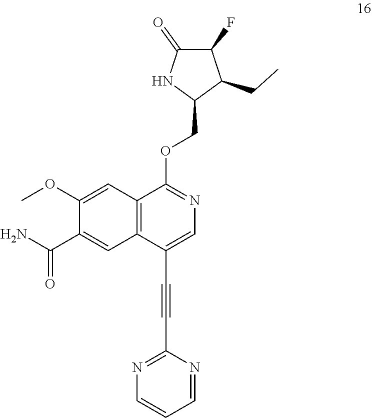 [SMILES: CC[C@@H]1[C@H](F)C(=O)N[C@@H]1COc1ncc(C#Cc2ncccn2)c2cc(C(N)=O)c(OC)cc12]

This compound was prepared by essential the same method as of example 12 to afford the title compound 16 (10 mg, 21.58 umol, 32.04% yield).

¹HNMR (400 MHz, DMSO-d₆, ppm) δ 8.90 (d, 3H), 8.38 (d, 2H), 7.96 (brs, 1H), 7.83 (s, 2H), 7.56 (t, 1H), 4.91 (dd, 1H), 4.62 (dd, 1H), 4.35 (dd, 1H), 4.13 (brs, 1H), 4.01 (s, 3H), 2.71-2.51 (m, 1H), 1.66-1.58 (m, 2H), 1.03 (t, 3H). MS m/z (ESI): 464.5 [M+H]⁺.

### Example 17: 1-(((2S,3S,4S)-3-ethyl-4-fluoro-5-oxopyrrolidin-2-yl)methoxy)-7-methoxy-4-((1-methyl-1H-pyrazol-4-yl)ethynyl)isoquinoline-6-carboxamide 17

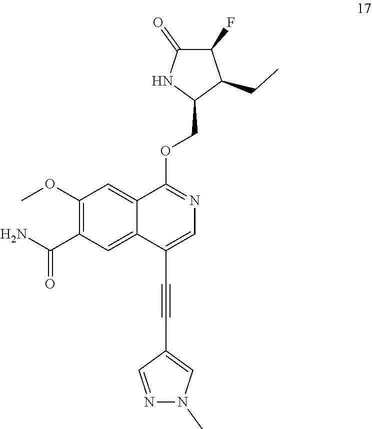 [SMILES: CC[C@@H]1[C@H](F)C(=O)N[C@@H]1COc1ncc(C#Cc2cnn(C)c2)c2cc(C(N)=O)c(OC)cc12]

This compound was prepared by essential the same method as of example 12 to afford the title compound 17 (20 mg, yield: 32%).

¹H NMR (400 MHz, DMSO-d₆, ppm) δ 8.90 (s, 1H), 8.31 (s, 1H), 8.16 (d, 2H), 7.92 (s, 1H), 7.79 (s, 1H), 7.76 (s, 1H), 4.91 (dd, 1H), 4.62 (dd, Hz, 1H), 4.33 (dd, 1H), 4.11 (br, 1H), 3.99 (s, 3H), 3.88 (s, 3H), 2.70-2.56 (m, 1H), 1.63-1.55 (m, 2H), 1.02 (t, 3H). MS m/z (ESI): 466.1 [M+H]⁺.

### Example 18: 1-(((2S,3S,4S)-3-ethyl-4-fluoro-5-oxopyrrolidin-2-yl)methoxy)-4-((S)-3-hydroxybut-1-yn-1-yl)-7-methoxyisoquinoline-6-carboxamide 18

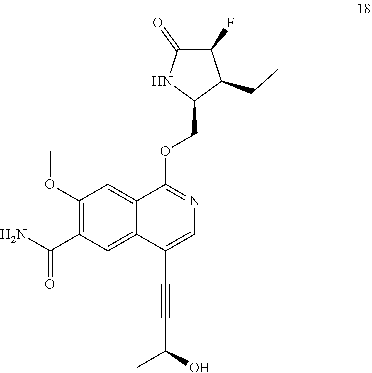 [SMILES: CC[C@@H]1[C@H](F)C(=O)N[C@@H]1COc1ncc(C#C[C@H](C)O)c2cc(C(N)=O)c(OC)cc12]

This compound was prepared by essential the same method as of example 12 to afford the title compound 18 (20 mg, 46.57 umol, 31.93% yield).

¹H NMR (400 MHz, DMSO-d₆, ppm) δ 8.87 (s, 1H), 8.28 (s, 1H), 8.07 (s, 1H), 7.90 (s, 1H), 7.77 (s, 1H), 7.76 (d, 1H), 5.62 (d, 1H), 4.91 (dd, 1H), 4.75-4.70 (m, 1H), 4.56 (dd, 1H), 4.27 (dd, 1H), 4.10 (br, 1H), 3.98 (s, 3H), 2.68-2.52 (m, 1H), 1.63-1.55 (m, 2H), 1.46 (d, 3H), 1.02 (t, 3H). MS m/z (ESI): 430.1 [M+H]⁺.

### Example 19: 1-(((2S,3S,4S)-3-ethyl-4-fluoro-5-oxopyrrolidin-2-yl)methoxy)-4-((R)-3-hydroxybut-1-yn-1-yl)-7-methoxyisoquinoline-6-carboxamide 19

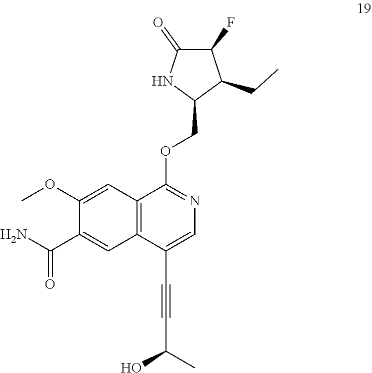 [SMILES: CC[C@@H]1[C@H](F)C(=O)N[C@@H]1COc1ncc(C#C[C@@H](C)O)c2cc(C(N)=O)c(OC)cc12]

This compound was prepared by essential the same method as of example 12 to afford the title compound 19 (30 mg, 69.86 umol, 47.90% yield).

¹H NMR (400 MHz, DMSO-d₆, ppm) δ 8.88 (s, 1H), 8.29 (s, 1H), 8.07 (s, 1H), 7.90 (s, 1H), 7.77 (brs, 2H), 5.61 (d, 1H), 4.91 (dd, 1H), 4.75-4.69 (m, 1H), 4.55 (dd, 1H), 4.28 (dd, 1H), 4.10 (br, 1H), 3.98 (s, 3H), 2.70-2.56 (m, 1H), 1.63-1.55 (m, 2H), 1.46 (d, 3H), 1.02 (t, 3H). MS m/z (ESI): 430.1 [M+H]⁺.

### Example 20: 4-((1H-pyrazol-4-yl)ethynyl)-1-(((2S,3S,4S)-3-ethyl-4-fluoro-5-oxopyrrolidin-2-yl)methoxy)-7-methoxyisoquinoline-6-carboxamide 20

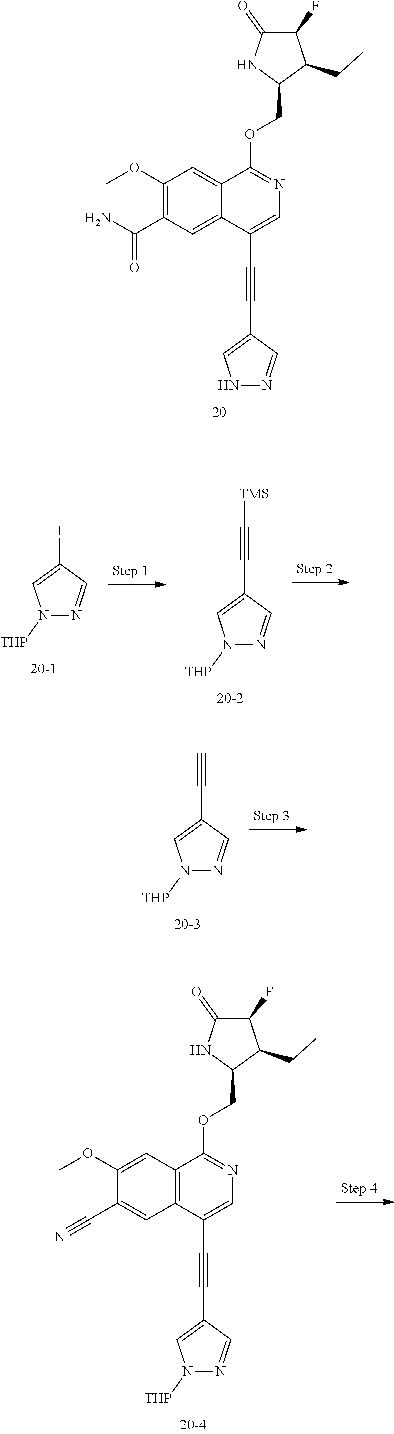

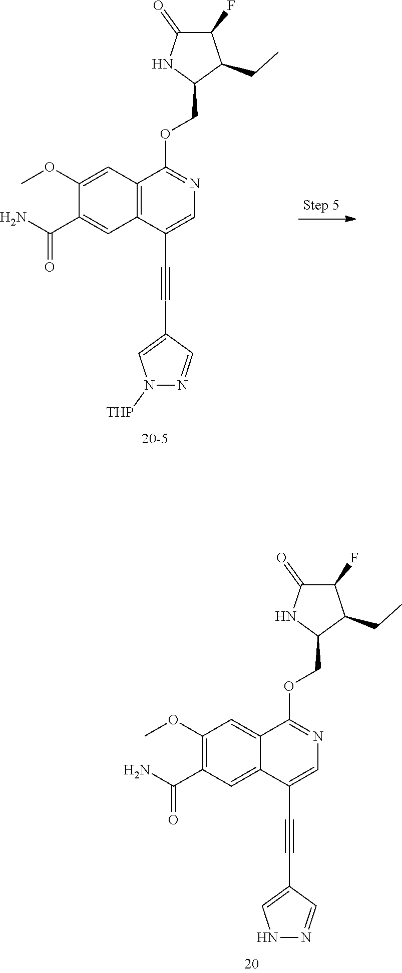

### Step 1. Synthesis of 1-(tetrahydro-2H-pyran-2-yl)-4-((trimethylsilyl)ethynyl)-1H-pyrazole 20-2

To a stirred mixture of 4-iodo-1-(tetrahydro-2H-pyran-2-yl)-1H-pyrazole 20-1 (6 g, 21.58 mmol, Bidepharm), CuI (205.46 mg, 1.08 mmol), dichloropalladium; triphenylphosphane (757.20 mg, 1.08 mmol) and triethylamine (6.55 g, 64.73 mmol) in DMF (60 mL) at rt under N₂ was added ethynyl(trimethyl)silane (6.36 g, 64.73 mmol). The mixture was stirred at this temperature overnight. To the mixture was added EtOAc (200 mL), the organic layer was washed with H₂O (200 mL×3). The organic layer was dried, concentrated, the residue was purified by silica gel chromatography (PE/EA=50/1) to give the title compound 20-2 (3.5 g, 14.09 mmol, 65.31% yield). ¹H NMR (400 MHz, CDCl₃, ppm) δ 7.75 (s, 1H), 7.62 (s, 1H), 5.36-5.30 (m, 1H), 4.03-4.01 (m, 1H), 3.71-3.66 (m, 1H), 2.07-1.99 (m, 3H), 1.69-1.58 (m, 3H), 0.22 (s, 9H).

### Step 2. Synthesis of 4-ethynyl-1-(tetrahydro-2H-pyran-2-yl)-1H-pyrazole 20-3

To a stirred solution of 20-2 (3.3 g, 13.29 mmol) in DCM (35 mL) at 0° C. was added tetrabutylammonium; fluoride (17.37 g, 66.43 mmol). The mixture was stirred at rt for 1 h. To the mixture was added H₂O (60 mL), the aqueous layer was extracted with DCM (20 mL×4). The combined organic layer was dried, concentrated, and the residue was purified by silica gel chromatography (PE/EA=10/1) to give the title compound 20-3 (2.0 g, 11.35 mmol, 85.43% yield). MS m/z (ESI): 177.2 [M+H]+

### Step 3. Synthesis of 1-(((2S,3S,4S)-3-ethyl-4-fluoro-5-oxopyrrolidin-2-yl)methoxy)-7-methoxy-4-((1-(tetrahydro-2H-pyran-2-yl)-1H-pyrazol-4-yl)ethynyl)isoquinoline-6-carbonitrile 20-4

The mixture of 20-3 (187.80 mg, 1.07 mmol), 4-bromo-1-(((2S,3S,4S)-3-ethyl-4-fluoro-5-oxopyrrolidin-2-yl)methoxy)-7-methoxyisoquinoline-6-carbonitrile (1d, 150 mg, 355.24 umol), CuI (6.77 mg, 35.52 umol), dichloropalladium; triphenylphosphane (24.93 mg, 35.52 umol) and triethylamine (179.73 mg, 1.78 mmol) in DMF (5 mL) was heated to 90° C. overnight under N₂. The mixture was concentrated in high vacuo and the residue was purified by silica gel chromatography (EtOAc) to give the title compound 20-4 (180 mg, 347.79 umol, 97.90% yield). MS m/z (ESI): 518.2 [M+H]⁺

### Step 4. Synthesis of 1-(((2S,3S,4S)-3-ethyl-4-fluoro-5-oxopyrrolidin-2-yl)methoxy)-7-methoxy-4-((1-(tetrahydro-2H-pyran-2-yl)-1H-pyrazol-4-yl)ethynyl)isoquinoline-6-carboxamide 20-5

To a stirred mixture of 20-4 (180 mg, 347.79 umol) and potassium carbonate (96.13 mg, 695.58 umol) in DMSO (2 mL) was added hydrogen peroxide (59.15 mg, 1.74 mmol, 30% in water). The mixture was stirred at rt for 2 h. To the mixture was added H₂O (12 mL), the resulting solid was filtered, and the solid was dried to give the title compound 20-5 (160 mg, 298.75 umol, 85.90% yield). MS m/z (ESI): 452.2 [M−THP+H]⁺

### Step 5. Synthesis of 4-((1H-pyrazol-4-yl)ethynyl)-1-(((2S,3S,4S)-3-ethyl-4-fluoro-5-oxopyrrolidin-2-yl)methoxy)-7-methoxyisoquinoline-6-carboxamide 20

To a stirred mixture of 20-5 (140 mg, 270.51 umol) in DCM (5 mL) at 0° C. was added TFA (616.88 mg, 5.41 mmol, 1 mL). The mixture was stirred at rt for 2 h and quenched with satd. aq. NaHCO₃ (15 mL). The aqueous layer was extracted with DCM (5 mL×4). The combined organic layer was dried, concentrated. The residue was purified by prep-HPLC to give the title compound 20 (45 mg, 99.68 umol, 36.85% yield).

¹H NMR (400 MHz, DMSO-d₆, ppm) δ 13.26 (brs, 1H), 8.89 (s, 1H), 8.33 (s, 1H), 8.22 (brs, 1H), 8.16 (s, 1H), 7.92 (s, 1H), 7.79 (s, 2H), 4.91 (dd, 1H), 4.57 (dd, 1H), 4.29 (dd, 1H), 4.11 (brs, 1H), 3.99 (s, 3H), 2.72-2.51 (m, 1H), 1.65-1.56 (m, 2H), 1.02 (t, 3H). MS m/z (ESI): 452.2 [M+H]⁺.

### Example 21: 1-(((2S,3S,4S)-3-ethyl-4-fluoro-5-oxopyrrolidin-2-yl)methoxy)-7-methoxy-4-((2-methyl-2H-tetrazol-5-yl)ethynyl)isoquinoline-6-carboxamide 21

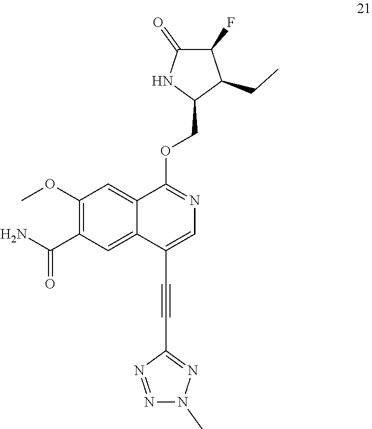 [SMILES: CC[C@@H]1[C@H](F)C(=O)N[C@@H]1COc1ncc(C#Cc2nnn(C)n2)c2cc(C(N)=O)c(OC)cc12]

This compound was prepared by the same method as of example 1 to afford the product. ¹H NMR (400 MHz, CDCl₃, ppm): δ 8.5 (s, 1H), 8.2 (s, 1H), 7.8 (s, 1H), 7.5 (brs, 1H), 7.4 (brs, 1H), 7.2 (brs, 1H), 5.0 (m, 1H), 4.7 (m, 1H), 4.4 (m, 1H), 4.2 (m, 1H), 4.0 (s, 3H), 3.6 (s, 3H), 2.7-2.5 (m, 1H), 1.8-1.6 (m, 2H), 1.2-1.1 (m, 3H). MS m/z (ESI): 468 [M+1]

### Example 22: 1-(((2S,3S,4S)-3-ethyl-4-fluoro-5-oxopyrrolidin-2-yl)methoxy)-7-methoxy-4-(pyridin-4-ylethynyl)isoquinoline-6-carboxamide 22

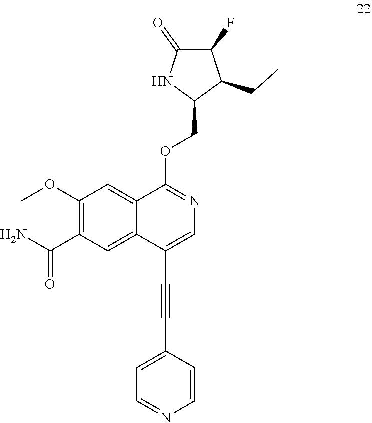 [SMILES: CC[C@@H]1[C@H](F)C(=O)N[C@@H]1COc1ncc(C#Cc2ccncc2)c2cc(C(N)=O)c(OC)cc12]

This compound was prepared by essential the same method as of example 12 to afford the title compound 22 (25 mg, 54.06 umol, 38.14% yield).

¹H NMR (400 MHz, DMSO-d₆, ppm) δ 8.90 (s, 1H), 8.68 (dd, 2H), 8.37 (s, 1H), 8.32 (s, 1H), 7.95 (s, 1H), 7.82 (s, 1H), 7.81 (d, 1H), 7.62 (dd, 2H), 4.91 (dd, 1H), 4.60 (dd, 1H), 4.32 (dd, 1H), 4.12 (brs, 1H), 4.00 (s, 3H), 2.71-2.57 (m, 1H), 1.65-1.56 (m, 2H), 1.03 (t, 3H). MS m/z (ESI): 463.1 [M+H]⁺.

### Example 23: 1-(((2S,3S,4S)-3-ethyl-4-fluoro-5-oxopyrrolidin-2-yl)methoxy)-7-methoxy-4-(pyridin-2-ylethynyl)isoquinoline-6-carboxamide 23

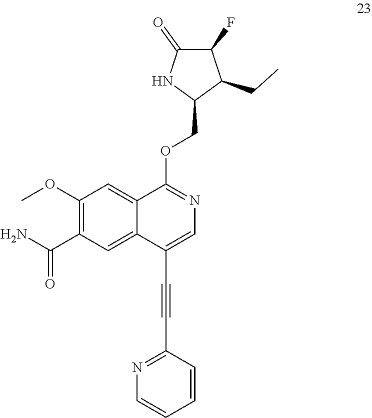 [SMILES: CC[C@@H]1[C@H](F)C(=O)N[C@@H]1COc1ncc(C#Cc2ccccn2)c2cc(C(N)=O)c(OC)cc12]

This compound was prepared by essential the same method as of example 12.

¹H NMR (400 MHz, DMSO-d₆, ppm) δ 8.92 (s, 1H), 8.67 (d, 1H), 8.38 (s, 1H), 8.32 (s, 1H), 7.96-7.94 (m, 1H), 7.92 (dd, 1H), 7.82 (s, 2H), 7.75 (d, 1H), 7.50-7.45 (m, 1H), 4.91 (dd, 1H), 4.61 (dd, 1H), 4.32 (dd, 1H), 4.12 (brs, 1H), 4.00 (s, 3H), 2.71-2.57 (m, 1H), 1.65-1.56 (m, 2H), 1.03 (t, 3H). MS m/z (ESI): 463.1 [M+H]⁺.

### Example 24: 4-((4-cyanophenyl)ethynyl)-1-(((2S,3S,4S)-3-ethyl-4-fluoro-5-oxopyrrolidin-2-yl)methoxy)-7-methoxyisoquinoline-6-carboxamide 24

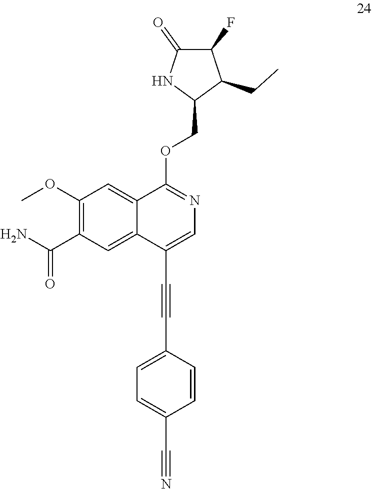 [SMILES: CC[C@@H]1[C@H](F)C(=O)N[C@@H]1COc1ncc(C#Cc2ccc(C#N)cc2)c2cc(C(N)=O)c(OC)cc12]

This compound was prepared by essential the same method as of example 12.

¹H NMR (400 MHz, DMSO-d₆, ppm) δ 8.90 (s, 1H), 8.37 (s, 1H), 8.31 (s, 1H), 7.96 (s, 1H), 7.95 (d, 2H), 7.85 (s, 1H), 7.84-7.81 (m, 2H), 7.79 (s, 1H), 4.91 (dd, 1H), 4.60 (dd, 1H), 4.32 (dd, 1H), 4.12 (brs, 1H), 4.00 (s, 3H), 2.68-2.57 (m, 1H), 1.63-1.56 (m, 2H), 1.02 (t, 3H). MS m/z (ESI): 487.1 [M+H]⁺.

### Example 25:1-(((2S,3S,4S)-3-ethyl-4-fluoro-5-oxopyrrolidin-2-yl)methoxy)-7-isopropoxy-4-(3-methoxyprop-1-yn-1-yl)isoquinoline-6-carboxamide 25

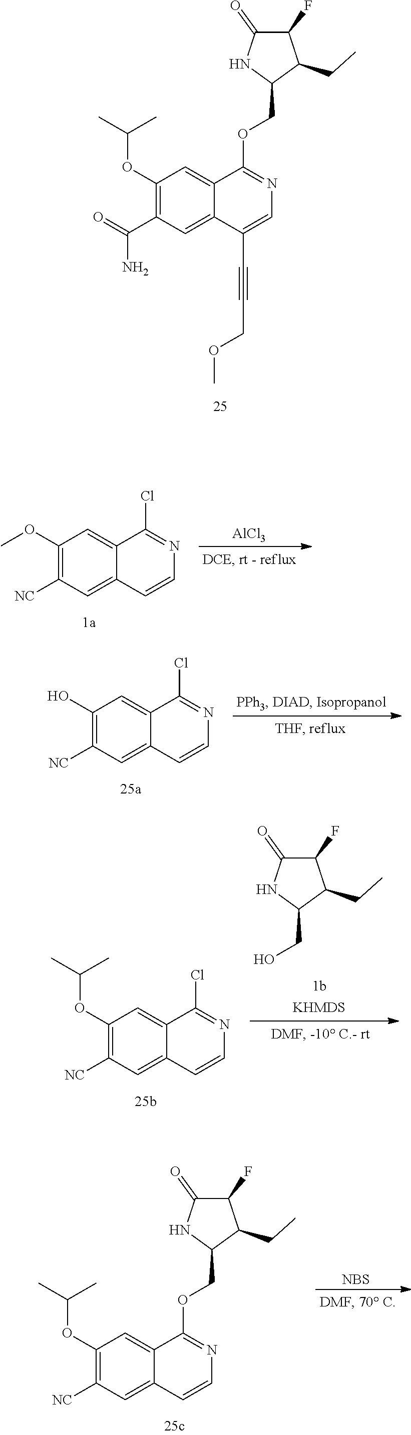

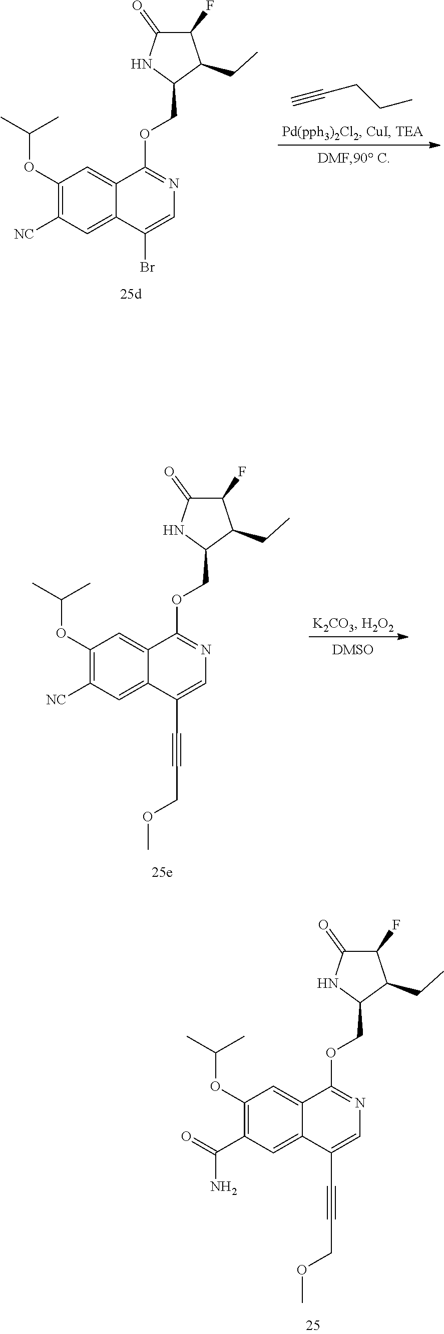

### Step 1. Synthesis of 1-chloro-7-hydroxyisoquinoline-6-carbonitrile 25a

To a solution of 1a (400 mg, 1.83 mmol, Pharmablock) in DCE (Aldrich) was added AlCl₃ (732 mg, 5.5 mmol, Aldrich) at rt. The mixture was refluxed for 6 h. After completion of the reaction, the mixture was concentrated to dryness and purified by chromatography to afford 304 mg (Yield: 82%) of the title compound 25a.

### Step 2. Synthesis of 1-chloro-7-isopropoxyisoquinoline-6-carbonitrile 25b

A solution of PPh₃ (584 mg, 2.23 mmol, Aldrich) in THF (5 mL, Aldrich) was treated with DIAD (450 mg, 2.23 mmol, Alfa Aesar). After 5 min, isopropanol (1 mL, Aldrich) was added followed by 25a (304 mg, 1.49 mmol). The mixture was refluxed for 6 h, then cooled and concentrated. The residue was purified by chromatography to afford the title compound 25b (256 mg, Yield: 73%).

### Step 3. Synthesis of 1-(((2S,3S,4S)-3-ethyl-4-fluoro-5-oxopyrrolidin-2-yl)methoxy)-7-isopropoxyisoquinoline-6-carbonitrile 25c

A mixture of 25b (256 mg, 1.04 mmol) and 1b (183 mg, 1.14 mmol, Pharmablock) in DMF (10 mL, Aldrich) was treated with KHMDS (1 M in THF, 2.3 mL, Aldrich) at −10° C. Upon completion of the KHMDS addition, the cooling bath was removed, and the mixture was stirred at rt for 3 h. The reaction mixture was then poured into a mixture of 10% (w/v) NaH₂PO₄ and DCM with vigorous stirring. The DCM was separated, washed with water, brine, dried over Na₂SO₄, filtered, and concentrated. The residue was purified by chromatography to afford 124 mg (Yield: 35%) of the title compound 25c.

### Step 4. Synthesis of 4-bromo-1-(((2S,3S,4S)-3-ethyl-4-fluoro-5-oxopyrrolidin-2-yl)methoxy)-7-isopropoxyisoquinoline-6-carbonitrile 25d

To a stirred solution of 25c (124 mg, 0.33 mmol) in DMF (3 mL, Aldrich) at rt was added NBS (89 mg, 0.50 mmol, Aldrich) and the resulting mixture was stirred at 60° C. overnight. After completion of the reaction, the mixture was poured into water and extracted with DCM. The DCM was separated, washed with water, brine, dried over Na₂SO₄, filtered, and concentrated. The residue was purified by chromatography to afford 127 mg (Yield: 85%) of the title compound 25d

### Step 5. Synthesis of 1-(((2S,3S,4S)-3-ethyl-4-fluoro-5-oxopyrrolidin-2-yl)methoxy)-7-isopropoxy-4-(3-methoxyprop-1-yn-1-yl)isoquinoline-6-carbonitrile 25e

A mixture of 25d (80 mg, 0.177 mmol), 3-methoxyprop-1-yne (1 mL, Aldrich), Pd(PPh₃)₂Cl₂ (12 mg, 0.0177 mmol, Aldrich), CuI (7 mg, 0.0355 mmol, Aldrich), TEA (1 mL, Aldrich) and DMF (3 mL, Aldrich) was flushed with N₂ for 3 mins and then the mixture was stirred at 90° C. overnight. After completion of the reaction, the mixture was poured into water and extracted with DCM. The DCM was separated, washed with water, brine, dried over Na₂SO₄, filtered, and concentrated. The residue was purified by chromatography to afford 67 mg (Yield: 84%) of the title compound 25e.

### Step 6. Synthesis of 1-(((2S,3S,4S)-3-ethyl-4-fluoro-5-oxopyrrolidin-2-yl)methoxy)-7-isopropoxy-4-(3-methoxyprop-1-yn-1-yl)isoquinoline-6-carboxamide 25

A mixture of 25e (87 mg, 0.19 mmol) and K₂CO₃ (109 mg, 0.79 mmol, Aldrich) in DMSO (3 mL, Aldrich) was treated with H₂O₂ (30%, 202 uL, 1.98 mmol, Aldrich) added dropwise at rt. The mixture was stirred at rt for 4 h. After completion of the reaction, the mixture was poured into water and extracted with DCM. The DCM was separated, washed with water, brine, dried over Na₂SO₄, filtered, and concentrated. The residue was purified by chromatography to afford 58 mg (Yield: 65%) of the title compound 25. ¹H NMR (400 MHz, CDCl₃, ppm): δ 8.8 (s, 1H), 8.1 (s, 1H), 8.0 (brs, 1H), 7.7 (s, 1H), 7.5 (s, 1H), 6.4 (brs, 1H), 5.0-4.7 (m, 3H), 4.5 (s, 2H), 4.5-4.4 (m, 1H), 4.3-4.1 (m, 1H), 3.6 (s, 3H), 2.7-2.5 (m, 1H), 2.0-1.6 (m, 2H), 1.6-1.4 (m, 6H), 1.2-1.1 (m, 3H). MS m/z (ESI): 458 [M+1]

### Example 26: 1-(((2S,3S,4S)-3-ethyl-4-fluoro-5-oxopyrrolidin-2-yl)methoxy)-7-methoxy-4-((3-methoxyphenyl)ethynyl)isoquinoline-6-carboxamide 26

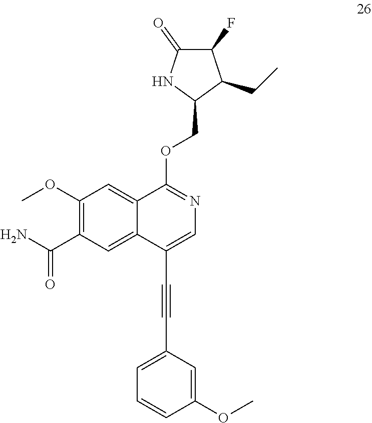 [SMILES: CC[C@@H]1[C@H](F)C(=O)N[C@@H]1COc1ncc(C#Cc2cccc(OC)c2)c2cc(C(N)=O)c(OC)cc12]

This compound was prepared by essential the same method as of example 12.

¹H NMR (400 MHz, DMSO-d₆, ppm) δ 8.91 (s, 1H), 8.40 (s, 1H), 8.24 (s, 1H), 7.95 (brs, 1H), 7.81 (br, 2H), 7.39 (t, 1H), 7.22 (d, 1H), 7.19 (d, 1H), 7.05 (dd, 1H), 4.91 (dd, Hz, 1H), 4.61-4.57 (m, 1H), 4.31 (dd, 1H), 4.12 (brs, 1H), 4.00 (s, 3H), 3.82 (s, 3H), 2.70-2.56 (m, 1H), 1.64-1.56 (m, 2H), 1.03 (t, 3H). MS m/z (ESI): 492.2 [M+H]⁺.

### Example 27: 1-(((2S,3S,4S)-3-ethyl-4-fluoro-5-oxopyrrolidin-2-yl)methoxy)-7-methoxy-4-(phenylethynyl)isoquinoline-6-carboxamide 27

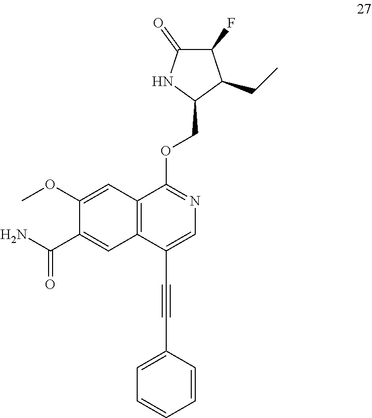 [SMILES: CC[C@@H]1[C@H](F)C(=O)N[C@@H]1COc1ncc(C#Cc2ccccc2)c2cc(C(N)=O)c(OC)cc12]

This compound was prepared by essential the same method as of example 12 to afford the title compound 27 (30 mg, 65.01 umol, 41.18% yield).

¹H NMR (400 MHz, DMSO-d6, ppm) δ 8.89 (s, 1H), 8.41 (s, 1H), 8.24 (s, 1H), 7.94 (s, 1H), 7.81 (s, 1H), 7.78 (brs, 1H), 7.67-7.63 (m, 2H), 7.51-7.47 (m, 3H), 4.91 (dd, 1H), 4.59 (dd, 1H), 4.31 (dd, 1H), 4.12 (br, 1H), 4.00 (s, 3H), 2.69-2.58 (m, 1H), 1.64-1.57 (m, 2H), 1.03 (t, 3H). MS m/z (ESI): 462.1 [M+H]⁺.

### Example 28: 1-(((2S,3S,4S)-3-ethyl-4-fluoro-5-oxopyrrolidin-2-yl)methoxy)-7-methoxy-4-(3-methoxy-3-methylbut-1-yn-1-yl)isoquinoline-6-carboxamide 28

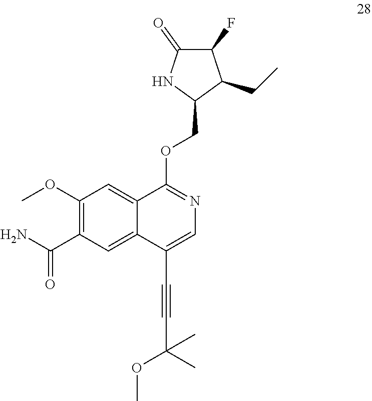 [SMILES: CC[C@@H]1[C@H](F)C(=O)N[C@@H]1COc1ncc(C#CC(C)(C)OC)c2cc(C(N)=O)c(OC)cc12]

This compound was prepared by essential the same method as of example 12 to afford the title compound 28 (15.8 mg, yield: 41.2%).

¹H NMR (400 MHz, DMSO-d₆, ppm) δ 8.88 (brs, 1H), 8.30 (s, 1H), 8.11 (s, 1H), 7.91 (brs, 1H), 7.78 (s, 1H), 7.74 (brs, 1H), 4.91 (dd, 1H), 4.56 (dd, 1H), 4.28 (dd, 1H), 4.10 (br, 1H), 3.99 (s, 3H), 3.38 (s, 3H), 2.69-2.55 (m, 1H), 1.62-1.57 (m, 2H), 1.57 (s, 6H), 1.02 (t, 3H). MS m/z (ESI): 456.1 [M−H].

### Example 29: 1-(((2S,3S,4S)-3-ethyl-4-fluoro-5-oxopyrrolidin-2-yl)methoxy)-4-(3-hydroxyl-3-methylbut-1-yn-1-yl)-7-isopropoxyisoquinoline-6-carboxamide 29

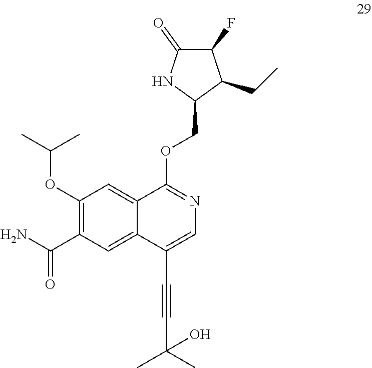 [SMILES: CC[C@@H]1[C@H](F)C(=O)N[C@@H]1COc1ncc(C#CC(C)(C)O)c2cc(C(N)=O)c(OC(C)C)cc12]

This compound was prepared by the same method as of example 25 to afford the product. ¹H NMR (400 MHz, CDCl₃, ppm): δ 8.8 (s, 1H), 8.7 (s, 1H), 8.1 (brs, 1H), 7.9 (s, 1H), 6.4 (brs, 1H), 5.1-4.8 (m, 2H), 4.7-4.5 (m, 1H), 4.5 (s, 2H), 4.2-4.1 (m, 1H), 2.8-2.5 (m, 1H), 2.3-2.1 (m, 1H), 1.8 (s, 3H), 1.7 (s, 3H), 1.8-1.5 (m, 2H), 1.5 (s, 3H), 1.4 (s, 3H), 1.1-0.9 (m, 3H). MS m/z (ESI): 472 [M+1].

### Example 30: 1-(((2S,3S,4S)-3-ethyl-4-fluoro-5-oxopyrrolidin-2-yl)methoxy)-7-methoxy-4-((2-methoxyphenyl)ethynyl)isoquinoline-6-carboxamide 30

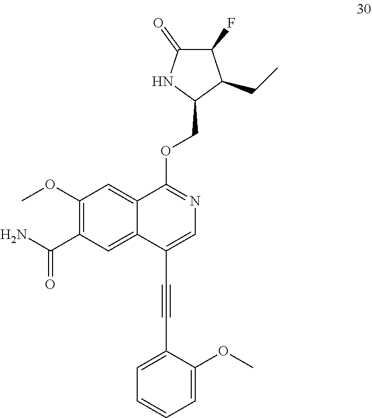 [SMILES: CC[C@@H]1[C@H](F)C(=O)N[C@@H]1COc1ncc(C#Cc2ccccc2OC)c2cc(C(N)=O)c(OC)cc12]

This compound was prepared by essential the same method as of example 12.

¹H NMR (400 MHz, DMSO-d₆, ppm) δ 8.91 (s, 1H), 8.58 (d, 1H), 7.95 (s, 1H), 7.81 (d, 1H), 7.80 (s, 1H), 7.53 (dd, 1H), 7.43 (td, 1H), 7.14 (d, 1H), 7.02 (td, 1H), 4.91 (dd, 1H), 4.59 (dd, 1H), 4.30 (dd, 1H), 4.12 (brs, 1H), 3.83 (s, 3H), 3.80 (s, 3H), 2.70-2.56 (m, 1H), 1.66-1.56 (m, 2H), 1.03 (t, 3H). MS m/z (ESI): 492.1 [M+H]⁺.

### Example 31: 5-(((2S,3S,4S)-3-ethyl-4-fluoro-5-oxopyrrolidin-2-yl)methoxy)-8-(3-hydroxyl-3-methylbut-1-yn-1-yl)-3-methoxy-2-naphthamide 31

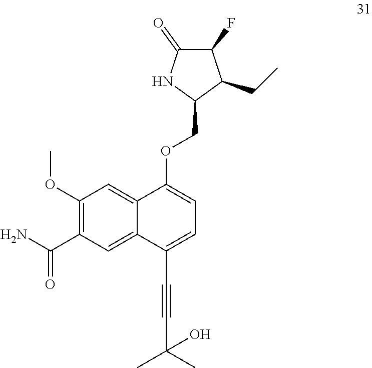 [SMILES: CC[C@@H]1[C@H](F)C(=O)N[C@@H]1COc1ccc(C#CC(C)(C)O)c2cc(C(N)=O)c(OC)cc12]

This compound was prepared by the same method as of example 15 to afford the product. ¹H NMR (400 MHz, CDCl₃, ppm): δ 9.0 (s, 1H), 8.0 (s, 1H), 7.7 (s, 1H), 7.4 (brs, 1H), 6.8-6.6 (m, 1H), 6.1 (brs, 1H), 5.1-4.9 (m, 1H), 4.3-4.1 (m, 3H), 3.9 (s, 3H), 2.8-2.6 (m, 1H), 1.8-1.6 (m, 8H), 1.2-1.0 (m, 3H). MS m/z (ESI): 443 [M+1].

### Example 32: 4-(3,3-dimethylbut-1-yn-1-yl)-1-(((2S,3S,4S)-3-ethyl-4-fluoro-5-oxopyrrolidin-2-yl)methoxy)-7-methoxyisoquinoline-6-carboxamide 32

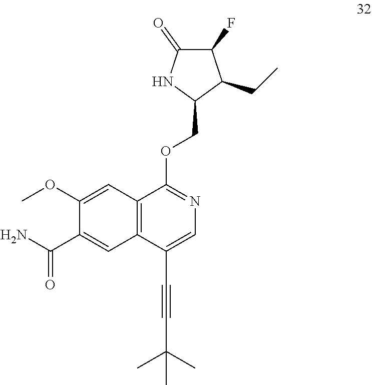 [SMILES: CC[C@@H]1[C@H](F)C(=O)N[C@@H]1COc1ncc(C#CC(C)(C)C)c2cc(C(N)=O)c(OC)cc12]

This compound was prepared by essential the same method as of example 12 to afford the title compound 32 (30 mg, 67.95 umol, 47.96% yield).

¹H NMR (400 MHz, DMSO-d₆, ppm) δ 8.87 (s, 1H), 8.30 (s, 1H), 8.01 (s, 1H), 7.90 (brs, 1H), 7.76 (s, 1H), 7.72 (brs, 1H), 4.91 (dd, 1H), 4.54 (dd, 1H), 4.26 (dd, 1H), 4.09 (br, 1H), 3.98 (s, 3H), 2.68-2.54 (m, 1H), 1.63-1.55 (m, 2H), 1.39 (s, 9H), 1.02 (t, 3H). MS m/z (ESI): 442.6 [M+H]⁺.

### Example 33: 8-(3-(dimethylamino)-3-methylbut-1-yn-1-yl)-5-(((2S,3S,4S)-3-ethyl-4-fluoro-5-oxopyrrolidin-2-yl)methoxy)-3-methoxy-2-naphthamide 33

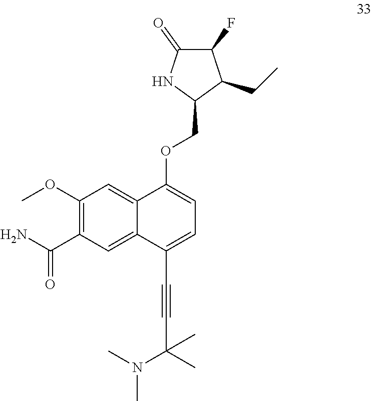 [SMILES: CC[C@@H]1[C@H](F)C(=O)N[C@@H]1COc1ccc(C#CC(C)(C)N(C)C)c2cc(C(N)=O)c(OC)cc12]

This compound was prepared by the same method as of example 15 to afford the product. ¹H NMR (400 MHz, CDCl₃, ppm): δ 9.1 (s, 1H), 7.8 (brs, 2H), 7.7 (s, 1H), 7.5-7.3 (m, 1H), 6.8-6.7 (m, 1H), 6.1 (brs, 1H), 5.1-4.9 (m, 1H), 4.3-4.1 (m, 3H), 4.0 (s, 3H), 2.8-2.6 (m, 1H), 2.5 (s, 6H), 1.9-1.6 (m, 2H), 1.6 (s, 6H), 1.2-1.0 (m, 3H). MS m/z (ESI): 470 [M+1].

### Example 34: 4-(3-(dimethylamino)-3-methylbut-1-yn-1-yl)-1-(((2S,3S,4S)-3-ethyl-4-fluoro-5-oxopyrrolidin-2-yl)methoxy)-7-methoxyisoquinoline-6-carboxamide 34

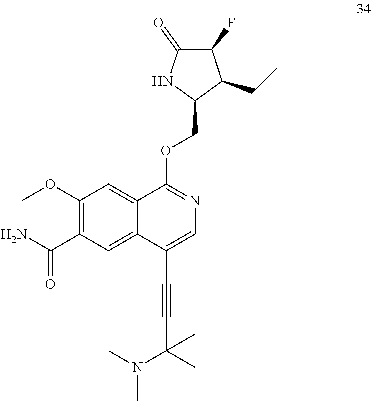 [SMILES: CC[C@@H]1[C@H](F)C(=O)N[C@@H]1COc1ncc(C#CC(C)(C)N(C)C)c2cc(C(N)=O)c(OC)cc12]

This compound was prepared by the same method as of example 1 to afford the product. ¹H NMR (400 MHz, CDCl₃, ppm): δ 8.9 (s, 1H), 8.1 (s, 1H), 7.9 (s, 1H), 7.7 (brs, 1H), 7.6 (s, 1H), 6.3 (brs, 1H), 5.0-4.8 (m, 2H), 4.5-4.4 (m, 1H), 4.3-4.2 (m, 1H), 4.0 (s, 3H), 3.2-3.0 (m, 1H), 2.5 (s, 6H), 1.8-1.6 (m, 2H), 1.5 (s, 6H), 1.2-1.1 (m, 3H). MS m/z (ESI): 471 [M+1].

### Example 35: 4-(3-(dimethylamino)-3-methylbut-1-yn-1-yl)-1-(((2S,3S,4S)-3-ethyl-4-fluoro-5-oxopyrrolidin-2-yl)methoxy)-7-isopropoxyisoquinoline-6-carboxamide 35

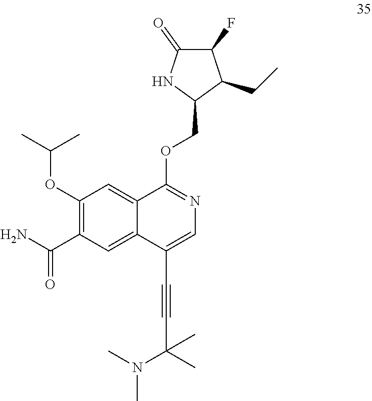 [SMILES: CC[C@@H]1[C@H](F)C(=O)N[C@@H]1COc1ncc(C#CC(C)(C)N(C)C)c2cc(C(N)=O)c(OC(C)C)cc12]

This compound was prepared by the same method as of example 25 to afford the product. ¹H NMR (400 MHz, CDCl₃, ppm): δ 9.1 (s, 1H), 8.2 (s, 1H), 8.1 (brs, 1H), 7.8 (s, 1H), 6.8 (s, 1H), 6.1 (brs, 1H), 5.1-4.7 (m, 3H), 4.6-4.4 (m, 1H), 4.2-4.1 (m, 1H), 2.8-2.5 (m, 1H), 2.4 (s, 6H), 1.9-1.7 (m, 2H), 1.6 (s, 6H), 1.6-1.4 (m, 6H), 1.2-1.0 (m, 3H). MS m/z (ESI): 499 [M+1].

### Biological Assays

The present disclosure will be further described with reference to the following test examples, but the examples should not be considered as limiting the scope of the disclosure.

### Test Example 1. IRAK4 Biochemical Assay

Experimental Procedure:

For the routine compound screening, an IRAK4 kinase assay was performed as follows using Promega ADP-Glo IRAK4 Kinase Enzyme System (Promega, Cat #2198-AD). Purified full length human IRAK4 protein (Signal Chem; Cat #I12-10G-125) was diluted to a concentration 6 nM in assay buffer (25 mM MOPS, pH 7.2, 12.5 mM β-glycerol-phosphate, 25 mM MgCl2, 5 mM EGTA, 2 mM EDTA, 0.0025% Brij-35, add 0.25 mM DTT to Kinase Assay Buffer prior to use) containing IRAK4 inhibitor at 2× the final concentration in 0.4% DMSO. The reaction was performed in PerkinElmer Proxiplate-384 plus white plate (Fisher Scientific, Cat #50-905-2761) and started by the addition of an equal volume of the assay buffer containing 4 μM native swine myelin basic protein (MBP) and 500 μM ATP to achieve a final concentration of 3 nM enzyme, 2 μM MBP, 250 μM ATP, 1× compound, and 0.2% DMSO. The kinase reaction was allowed to run for 60 min at room temperature (25-27° C.), then 5 μL of ADP-Glo was added to the reaction in each well followed by 40 minutes-incubation at RT. To detect converted ATP, 10 μL Detection Buffer was added into each well and Incubate at RT in dark for 30 minutes. Finally, luminescence was read using Tecan with 1000 ms of integration time. The luminescent signal positively correlates with ADP amount and kinase activity. Data was analyzed by GraphPad Prism and IC50 was calculated using function log(inhibitor) vs. response—Variable slope (four parameters). Percentage of inhibition was calculated as below:

% inhibition=(1−(Reading(Sample)−Reading(Negative Control))/(Reading(Positive Control)−Reading(Negative Control)))×100%

The 0% inhibition value comes from the Reading of Positive Control wells having 0 nM compound. The 100% inhibition value comes from the Reading of Negative Control wells having no IRAK4 kinase.

Data Analysis:

TABLE 1

| Example | IRAK4 Biochemical | IRAK4 Biochemical |
|---|---|---|
| No. | Assay (IC₅₀, nM) | Assay Max Inhibition (%) |
| 1 | 8.4 | 100.0 |
| 2 | 63.0 | 100.0 |
| 3 | 17.0 | 100.0 |
| 4 | 5.7 | 98.0 |
| 5 | 6.2 | 100.0 |
| 6 | 14.5 | 100.0 |
| 8 | 7.5 | 97.0 |
| 9 | 14.0 | 100.0 |
| 10 | 68.0 | 100.0 |
| 11 | 10.0 | 95.0 |
| 12 | 69.0 | 100.0 |
| 13 | 18.0 | 95.0 |
| 14 | 29.0 | 100.0 |
| 15 | 12.0 | 101.0 |
| 16 | 19.0 | 99.0 |
| 17 | 32.0 | 100.0 |
| 18 | 27.5 | 95.0 |
| 19 | 13.0 | 100.0 |
| 20 | 22.0 | 100.0 |
| 21 | 22.0 | 98.0 |
| 22 | 23.0 | 100.0 |
| 25 | 27.0 | 103.0 |
| 27 | 24.0 | 100.0 |
| 28 | 37.0 | 100.0 |
| 29 | 26.0 | 100.0 |
| 31 | 32.0 | 95.0 |
| 32 | 45.0 | 99.0 |
| 33 | 13.0 | 99.0 |
| 34 | 8.9 | 92.0 |
| 35 | 39.0 | 93.0 |

Conclusion: The compounds of the present disclosure have a significant inhibition effect on the enzymatic activity of IRAK4.

### Test Example 2. IRAK4 Human PBMC TNFα, Cell Based Assay

Experimental Procedure:

In this assay, serially diluted compounds were incubated with 1×10⁴ human peripheral blood mononuclear cells (PBMCs) from STEMCELL (Cat #70025.1) per well of 384-well plate (Fisher Scientific; 50-905-2761) and cultured with TexMACS medium (Miltenyi, Cat #130-097-196), stimulated by R848 (0.5 μM) or IL-1β (10 ng/ml). Plates were covered with lids and incubated for 4 h (R848, Fisher Scientific, Cat #NC9801605) or 24 h (IL-1β, Fisher Scientific, Cat #ENRIL1BI) at 37° C. in a humidified tissue-culture incubator. After a brief centrifugation at 1000 rpm for 5 minutes, 5 μl of culture medium was used for the measurement of the TNFα amount using TNFα (human) AlphaLISA Detection Kit (PerkinElmer, Cat #AL208C) according to manufacturer's protocol. A biotinylated anti-TNFα antibody binds to the Streptavidin-coated Donor beads while another anti-TNFα antibody is conjugated to AlphaLISA Acceptor beads. In the presence of the TNFα, the beads come into close proximity. The excitation of the Donor beads causes the release of singlet oxygen molecules that triggers a cascade of energy transfer in the Acceptor beads, resulting in a sharp peak of light emission at 615 nm. AlphaLISA assay plates were read using PHERAstar. Percentage of inhibition was calculated as below:

% inhibition=(1−(Reading(Sample)−Reading(Negative Control))/(Reading(Positive Control)−Reading(Negative Control)))×100%

The 0% inhibition value comes from the Reading of Positive Control wells having 0 nM compound. The 100% inhibition value comes from the Reading of Negative Control wells having no R848 or IL-1β.

TABLE 2

| Example | IRAK4 PBMC AlphaLISA | IRAK4 PBMC AlphaLISA |
|---|---|---|
| No. | R848 (IC₅₀, nM) | IL-1β (IC₅₀, nM) |
| 1 | 16.0 | 18.1 |
| 2 | 16.0 | 25.0 |
| 3 | 2.7 | 4.7 |
| 5 | 0.7 | 2.2 |
| 6 | 1.0 | 1.3 |
| 7 | 150.0 | 89.0 |
| 8 | 175.0 | 128.5 |
| 9 | 140.0 | 110.0 |
| 11 | 0.7 | 1.0 |
| 12 | 1.4 | 0.8 |
| 13 | 1.6 | 1.3 |
| 14 | 1.6 | 1.3 |
| 15 | 1.7 | 1.3 |
| 16 | 1.7 | 8.7 |
| 17 | 2.4 | 1.5 |
| 18 | 2.5 | 1.7 |
| 19 | 3.0 | 1.3 |
| 20 | 3.0 | 1.7 |
| 21 | 3.1 | 4.6 |
| 22 | 3.4 | 0.8 |
| 25 | 5.0 | 3.9 |
| 27 | 5.3 | 1.8 |
| 28 | 5.5 | 9.2 |
| 29 | 6.7 | 3.5 |
| 31 | 8.3 | 5.6 |
| 32 | 11.0 | 17.0 |
| 33 | 25.0 | 41.0 |
| 34 | 32.0 | 46.0 |
| 35 | 36.0 | 110.0 |

Data Analysis:

Conclusion: The compounds of the present disclosure have a significant inhibition effect on the cellular activity of IRAK4.

### Test Example 3. In Vitro Study for Inhibiting Cytochrome P450 (CYP) 2D6 Using Human Liver Microsomes

### 1. Chemical Reagents and Buffers

Potassium phosphate buffer (200 ferateas purchase from Sangon Biotech. Dilute 20×PBS to 100 mM PBS buffer. The stock solutions of NADPH was 5 mM and the final concentration in the incubation system was 1 mM. The stock solution of MgCl2 were 7.5 mM and t the final concentration in the incubation system was 3 mM.

1.1 Preparation of probe substrate solution: The stock solutions of probe substrates of dextromethorphan (Sigma Cat No. D9684) was prepared by using DMSO and the final concentration in incubation system was 4 μM.

1.2 Preparation of selective inhibitors solution: The 30 mM stock solutions of inhibitor of CYP isoforms of Quinidine (Sigma, Q0750) was prepared by using DMSO and the final concentration in incubation system were 30, 10, 3, 1, 0.3, 0.03, 0.003 μM.

1.3 Preparation of test compound 5 and 6 solution: The 30 mM test compound 5 and 6 solutions were prepared by using DMSO and the final concentration in incubation system were 30, 10, 3, 1, 0.3, 0.03, 0.003 μM.

1.4 Human liver microsomes (HLM) were purchased by Corning Co, LTD. Diluted the HLM into 0.25 mg/ml and the final concentration was 0.1 mg/ml.

### 2. Procedure:

Added 20 μL working solutions of test compound 5 or 6 or selective inhibitors into a 96-well plate. Then added 40 μL of the HLM working solution and 20 μL of the substrate working solution. Pre-warmed the plate for about 5 min at 37° C. water bath. After that, added 20 μL NADPH/MgCl2 solution. Mixed and incubated the plate for 30 minutes at 37° C. Then terminated the reaction by adding 250 μL cold acetonitile (containing 100 ng/mL internal standard, Camptothecin, National Institutes for Food and Drug Control). Centrifuged the plate at 4000 rpm for 20 minutes to precipitate protein. Dilute 100 μL supernatant with 80 μL water, and shake for 10 min. Samples are now ready for LC/MS/MS analysis.

### 3. Results

The IC50 value of test compounds inhibit on CYP2D6 was calculated by Graphpad Prism and shown in Table.

| Compounds | IC₅₀(μM) |
|---|---|
| 5 | >30 |
| 6 | 13.7 |

### 4. Conclusion

The compound has a weak inhibitory effect on the CYP2D6.

1. A compound of formula (I), or a tautomer, mesomer, racemate, enantiomer, diastereomer, or mixture thereof, or a pharmaceutically acceptable salt thereof: 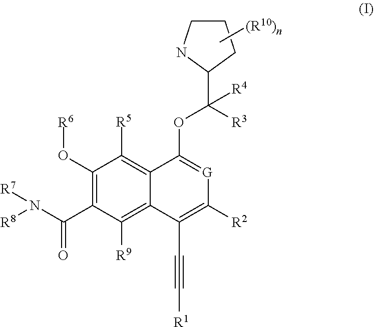
wherein:
G is N or CR^{g};
R¹ is selected from C₄₋₆ alkyl, alkyl substituted by R, —CONR^{m}Rⁿ, —COOR^{p}, aryl and heteroaryl, wherein the aryl or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ᵣ—NR^{a}R^{b}, —C(═O)R^{c}, —OC(═O)R^{c}, —OC(═O)OR^{a}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl;
R is selected from halogen, alkyl, haloalkyl, alkoxy, haloalkoxy, hydroxyl, hydroxyalkyl, cyano, amino, nitro, —(CH₂)ᵣ—NR^{a}R^{b}, —C(═O)R^{c}, —C(═O)OR^{a}, —OC(═O)R^{c}, —C(═O)NR^{a}R^{b}, —NR^{d}C(═O)R^{c}, —NR^{d}C(═O)OR^{a}, —SO₂R^{a}, —SO₂NR^{a}R^{b}, —NR^{d}SO₂R^{a}, cycloalkyl, heterocyclyl, aryl and heteroaryl;
R², R⁵, R⁹ and R^{g} are identical or different and are independently selected from hydrogen, halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b}, nitro, cycloalkyl, heterocyclyl, aryl and heteroaryl; wherein the alkyl, cycloalkyl, heterocyclyl, aryl or heteroaryl is optionally substituted with one or more groups independently selected from halogen, alkyl, haloalkyl, alkoxy, haloalkoxy, hydroxyl, hydroxyalkyl, cyano, amino, nitro, —(CH₂)ₛ—NR^{e}R^{f}, cycloalkyl, heterocyclyl, aryl and heteroaryl;
R³ and R⁴ are identical or different and are independently selected from hydrogen, halogen, alkyl, haloalkyl, alkoxy, alkylthio, haloalkoxy, haloalkylthio, hydroxyl, hydroxyalkyl, cyano, amino and —(CH₂)ᵣ—NR^{a}R^{b};
R⁶ is selected from alkyl, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more groups independently selected from halogen, alkyl, haloalkyl, alkoxy, alkylthio, haloalkoxy, hydroxyl, hydroxyalkyl, cyano, amino, nitro, —(CH₂)ₛ—NR^{e}R^{f} and cycloalkyl;
R¹⁰ at each occurrence is independently selected from hydrogen, halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b}, nitro, oxo, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more groups independently selected from halogen, alkyl, haloalkyl, alkoxy, alkylthio, haloalkoxy, haloalkylthio, hydroxyl, hydroxyalkyl, cyano, amino, nitro, —(CH₂)ₛ—NR^{e}R^{f} and cycloalkyl;
or two R¹⁰ together with the attached atoms form a cycloalkyl or heterocyclyl; wherein the cycloalkyl and heterocyclyl are optionally substituted with one or more groups independently selected from halogen, alkyl, haloalkyl, alkoxy, alkylthio, haloalkoxy, haloalkylthio, hydroxyl, hydroxyalkyl, cyano, amino, nitro and —(CH₂)ₛ—NR^{e}R^{f};
R⁷, R⁸, R^{a}, R^{b}, R^{c}, R^{f}, R^{p}, R^{m} and Rⁿ are identical or different and at each occurrence are independently selected from hydrogen, alkyl, haloalkyl, hydroxyalkyl, —C(═O)OR^{q}, cycloalkyl, heterocyclyl, aryl and heteroaryl;
or R⁷ and R⁸, R^{a} and R^{b}, R^{e} and R^{f}, R^{m} and Rⁿ together with the nitrogen to which they are attached form a heterocyclyl; wherein the heterocyclyl is optionally substituted with one or more groups independently selected from halogen, alkyl, haloalkyl, alkoxy, haloalkoxy, hydroxyl, hydroxyalkyl, oxo, cyano, amino, nitro, cycloalkyl, heterocyclyl, aryl and heteroaryl;
R^{c} at each occurrence is independently selected from alkyl, cycloalkyl, heterocyclyl, aryl and heteroaryl, wherein the alkyl, cycloalkyl, heterocyclyl, aryl or heteroaryl is optionally substituted with one or more groups independently selected from halogen, alkyl, haloalkyl, alkoxy, haloalkoxy, hydroxyl, hydroxyalkyl, cyano, amino and nitro;
R^{d} at each occurrence are independently selected from hydrogen, alkyl, haloalkyl, hydroxyalkyl and cycloalkyl, wherein the cycloalkyl is optionally substituted with one or more groups independently selected from halogen, alkyl, haloalkyl, alkoxy, haloalkoxy, hydroxyl, hydroxyalkyl, cyano, amino, and nitro;
R^{q} is selected from hydrogen, alkyl and haloalkyl;
n is 0, 1, 2, 3 or 4;
r is 0, 1, 2 or 3; and
s is 0, 1, 2 or 3.

2. The compound of formula (I), or a tautomer, mesomer, racemate, enantiomer, diastereomer, or mixture thereof, or a pharmaceutically acceptable salt thereof according to claim 1, wherein R¹ is aryl or heteroaryl, wherein the aryl or heteroaryl is optionally substituted with one or more groups independently selected from halogen, alkyl, haloalkyl, alkoxy, haloalkoxy, hydroxyl, hydroxyalkyl, cyano, amino and nitro.

3. The compound of formula (I), or a tautomer, mesomer, racemate, enantiomer, diastereomer, or mixture thereof, or a pharmaceutically acceptable salt thereof according to claim 1, wherein R¹ is selected from alkyl substituted by R, —CONR^{m}Rⁿ and —COOR^{p};
R is selected from halogen, alkyl, haloalkyl, alkoxy, haloalkoxy, hydroxyl, hydroxyalkyl, cyano, amino, nitro, —(CH₂)ᵣ—NR^{a}R^{b}, cycloalkyl, heterocyclyl, aryl and heteroaryl;
wherein R^{a}, R^{b}, R^{p}, R^{m}, Rⁿ and r are defined in claim 1.

4. The compound of formula (I), or a tautomer, mesomer, racemate, enantiomer, diastereomer, or mixture thereof, or a pharmaceutically acceptable salt thereof according to claim 1, being a compound of formula (II), or a tautomer, mesomer, racemate, enantiomer, diastereomer, or mixture thereof, or a pharmaceutically acceptable salt thereof: 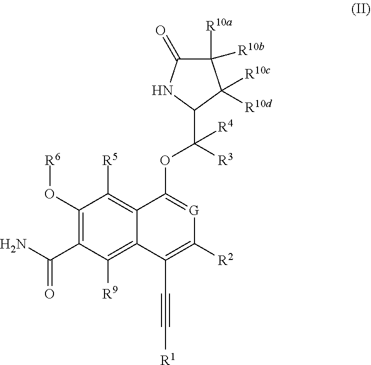
wherein:
R^{10a}, R^{10b}, R^{10c} and R^{10d} are identical or different and at each occurrence are independently selected from hydrogen, halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b} and cycloalkyl;
G, R¹-R⁶ and R⁹ are each as defined in claim 1.

5. The compound of formula (I), or a tautomer, mesomer, racemate, enantiomer, diastereomer, or mixture thereof, or a pharmaceutically acceptable salt thereof according to claim 1, being a compound of formula (III), or a tautomer, mesomer, racemate, enantiomer, diastereomer, or mixture thereof, or a pharmaceutically acceptable salt thereof: 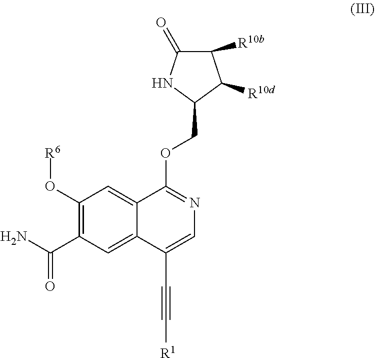
wherein:
R^{10b} and R^{10d} are identical or different and at each occurrence are independently selected from halogen, alkyl, haloalkyl, alkoxy, alkylthio, haloalkoxy, haloalkylthio, hydroxyl, hydroxyalkyl, cyano, amino, —(CH₂)ᵣ—NR^{a}R^{b} and cycloalkyl;
R¹, R⁶, R^{a}, R^{b} and r are each as defined in claim 1.

6. The compound of formula (I), or a tautomer, mesomer, racemate, enantiomer, diastereomer, or mixture thereof, or a pharmaceutically acceptable salt thereof according to claim 1, wherein R² is hydrogen, halogen or C₁₋₆ alkyl.

7. The compound of formula (I), or a tautomer, mesomer, racemate, enantiomer, diastereomer, or mixture thereof, or a pharmaceutically acceptable salt thereof according to claim 1, wherein R³ is hydrogen or C₁₋₆ alkyl.

8. The compound of formula (I), or a tautomer, mesomer, racemate, enantiomer, diastereomer, or mixture thereof, or a pharmaceutically acceptable salt thereof according to claim 1, wherein R⁴ is hydrogen or C₁₋₆ alkyl.

9. The compound of formula (I), or a tautomer, mesomer, racemate, enantiomer, diastereomer, or mixture thereof, or a pharmaceutically acceptable salt thereof according to claim 1, wherein R⁵ is hydrogen, halogen or C₁₋₆ alkyl.

10. The compound of formula (I), or a tautomer, mesomer, racemate, enantiomer, diastereomer, or mixture thereof, or a pharmaceutically acceptable salt thereof according to claim 1, wherein R⁹ is hydrogen, halogen or C₁₋₆ alkyl.

11. The compound of formula (I), or a tautomer, mesomer, racemate, enantiomer, diastereomer, or mixture thereof, or a pharmaceutically acceptable salt thereof according to claim 1, wherein each R⁶ is C₁₋₆ alkyl.

12. The compound of formula (I), or a tautomer, mesomer, racemate, enantiomer, diastereomer, or mixture thereof, or a pharmaceutically acceptable salt thereof according to claim 1, wherein R⁷ is hydrogen or C₁₋₆ alkyl.

13. The compound of formula (I), or a tautomer, mesomer, racemate, enantiomer, diastereomer, or mixture thereof, or a pharmaceutically acceptable salt thereof according to claim 1, wherein R⁸ is hydrogen or C₁₋₆ alkyl.

14. The compound of formula (I), or a tautomer, mesomer, racemate, enantiomer, diastereomer, or mixture thereof, or a pharmaceutically acceptable salt thereof according to claim 1, wherein R¹⁰ is selected from hydrogen, halogen, C₁₋₆ alkyl and oxo.

15. The compound of formula (I), or a tautomer, mesomer, racemate, enantiomer, diastereomer, or mixture thereof, or a pharmaceutically acceptable salt thereof according to claim 1, wherein the compound is selected from:

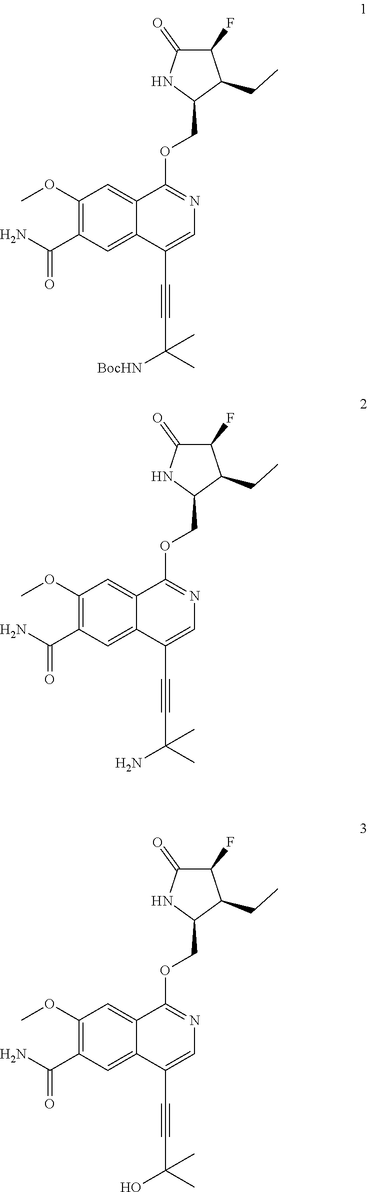

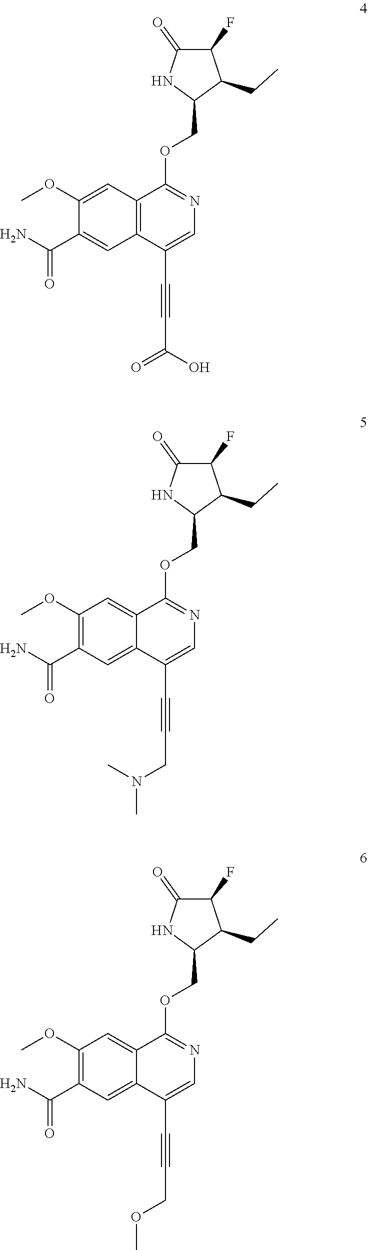

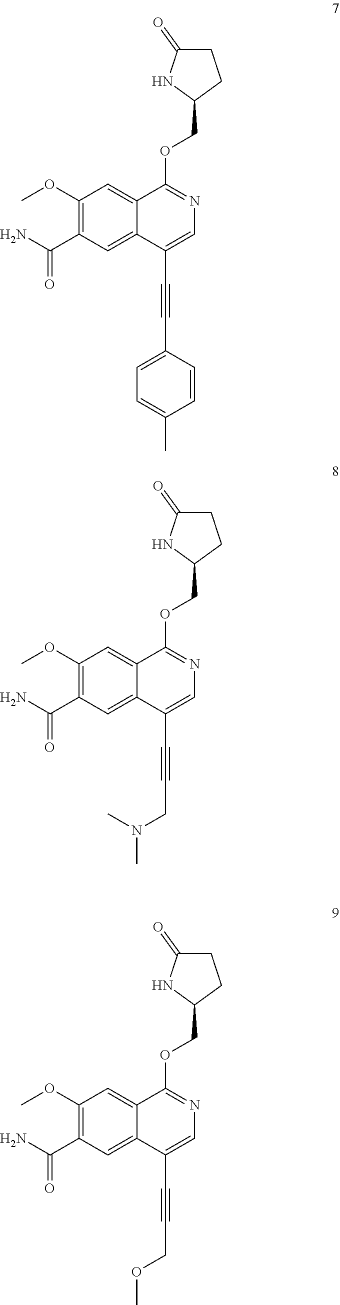

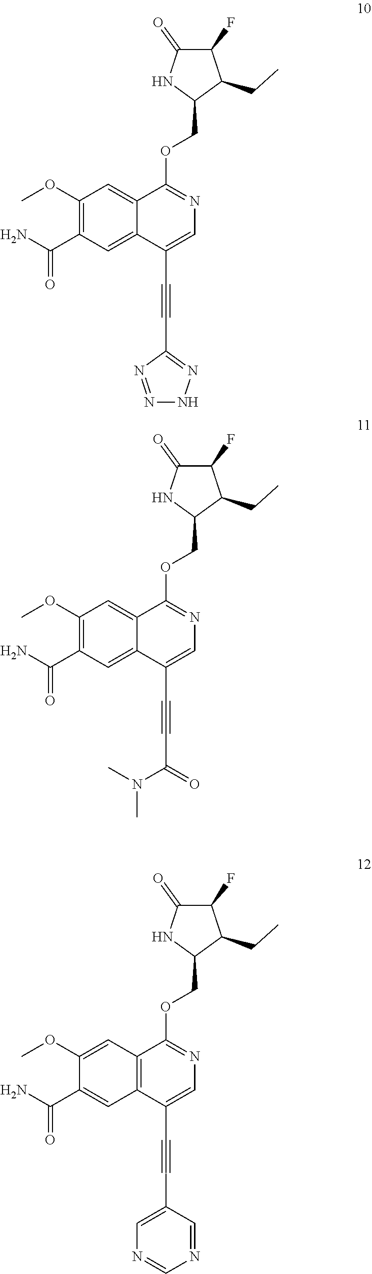

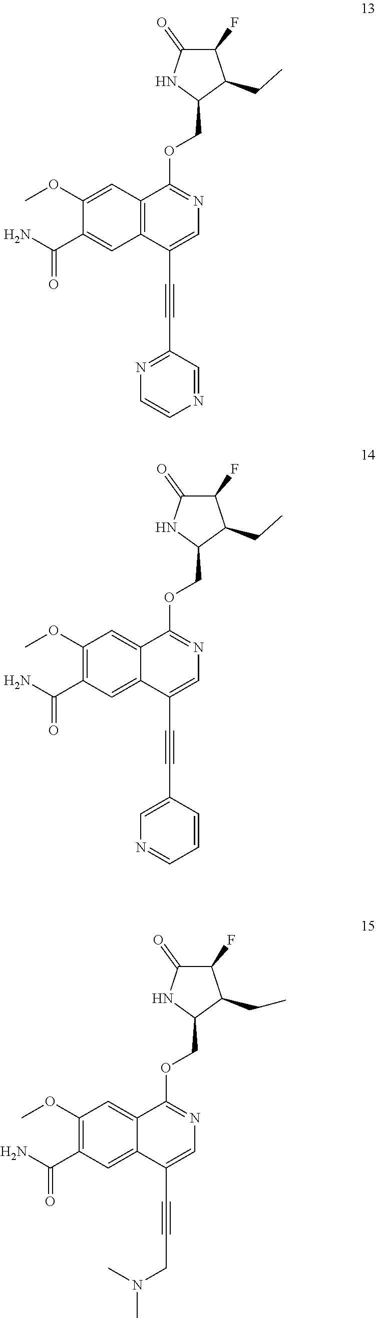

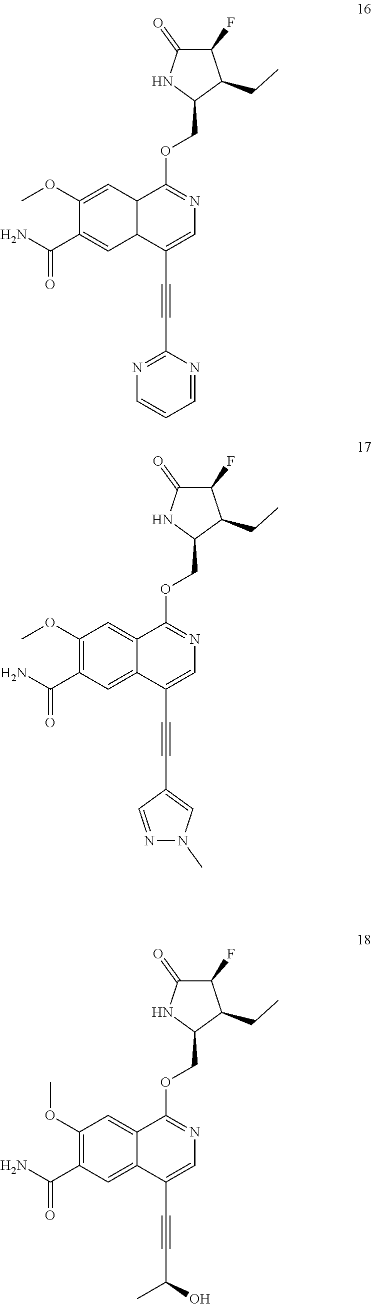

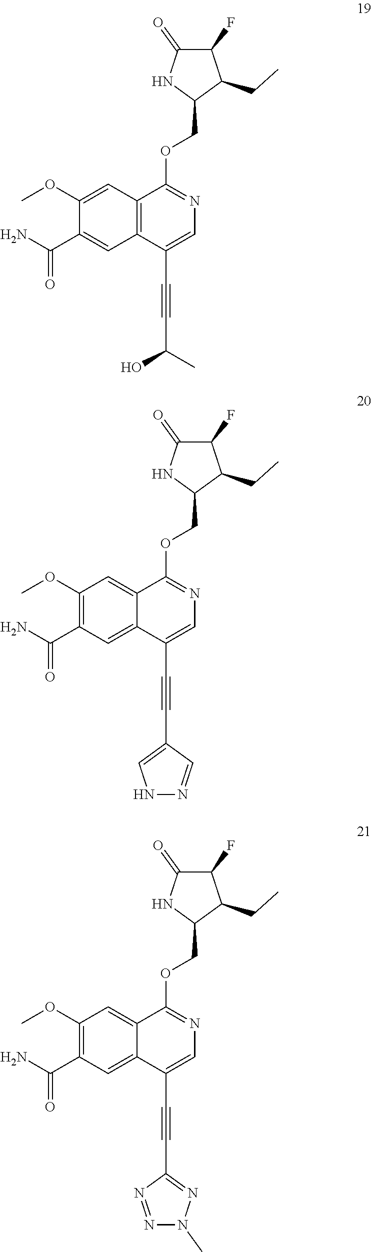

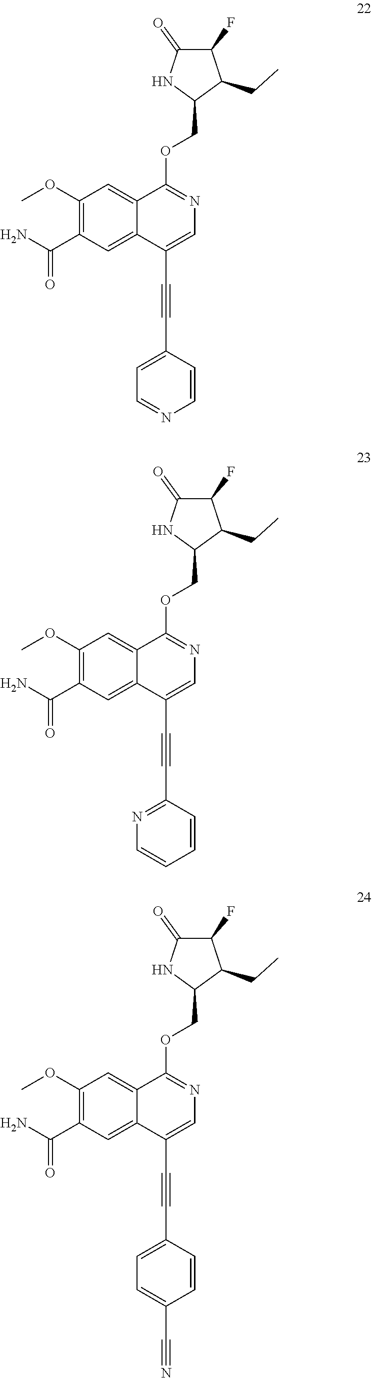

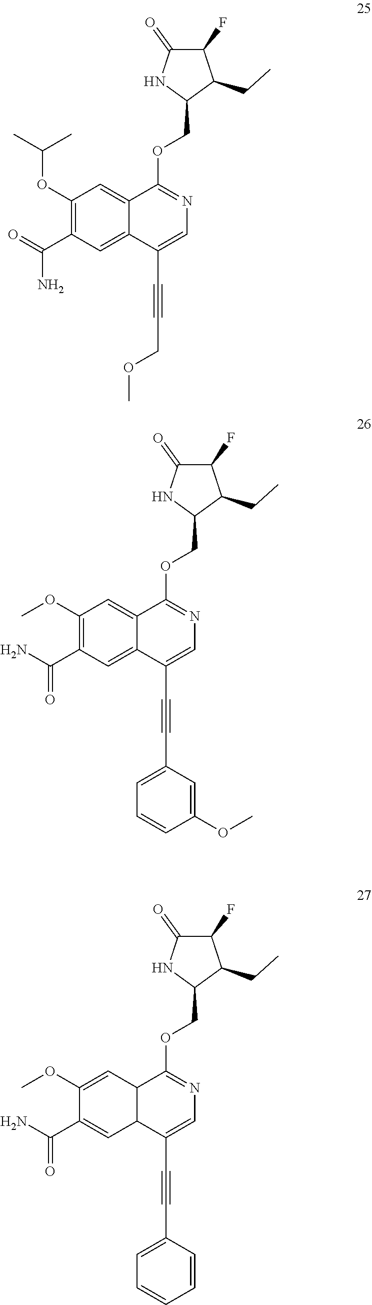

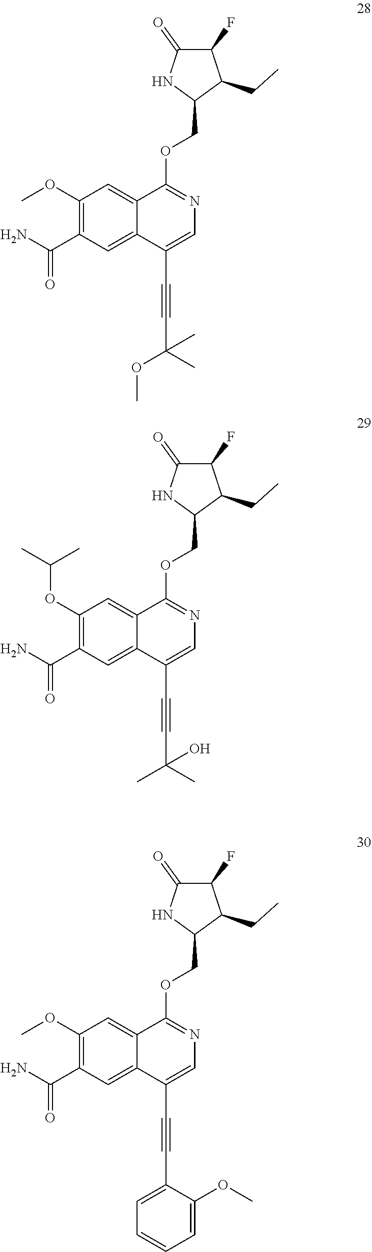

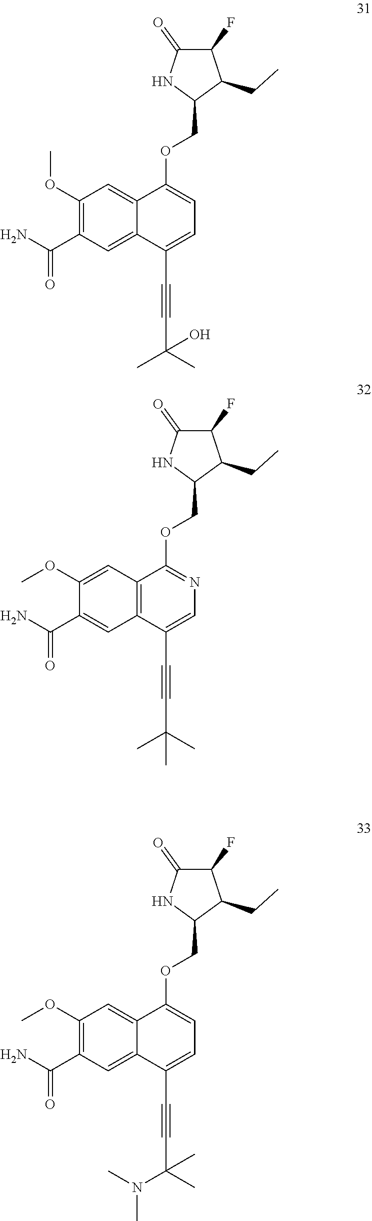

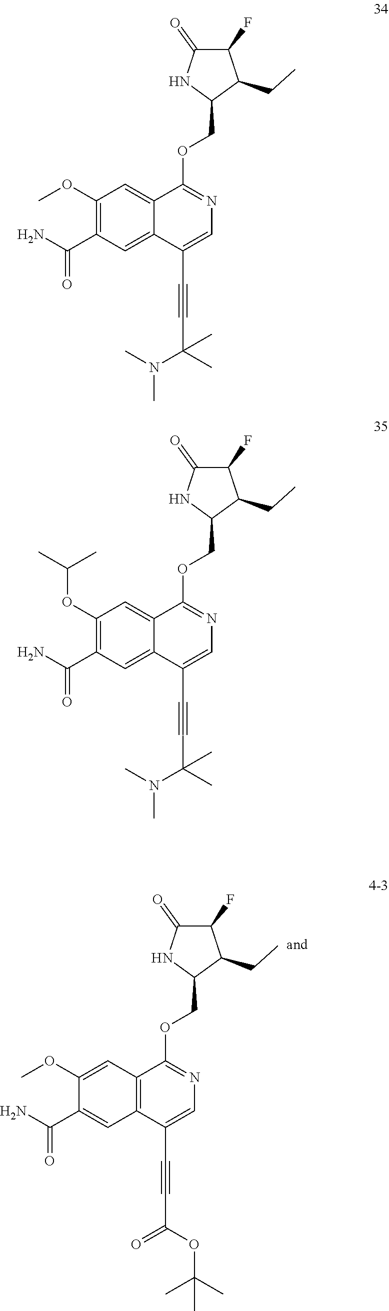

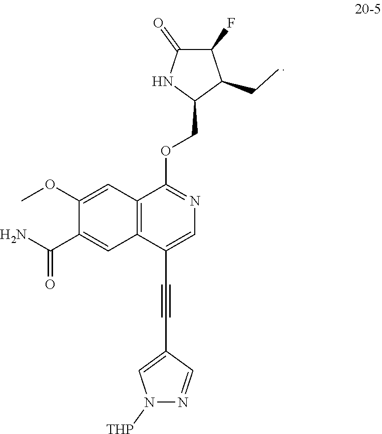

16. A compound of formula (IA1), or a tautomer, mesomer, racemate, enantiomer, diastereomer, or mixture thereof, or a pharmaceutically acceptable salt thereof: 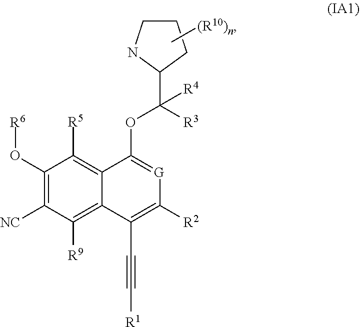
wherein:
G, R¹-R⁶, R⁹, R¹⁰ and n are each as defined in claim 1.

17. A compound of formula (IIA2), or a tautomer, mesomer, racemate, enantiomer, diastereomer, or mixture thereof, or a pharmaceutically acceptable salt thereof: 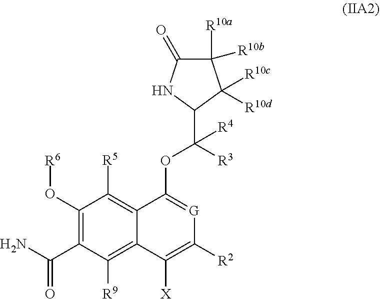
wherein:
X is Cl, Br, I, or OTf;
G is CR^{g};
R^{g}, R²-R⁶, R⁹, and R^{10a}-R^{10d} are each as defined in claim 4.

18. The compound of formula (IA1) or (IIA2), or a tautomer, mesomer, racemate, enantiomer, diastereomer, or mixture thereof, or a pharmaceutically acceptable salt thereof according to claim 16, wherein the compound is selected from:

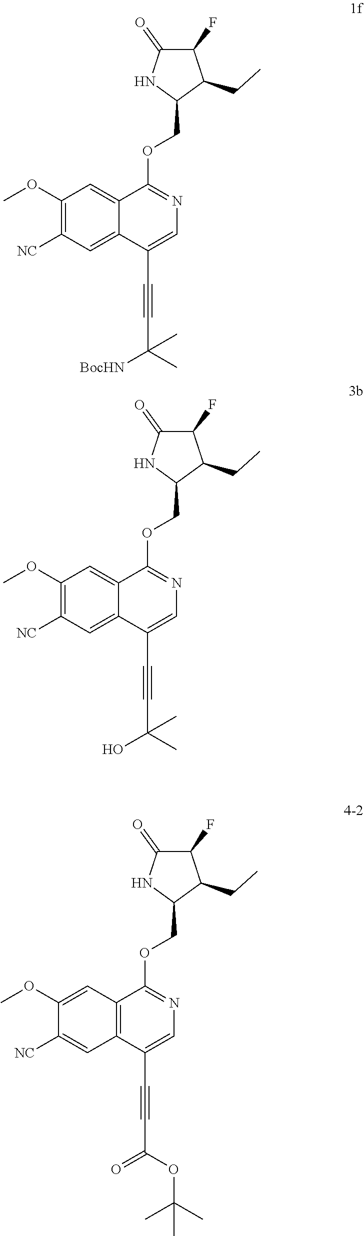

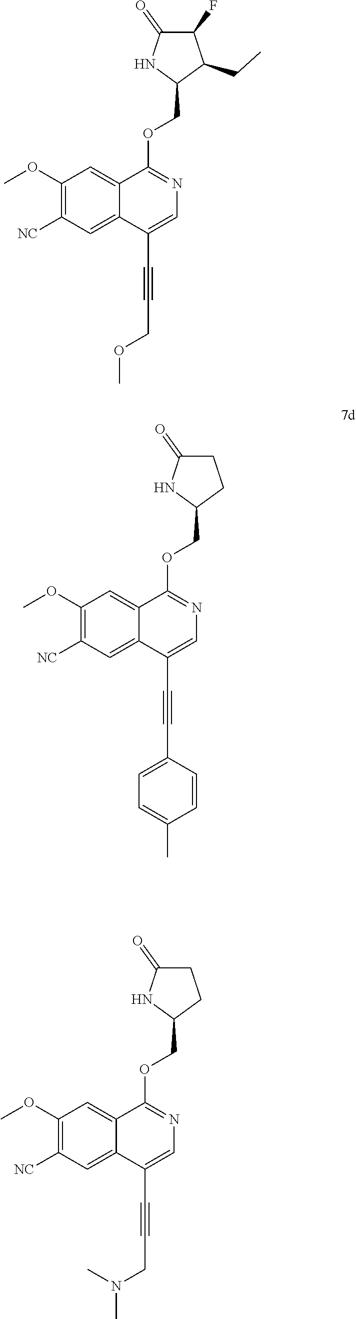

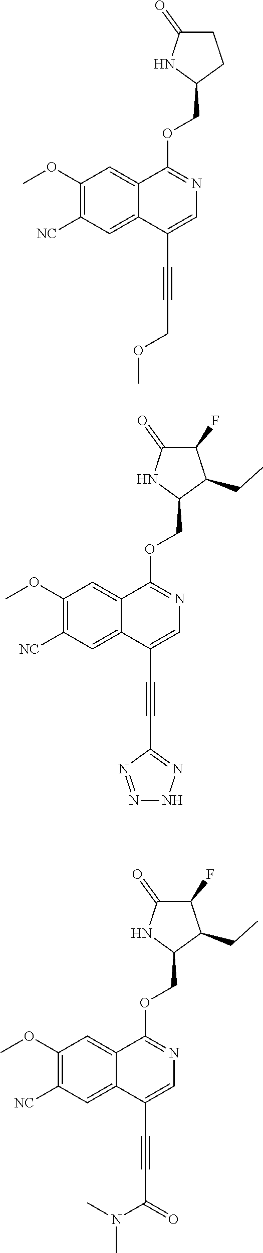

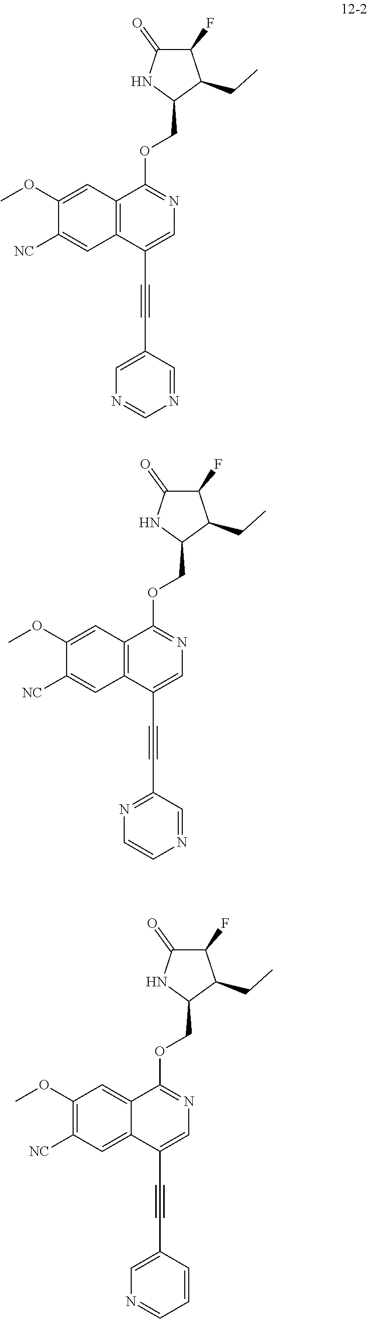

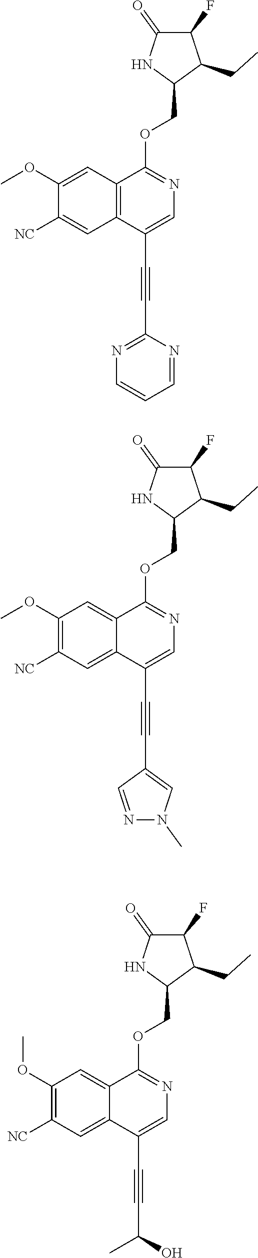

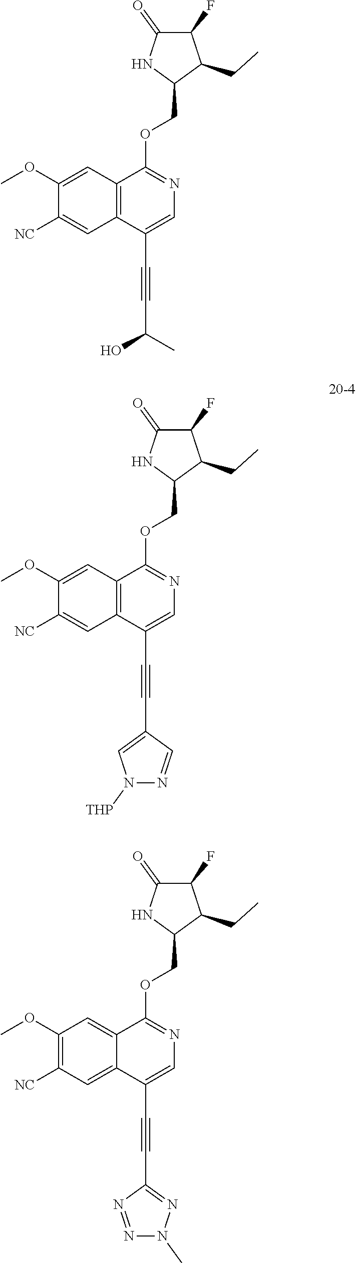

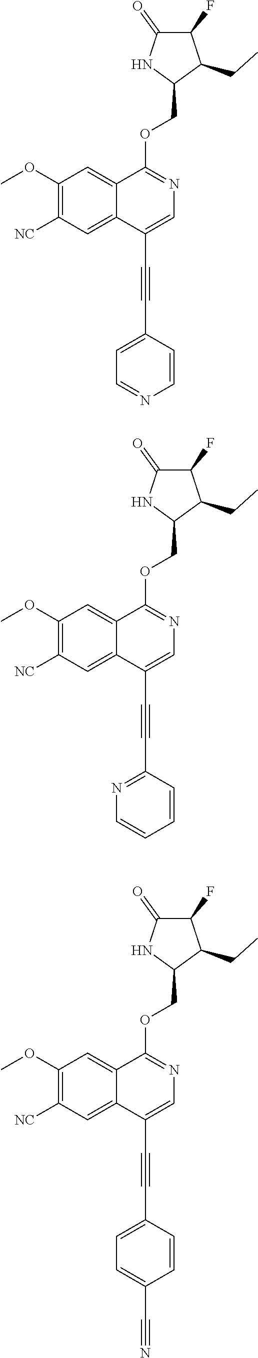

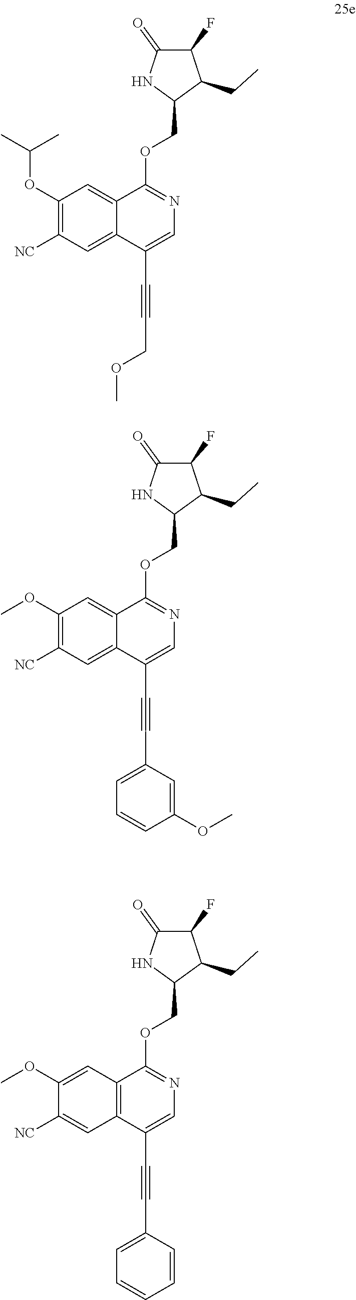

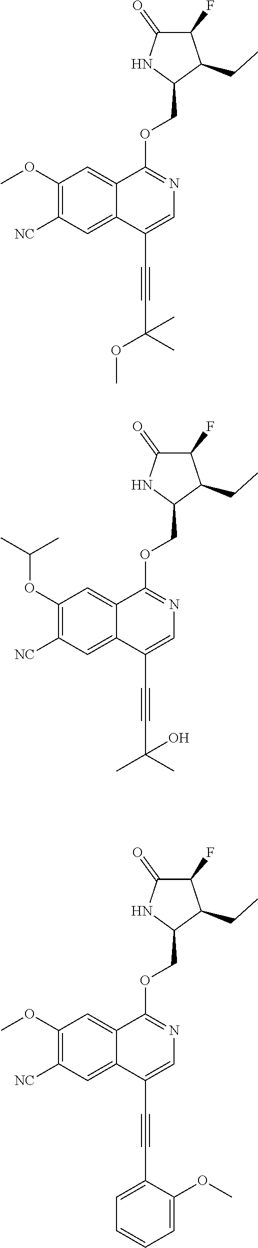

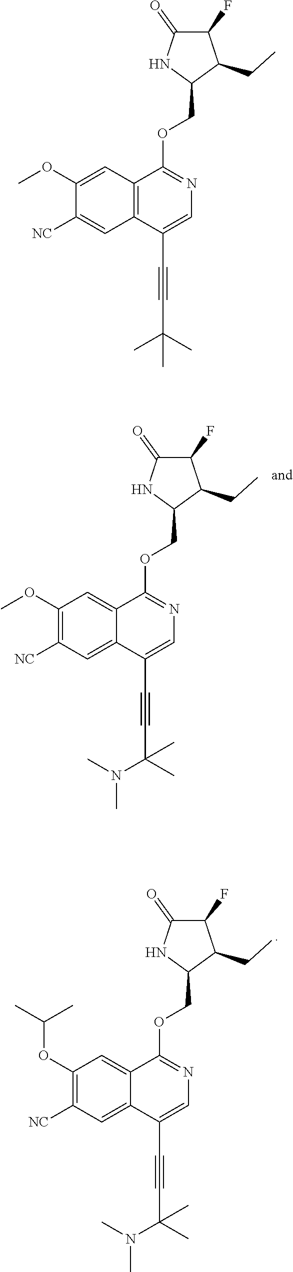

19. A process of preparing the compound of formula (I) according to claim 1, or a tautomer, mesomer, racemate, enantiomer, diastereomer, or mixture thereof, or a pharmaceutically acceptable salt thereof, comprising a step of: 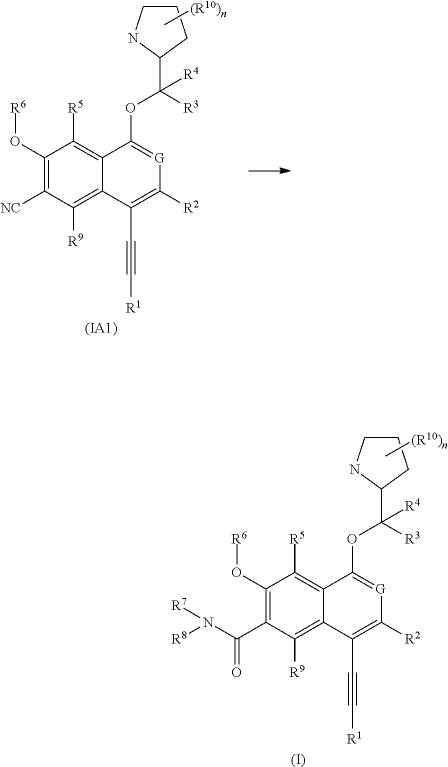
subjecting a compound of formula (IA1) to a hydration reaction to obtain the compound of formula (I), wherein:
R⁷ is hydrogen;
R⁸ is hydrogen; and
G, R¹ to R⁶, R⁹, R¹⁰ and n are each as defined in claim 1.

20. A process of preparing the compound of formula (I) according to claim 1, or a tautomer, mesomer, racemate, enantiomer, diastereomer, or mixture thereof, or a pharmaceutically acceptable salt thereof, comprising a step of: 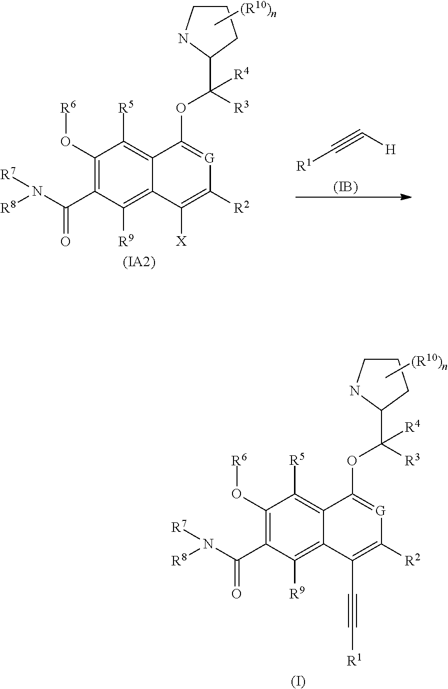
reacting a compound of formula (IA2) with a compound of formula (IB) to obtain the compound of formula (I), wherein:
X is halogen or OTf; and
G, R¹ to R¹⁰ and n are each as defined in claim 1.

21. A pharmaceutical composition, comprising a compound according to claim 1, or a tautomer, mesomer, racemate, enantiomer, diastereomer, or mixture thereof, or a pharmaceutically acceptable salt thereof, and a pharmaceutically acceptable carrier.

22. A method of preventing and/or treating an IRAK-mediated disorder, disease, or condition, comprising administering to a subject in need thereof a therapeutically effective amount of a compound according to claim 1, or a tautomer, mesomer, racemate, enantiomer, diastereomer, or mixture thereof, or a pharmaceutically acceptable salt thereof, or a pharmaceutical composition thereof; preferably the IRAK-mediated disorder, disease or condition is selected from a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

23. The method according to claim 22, wherein the cancer or proliferative disorder is selected a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, nonsmall-cell lung carcinoma, lymphomas, Hodgkins and Non-Hodgkins, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABCDLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulmemia (WM), splenic marginal zone lymphoma, multiple myeloma, plasmacytoma, and intravascular large B-cell lymphoma).

24. The method according to claim 23, wherein the MyD88 driven disorder is selected from ABC DLBCL, Waldenstrom's macroglobulmemia, Hodgkin's lymphoma, primary cutaneous T-cell lymphoma and chronic lymphocytic leukemia; and/or the IL-1 driven disorder is Smoldering of indolent multiple myeloma; and/or the neurodegenerative disease is selected from Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease; and/or the inflammatory disorder is selected from conditions of the eye such as ocular allergy, conjunctivitis, keratoconjunctivitis sicca, and vernal conjunctivitis; diseases affecting the nose including allergic rhinitis; and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Juvenile rheumatoid arthritis, Cryopyrin Associated Periodic Syndrome (CAPS), and osteoarthritis.
